(19) United States
(12) Patent Application Publication
(10) Pub. No.: US 2026/0120841 A1
(43) Pub. Date: Apr. 30, 2026

(54) NETWORK ROUTING SYSTEM

(71) Applicant: Scripventures Group, Ltd., Spring, TX (US)

(72) Inventors: Keith M. JACOBS, Plano, TX (US); Paul J. THOMAS, Spring, TX (US); Stephen G. THOMAS, Spring, TX (US); Gregory P. ANDRESS, Plano, TX (US)

(73) Assignee: Scripventures Group, Ltd., Spring, TX (US)

(21) Appl. No.: 19/429,880

(22) Filed: Dec. 22, 2025

Related U.S. Application Data
(63) Continuation-in-part of application No. 19/263,371, filed on Jul. 8, 2025, now Pat. No. 12,568,043.
Application No. 19/263,371 is a continuation of application No. 17/192,538, filed on Mar. 4, 2021, now abandoned.

(51) Int. Cl. G16H 20/10; G06Q 40/08

(52) U.S. Cl. CPC G16H 20/10; G06Q 40/08

(57) ABSTRACT

Various examples, systems, and methods are disclosed relating to network routing. The systems and methods can store, in a network user dataset, network plan information for a plurality of users, the network plan information for at least one user of the plurality of users including a first set of routing attributes. The systems and methods can associate, in the network user dataset, a second set of routing attributes with the first set of routing attributes for the at least one user. The systems and methods can receive, from the computer network originator system, a package including the second set of routing attributes associated with the at least one user. The systems and methods can replace, for the package, the second set of routing attributes with the first set of routing attributes. The systems and methods can send the package including the updated routing information to the computer network processing system.

### CROSS-REFERENCE TO RELATED PATENT APPLICATIONS

The present application is a continuation-in-part of U.S. patent application Ser. No. 19/263,371, filed Jul. 8, 2025, which is a continuation of U.S. patent application Ser. No. 17/192,538, filed Mar. 4, 2021, the entire contents of each of which are incorporated herein by reference.

### BACKGROUND

The present disclosure relates generally to routing packages. In a computer networked environment such as the internet, users and entities such as people or companies submit network routing requests.

### SUMMARY

In some aspects, the techniques described herein relate to a computer network routing system for routing packages between a computer network originator system and a plurality of computer network processing systems, the computer network routing system including: one or more processing circuits including one or more processors and memory storing instructions that, when executed by the one or more processors, cause the one or more processors to perform operations including: storing, in a network user dataset, network plan information for a plurality of users, the network plan information for at least one user of the plurality of users including a first set of routing attributes; associating, in the network user dataset, a second set of routing attributes with the first set of routing attributes for the at least one user, the second set of routing attributes configured for use by the computer network originator system when submitting the packages; receiving, from the computer network originator system, a package including the second set of routing attributes associated with the at least one user; replacing, for the package, the second set of routing attributes with the first set of routing attributes associated with the at least one user to generate updated routing information corresponding to a computer network processing system of the plurality of computer network processing systems; and sending the package including the updated routing information to the computer network processing system for processing.

In some aspects, the techniques described herein relate to a computer network routing system, wherein the first set of routing attributes includes at least one of an original bank identification number (BIN), an original processor control number (PCN), an original group identifier, and an original member identifier associated with a network plan of the at least one user.

In some aspects, the techniques described herein relate to a computer network routing system, wherein the second set of routing attributes includes at least one of a new BIN, a new PCN, a new group identifier, and a new member identifier associated with a prescription card generated for the at least one user.

In some aspects, the techniques described herein relate to a computer network routing system, wherein: the package including the updated routing information generated by a BIN over BIN translation replacing the second set of routing attributes with the first set of routing attributes includes replacing the new BIN, the new PCN, the new group identifier, and the new member identifier with the original BIN, the original PCN, the original group identifier, and the original member identifier of original network plan information corresponding to the at least one user, the updated routing information corresponding to the computer network processing system of the plurality of computer network routing systems.

In some aspects, the techniques described herein relate to a computer network routing system, wherein the operations further include: responsive to receiving a claim response from the computer network processing system for the package including the updated routing information, generating, without submitting any additional package to any computer network processing system, a notification for the at least one user, the notification including a final cost of a prescribed drug under an network plan associated with the original bank identification number, the original processor control number, the original group identifier, and the original member identifier, and pricing information for at least one lower-cost alternative.

In some aspects, the techniques described herein relate to a computer network routing system, wherein: receiving the package from the computer network originator system includes receiving the package at a claim editor including a claim pre-editor; and replacing the second set pre-editor prior to sending the package to the computer network processing system.

In some aspects, the techniques described herein relate to a computer network routing system, wherein the operations further include: receiving, from the computer network processing system, a claim response responsive to the package including the updated routing information; modifying the claim response to include information identifying the computer network processing system; and sending the modified claim response to the computer network originator system.

In some aspects, the techniques described herein relate to a computer network routing system, wherein: modifying the claim response to include information identifying the computer network processing system includes updating at least one response network segment field response with a network reimbursement identifier associated with the computer network processing system; and modifying the claim response to include information identifying the computer network processing system includes updating at least one response message segment field response with a message indicating a computer network processing system that adjudicated the package.

In some aspects, the techniques described herein relate to a computer network routing system, wherein the operations further include: logging, in a logging dataset, at least one response received from the computer network processing system; generating, for the at least one user associated with the package, a notification including pricing information derived from the claim response received from the computer network processing system; and sending the notification to a user device associated with the at least one user via at least one of a text message, an email message, and a mobile application message.

In some aspects, the techniques described herein relate to a computer network routing system, wherein the pricing information includes at least one of: a patient pay amount for a prescribed drug under an network plan associated with the first set of routing attributes; pricing for one or more alternative prescription plans associated with one or more computer network processing systems; and pricing for one or more discount plans applicable to the prescribed drug; wherein the notification further includes information describing at least one copay assistance program applicable to the prescribed drug based on at least one attribute of the at least one user stored in the network user dataset.

In some aspects, the techniques described herein relate to a computer network routing system, wherein the notification further includes information describing a lower-cost alternative to the prescribed drug, the lower-cost alternative including at least one of: an on-formulary alternative drug; a generic version of the prescribed drug; and a different computer network originator system at which a lower price is available.

In some aspects, the techniques described herein relate to a computer network routing system, wherein the operations further include: obtaining, from one or more data sources, plan data including at least one of discount plan data and network plan data; and storing the plan data in a plan dataset for use in determining at least one of the pricing information and the lower-cost alternative provided to the at least one user.

In some aspects, the techniques described herein relate to a computer network routing system, wherein: storing the network plan information for the plurality of users includes storing, in the network user dataset, the first set of routing attributes including original routing attributes; and associating the second set of routing attributes with the first set of routing attributes for the at least one user includes storing, in the network user dataset, a mapping between the second set of routing attributes including new routing attributes and the first set of routing attributes including the original routing attributes for the at least one user.

In some aspects, the techniques described herein relate to a computer network routing system, wherein: receiving, from the computer network originator system, the package including the second set of routing attributes associated with the at least one user includes intercepting, at the one or more processing circuits, the package from the computer network originator system prior to sending the package to the computer network processing system; and replacing, for the package, the second set of routing attributes with the first set of routing attributes associated with the at least one user includes generating the updated routing information based on the mapping stored in the network user dataset.

In some aspects, the techniques described herein relate to a computer network routing system, wherein: sending the package including the updated routing information to the computer network processing system for processing includes routing, in real time, the package including the updated routing information to the computer network processing system of the plurality of computer network processing systems based on the first set of routing attributes associated with the at least one user; and data transmissions associated with the package and the network plan information are encrypted in transit and data stored in the network user dataset is encrypted at rest.

In some aspects, the techniques described herein relate to a computer network routing system, wherein: storing the network plan information includes receiving the first set of routing attributes from at least one of an employer system and a user device; and associating the second set of routing attributes with the first set of routing attributes includes generating a prescription card for the at least one user including the second set of routing attributes.

In some aspects, the techniques described herein relate to a method, including: storing, by one or more processing circuits in a network user dataset, network plan information for a plurality of users, the network plan information for at least one user of the plurality of users including a first set of routing attributes; associating, by the one or more processing circuits in the network user dataset, a second set of routing attributes with the first set of routing attributes for the at least one user, the second set of routing attributes configured for use by the computer network originator system when submitting the packages; receiving, by the one or more processing circuits from the computer network originator system, a package including the second set of routing attributes associated with the at least one user; replacing, by the one or more processing circuits for the package, the second set of routing attributes with the first set of routing attributes associated with the at least one user to generate updated routing information corresponding to a computer network processing system of the plurality of computer network processing systems; and sending, by the one or more processing circuits, the package including the updated routing information to the computer network processing system for processing.

In some aspects, the techniques described herein relate to a method, wherein the first set of routing attributes includes at least one of an original bank identification number (BIN), an original processor control number (PCN), an original group identifier, and an original member identifier associated with an network plan of the at least one user, and wherein the second set of routing attributes includes at least one of a new BIN, a new PCN, a new group identifier, and a new member identifier associated with a prescription card generated for the at least one user.

In some aspects, the techniques described herein relate to a method, wherein: the package including the updated routing information generated by a BIN over BIN translation replacing the second set of routing attributes with the first set of routing attributes includes replacing the new BIN, the new PCN, the new group identifier, and the new member identifier with the original BIN, the original PCN, the original group identifier, and the original member identifier of original network plan information corresponding to the at least one user, the updated routing information corresponding to the computer network processing system of the plurality of computer network processing systems.

In some aspects, the techniques described herein relate to a computer network routing system for routing packages between a computer network originator system and a plurality of computer network processing systems, the computer network routing system including: one or more processing circuits including one or more processors and memory storing instructions that, when executed by the one or more processors, cause the one or more processors to perform operations including: storing, in a network user dataset, network plan information in a standardized routing format for a plurality of users, the network plan information for at least one user of the plurality of users including a first set of routing attributes in the standardized routing format; providing, over a computer network, remote access to the computer network originator system so that the computer network originator system can submit packages in real time using routing information in a non-standardized format dependent on a hardware and/or software platform of the computer network originator system, the routing information in the non-standardized format including a second set of routing attributes associated with the at least one user; converting, by the one or more processing circuits, the second set of routing attributes in the non-standardized format into the first set of routing attributes in the standardized routing format by replacing, for at least one (e.g., each) received package, the second set of routing attributes with the first set of routing attributes associated with the at least one user to generate updated routing information corresponding to a computer network processing system of the plurality of computer network processing systems; storing, in the network user dataset, the updated routing information for the at least one user in the standardized routing format; and sending, over the computer network and in real time, the package including the updated routing information in the standardized routing format to the computer network processing system for processing.

In some aspects, the techniques described herein relate to a prescription drug claim routing system for routing prescription drug claims between a pharmacy and a plurality of pharmacy benefit managers (PBMs), the prescription drug claim routing system including: one or more processing circuits including one or more processors and memory storing instructions that, when executed by the one or more processors, cause the one or more processors to perform operations including: storing, in a cardholder dataset, insurance plan information for a plurality of users, the insurance plan information for at least one user of the plurality of users including a first set of routing attributes; associating, in the cardholder dataset, a second set of routing attributes with the first set of routing attributes for the at least one user, the second set of routing attributes configured for use by the pharmacy when submitting the prescription drug claims; receiving, from the pharmacy, a prescription drug claim including the second set of routing attributes associated with the at least one user; replacing, for the prescription drug claim, the second set of routing attributes with the first set of routing attributes associated with the at least one user to generate updated routing information corresponding to a PBM of the plurality of PBMs; and sending the prescription drug claim including the updated routing information to the PBM for processing.

In some aspects, the techniques described herein relate to a prescription drug claim routing system, wherein the first set of routing attributes includes at least one of an original bank identification number (BIN), an original processor control number (PCN), an original group identifier, and an original member identifier associated with an insurance plan of the at least one user.

In some aspects, the techniques described herein relate to a prescription drug claim routing system, wherein the second set of routing attributes includes at least one of a new BIN, a new PCN, a new group identifier, and a new member identifier associated with a prescription card generated for the at least one user.

In some aspects, the techniques described herein relate to a prescription drug claim routing system, wherein: the prescription drug claim including the updated routing information generated by a BIN over BIN translation replacing the second set of routing attributes with the first set of routing attributes includes replacing the new BIN, the new PCN, the new group identifier, and the new member identifier with the original BIN, the original PCN, the original group identifier, and the original member identifier of original insurance plan information corresponding to the at least one user, the updated routing information corresponding to the PBM of the plurality of PBMs.

In some aspects, the techniques described herein relate to a prescription drug claim routing system, wherein the operations further include: responsive to receiving a claim response from the PBM for the prescription drug claim including the updated routing information, generating, without submitting any additional prescription drug claim to any PBM, a notification for the at least one user, the notification including a final cost of a prescribed drug under an insurance plan associated with the original bank identification number, the original processor control number, the original group identifier, and the original member identifier, and pricing information for at least one lower-cost alternative.

In some aspects, the techniques described herein relate to a prescription drug claim routing system, wherein: receiving the prescription drug claim from the pharmacy includes receiving the prescription drug claim at a claim editor including a claim pre-editor; and replacing the second set pre-editor prior to sending the prescription drug claim to the PBM.

In some aspects, the techniques described herein relate to a prescription drug claim routing system, wherein the operations further include: receiving, from the PBM, a claim response responsive to the prescription drug claim including the updated routing information; modifying the claim response to include information identifying the PBM; and sending the modified claim response to the pharmacy.

In some aspects, the techniques described herein relate to a prescription drug claim routing system, wherein: modifying the claim response to include information identifying the PBM includes updating at least one response insurance segment field response with a network reimbursement identifier associated with the PBM; and modifying the claim response to include information identifying the PBM includes updating at least one response message segment field response with a message indicating a PBM that adjudicated the prescription drug claim.

In some aspects, the techniques described herein relate to a prescription drug claim routing system, wherein the operations further include: logging, in a logging dataset, at least one received from the pharmacy, the prescription drug claim including the updated routing information sent to the PBM, and the claim response received from the PBM; generating, for the at least one user associated with the prescription drug claim, a notification including pricing information derived from the claim response received from the PBM; and sending the notification to a user device associated with the at least one user via at least one of a text message, an email message, and a mobile application message.

In some aspects, the techniques described herein relate to a prescription drug claim routing system, wherein the pricing information includes at least one of: a patient pay amount for a prescribed drug under an insurance plan associated with the first set of routing attributes; pricing for one or more alternative prescription plans associated with one or more PBMs; and pricing for one or more discount plans applicable to the prescribed drug; wherein the notification further includes information describing at least one copay assistance program applicable to the prescribed drug based on at least one attribute of the at least one user stored in the cardholder dataset.

In some aspects, the techniques described herein relate to a prescription drug claim routing system, wherein the notification further includes information describing a lower-cost alternative to the prescribed drug, the lower-cost alternative including at least one of: an on-formulary alternative drug; a generic version of the prescribed drug; and a different pharmacy at which a lower price is available.

In some aspects, the techniques described herein relate to a prescription drug claim routing system, wherein the operations further include: obtaining, from one or more data sources, plan data including at least one of discount plan data and insurance plan data; and storing the plan data in a plan dataset for use in determining at least one of the pricing information and the lower-cost alternative provided to the at least one user.

In some aspects, the techniques described herein relate to a prescription drug claim routing system, wherein: storing the insurance plan information for the plurality of users includes storing, in the cardholder dataset, the first set of routing attributes including original routing attributes; and associating the second set of routing attributes with the first set of routing attributes for the at least one user includes storing, in the cardholder dataset, a mapping between the second set of routing attributes including new routing attributes and the first set of routing attributes including the original routing attributes for the at least one user.

In some aspects, the techniques described herein relate to a prescription drug claim routing system, wherein: receiving, from the pharmacy, the prescription drug claim including the second set from the pharmacy prior to sending the prescription drug claim to the PBM; and replacing, for the prescription drug claim, the second set of routing attributes with the first set of routing attributes associated with the at least one user includes generating the updated routing information based on the mapping stored in the cardholder dataset.

In some aspects, the techniques described herein relate to a prescription drug claim routing system, wherein: sending the prescription drug claim including the updated routing information to the PBM for processing includes routing, in real time, the prescription drug claim including the updated routing information to the PBM and the insurance plan information are encrypted in transit and data stored in the cardholder dataset is encrypted at rest.

In some aspects, the techniques described herein relate to a prescription drug claim routing system, wherein: storing the insurance plan information includes receiving the first set of routing attributes from at least one of an employer system and a user device; and associating the second set of routing attributes with the first set of routing attributes includes generating a prescription card for the at least one user including the second set of routing attributes.

In some aspects, the techniques described herein relate to a method, including: storing, by one or more processing circuits in a cardholder dataset, insurance plan information for a plurality of users, the insurance plan information for at least one user of the plurality of users including a first set of routing attributes; associating, by the one or more processing circuits in the cardholder dataset, a second set of routing attributes with the first set of routing attributes for the at least one user, the second set of routing attributes configured for use by the pharmacy when submitting the prescription drug claims; receiving, by the one or more processing circuits from the pharmacy, a prescription drug claim including the second set of routing attributes associated with the at least one user; replacing, by the one or more processing circuits for the prescription drug claim, the second set of routing attributes with the first set of routing attributes associated with the at least one user to generate updated routing information corresponding to a PBM of the plurality of PBMs; and sending, by the one or more processing circuits, the prescription drug claim including the updated routing information to the PBM for processing.

In some aspects, the techniques described herein relate to a method, wherein the first set of routing attributes includes at least one of an original bank identification number (BIN), an original processor control number (PCN), an original group identifier, and an original member identifier associated with an insurance plan of the at least one user, and wherein the second set of routing attributes includes at least one of a new BIN, a new PCN, a new group identifier, and a new member identifier associated with a prescription card generated for the at least one user.

In some aspects, the techniques described herein relate to a method, wherein: the prescription drug claim including the updated routing information generated by a BIN over BIN translation replacing the second set of routing attributes with the first set of routing attributes includes replacing the new BIN, the new PCN, the new group identifier, and the new member identifier with the original BIN, the original PCN, the original group identifier, and the original member identifier of original insurance plan information corresponding to the at least one user, the updated routing information corresponding to the PBM of the plurality of PBMs.

In some aspects, the techniques described herein relate to a system, including: one or more processing circuits configured to: store, in a cardholder dataset, insurance plan information for a plurality of users, the insurance plan information for at least one user of the plurality of users including a first set of routing attributes; associate, in the cardholder dataset, a second set of routing attributes with the first set of routing attributes for the at least one user, the second set of routing attributes configured for use by the pharmacy when submitting the prescription drug claims; receive, from the pharmacy, a prescription drug claim including the second set of routing attributes associated with the at least one user; replace, for the prescription drug claim, the second set of routing attributes with the first set of routing attributes associated with the at least one user to generate updated routing information corresponding to a PBM of the plurality of PBMs; and send the prescription drug claim including the updated routing information to the PBM for processing.

### BRIEF DESCRIPTION OF THE DRAWINGS

The present systems and methods for network routing are described in detail below with reference to the attached drawing figures, wherein:

FIG. 1 is a block diagram illustrating a routing system with databases, editors, and networked components, according to some implementation;

FIG. 2 is a block diagram illustrating components of a routing system including an originator domain, a controller with interfaces and allocation, and external data sources and processing systems, according to some implementation;

FIG. 3 is a block diagram illustrating a translation editor interfacing between an originator system and multiple network processing systems with pre-and post-editing stages and associated datasets, according to some implementation;

FIG. 4 is a block diagram illustrating a routing and consumer engagement system showing datasets, interfaces, and processing components for translating prescription identifiers and returning enhanced responses, according to some implementation; and

FIG. 5 is a flow chart illustrating a method for translating routing attributes of packages and forwarding them to a processing system, according to some implementation.

FIG. 6 is a block diagram depicting an example of a claim routing system, and associated environment, according to some implementation;

FIG. 7 is a block diagram depicting an implementation of a claim processing architecture, according to some implementation;

FIG. 8 is a block diagram depicting a claim editor and associated environment, according to some implementation.

FIG. 9 is a block diagram depicting a claim controller, according to an illustrative implementation.

FIG. 10 is a flowchart for a method of routing prescription drug claims, according to some implementation.

FIG. 11 is a block diagram depicting an example of a claim processing workflow in connection with the drug claim processing architecture, according to some implementation.

FIG. 12 is a block diagram illustrating an example computing system suitable for use in the various implementations described herein.

It will be recognized that some or all of the figures are schematic representations for purposes of illustration. The figures are provided for the purpose of illustrating one or more implementations with the explicit understanding that they will not be used to limit the scope or the meaning of the claims.

### DETAILED DESCRIPTION

Below are detailed descriptions of various concepts related to, and approaches, methods, apparatuses, and systems for implementing the various techniques described herein. The various concepts introduced above and discussed in greater detail below can be implemented in any of numerous ways, as the described concepts are not limited to any particular manner of implementation. Examples of specific implementations and applications are provided primarily for illustrative purposes.

This disclosure relates to techniques for translating and routing prescription claim data within distributed computer network environments to provide real-time transparency of cost information for network users. Electronic prescription transactions can occur across interconnected entities, such as pharmacies, insurance networks, and pharmacy benefit managers. These entities rely on structured claim packets that include routing attributes defining how at least one (e.g., each) transaction is directed and adjudicated across computing systems. In typical network implementations, plan and user information can be encoded into identifiers that direct claim traffic over secure channels between originator systems and network processing systems. The routing of such data can involve multiple intermediaries, databases, and communication protocols that collectively form an integrated prescription claim network.

Conventional routing of prescription claims can depend on static mappings between the identifiers on a prescription card and the parameters expected by a corresponding benefit adjudication system. When multiple benefit managers or plan administrators are involved, at least one (e.g., each) can use distinct attributes, such as a bank identification number, a processor control number, a group identifier, or a member identifier. Manual administration of routing settings can introduce latency and risk of misrouting. Systems that cannot update routing mappings in real time can cause claim delays or inaccurate cost reporting. Moreover, users can lack visibility into pricing outcomes at the moment a prescription is processed, limiting the ability to compare costs across plans or identify potential lower-cost alternatives.

The techniques described herein can address these challenges by providing a dynamic claim translation and routing process that maintains a persistent mapping between multiple identifier sets. The techniques can create a new set of routing attributes for at least one (e.g., each) network user and link that set to the original plan identifiers stored in a secure database. When a claim is received from an originator system, such as a pharmacy, the techniques can determine the corresponding original identifiers and generate an updated routing packet directed to the correct network processing system. The translation can occur in real time, thereby maintaining continuity with the original plan configuration while allowing flexibility to modify or layer routing identifiers without disruption to existing adjudication pathways. The techniques can further generate near real-time cost notifications, providing users with information derived from processed claim responses.

In one implementation, a controller can maintain network plan information in a user dataset and associate new attributes with previously stored original attributes. When a routing packet containing new identifiers is received, the controller can access the mapping table to identify the original attribute set. The controller can then substitute the corresponding original values, such that the packet conforms to the routing requirements of the appropriate network processing system. The controller can send the updated packet for adjudication and capture the response returned from the processing system. A notification subsystem can extract cost details from the adjudicated response and deliver a user-facing message through electronic communication channels, such as text or email. Security protocols can encrypt all transmissions in transit and data stored at rest according to privacy regulations.

The techniques described herein can provide several technical improvements. The dynamic routing translation can reduce configuration errors that occur when prescription claim identifiers change across benefit networks. The centralized mapping of identifier sets can maintain routing accuracy while supporting real-time updates from employers or network users. The immediate translation of identifier data during claim submission can decrease processing latency and preserve adjudication accuracy across multiple network connections. By generating a notification based on claim response data, the techniques can offer real-time prescription cost insights without additional transaction overhead. These improvements together can provide significant operational reliability for claim routing systems and increased transparency for network users.

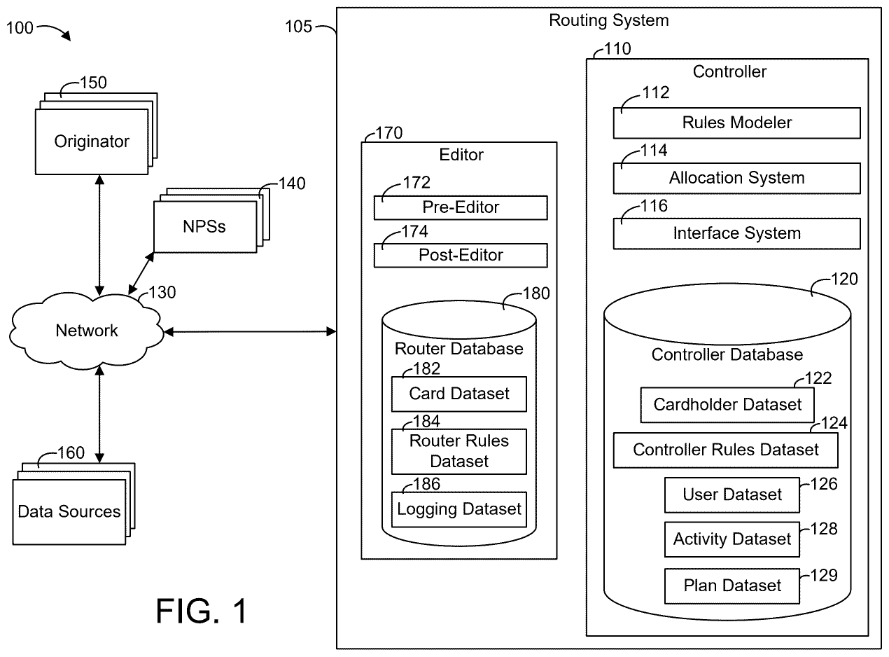

Referring now to FIG. 1, illustrated is a block diagram of a prescription claim routing system 100 for translating and transmitting prescription packet data across networked computing components. The system 100 can include a routing system 105, an originator system 150, one or more network processing systems 140 (also referred to herein as “computer network processing systems” and/or “NPSs”), one or more data sources 160, and a network 130 coupling the components. The routing system 105 can include a controller 110, a controller database 120, and an editor 170. The controller 110 can include a rules modeler 112, an allocation system 114, and an interface system 116. The controller database 120 can include a cardholder dataset 122, a controller rules dataset 124, a user dataset 126, an activity dataset 128, and a plan dataset 129. The editor 170 can include a pre-editor 172, a post-editor 174, and a router database 180. The router database 180 can include a card ID dataset 182, a router rules dataset 184, and a logging dataset 186.

The routing system 105 can be a computer network routing system that facilitates bidirectional transmission of prescription claim packets between an originator system 150, one or more network processing systems 140, and a network 130. The routing system 105 can operate as an intermediary translation layer that modifies routing information embedded within at least one (e.g., each) claim packet to ensure appropriate direction toward a target destination within the network 130. In some implementations, the routing system 105 can perform a substitution process in which a first set of routing attributes related to an original network plan is associated with a second set of routing attributes that correspond to a newly issued prescription card. For example, the routing system 105 can establish a mapping table aligning original identifiers that include a bank identification number, processor control number, group code, and member identifier with corresponding new identifiers that mirror those structural elements. At least one (e.g., each) mapping entry can be created and stored to allow real-time packet translation at transmission time.

In some implementations, the routing system 105 can receive an incoming prescription claim packet transmitted from the originator system 150 using new routing identifiers and apply a translation operation before forwarding the packet to a network processing system (NPS) 140. The routing system 105 can identify the relevant mapping entry in response to the values contained within the inbound packet, retrieve the corresponding original routing attributes, and generate an updated packet in which the original identifiers replace the new identifiers. For example, the routing system 105 can replace a new bank identification number, new processor control number, new group code, and new member identifier with their original counterparts retrieved from a controller database. The translated packet can be prepared in accordance with a prescription benefit manager's routing specification and transmitted toward the adjudication endpoint associated with that benefit manager.

The routing system 105 can coordinate its operations by executing one or more programmed instructions stored in memory to maintain synchronization between translation results and ongoing claim exchange sessions. In some implementations, the routing system 105 can invoke a control process that determines the correct mapping entries based on user identifiers and plan parameters and can apply substitution rules derived from a rules dataset associated with at least one (e.g., each) controller instance. For example, the routing system 105 can generate an execution sequence that retrieves mapping keys from a controller database, performs substitution according to stored conditions, and directs the translated claim toward a defined routing path across the network 130. At least one (e.g., each) controller operation can be executed according to a state transition model that ensures routing attribute replacement occurs before transmission.

The routing system 105 can perform all packet communications through encrypted transmission channels using a Transport Layer Security protocol (TLS 1.2+) and can retain prescription claim data in encrypted storage using the Advanced Encryption Standard (AES-256). In some implementations, the routing system 105 can differentiate between in-transit encryption applied to claim packets exchanged between network nodes and at-rest encryption applied to stored mapping data retained within database files. For example, claim packets transmitted between the routing system 105 and at least one (e.g., each) network processing system 140 (also referred to herein as a “computer network processing system” and/or “NPS”) can be encapsulated using the TLS layer, while the identifier data housed within the associated databases can use AES keys for persistent field-level encryption. Such encryption processes can protect both active and retained identifiers during claim translation and routing across interconnected healthcare payment environments.

The routing system 105 can include a controller 110. The controller 110 can be a distributed processing component that executes operations related to identifier association, rule allocation, and data management within the routing system 105. In some implementations, the controller 110 can generate execution instructions stored in memory that define relationships between routing identifiers and corresponding network plan information used during prescription claim processing. For example, the controller 110 can generate mappings between a new set of identifiers and an original set of identifiers, such as a bank identification number (BIN), processor control number (PCN), group identifier, and member identifier, and record those mappings in the controller database 120. The controller 110 can determine how such mappings are generated and maintained by coordinating operations among the rules modeler 112, the allocation system 114, and the interface system 116. In some implementations, data received through the interface system 116 from an online portal or a batch file upload can trigger the controller 110 to populate the cardholder dataset or update one or more plan datasets. For example, a received enrollment file from an employer system or a user input through a secure portal can cause the controller 110 to assign new identifiers to an individual and link those identifiers to an existing plan record stored in the controller database 120. The controller 110 can apply defined execution rules to maintain bidirectional mappings that align network transactions with both original and newly issued plan identifiers during subsequent claim translation operations.

The controller 110 can include a rules modeler 112. The rules modeler 112 can define, store, and apply rule sets that govern the conditional mapping, transformation, and replacement of routing identifiers processed within the controller 110. In some implementations, the rules modeler 112 can determine one or more substitution rules to convert a set of new identifiers to corresponding original identifiers associated with a user's network plan. For example, the rules modeler 112 can access conditions based on an originating bank identification number (BIN), processor control number (PCN), or group identifier stored in the user dataset and the plan dataset to select a valid mapping rule. In some implementations, the rules modeler 112 can activate validation procedures in the pre-editor 172 prior to transmitting a claim package. For example, the rules modeler 112 can apply instruction sets that verify that the BIN translation and response field alignment comply with established transaction protocols such as those defined by the National Council for Prescription Drug Programs (NCPDP) and Health Insurance Portability and Accountability Act (HIPAA) standards.

The controller 110 can include an allocation system 114. The allocation system 114 can generate, assign, and link routing identifiers to user records stored in the controller database 120. The allocation system 114 can operate according to predetermined mapping rules that define relationships between original and new routing identifiers used during prescription claim transactions. The allocation system 114 can establish at least one (e.g., each) mapping by pairing at least one original bank identification number, processor control number, group identifier, and member identifier with at least one newly generated identifier corresponding to a prescription card. The allocation system 114 can record those pairings in relational tables referenced by the cardholder dataset 122 and accessible for use in subsequently performed claim translation operations.

In some implementations, the allocation system 114 can generate new prescription card identifiers during enrollment or update cycles initiated by external data transmission. For example, a data file transmitted from an employer system or a manual upload performed by a user through an online portal can trigger the allocation system 114 to generate a new set of routing identifiers. The allocation system 114 can assign at least one (e.g., each) identifier set to a unique key that links the new routing identifiers directly to an existing plan record in the cardholder dataset 122. The allocation system 114 can determine the appropriate plan record by matching one or more demographic or membership attributes included in the received data payload.

In some implementations, the allocation system 114 can record the generated linkage information in the plan dataset 129 maintained within the controller database 120. For example, the allocation system 114 can insert association keys into database fields corresponding to the member identifier, group identifier, or processor control number associated with at least one (e.g., each) plan. The allocation system 114 can further cross-reference those keys against mapping tables to preserve relationships between plans, users, and routing identifiers. During live claim processing, the allocation system 114 can perform lookup operations to identify the correct mapping entry corresponding to a specific user and use the entry to supply the controller 110 with matched identifier sets for translation.

The allocation system 114 can generate data fields optimized for rapid retrieval to facilitate high-speed access during real-time claim adjudication. For example, the allocation system 114 can populate indexed data structures such as hash tables or in-memory relational caches that provide constant-time lookup for the routing identifiers. The allocation system 114 can use those indices to identify stored records that match inbound identifiers received in claim packets transmitted by the originator system 150. The allocation system 114 can communicate the matched identifiers directly to the pre-editor 172, allowing the translation operation to access and apply the selected mapping record in real-time processing environments without delay or redundant query execution.

The controller 110 can include an interface system 116. The interface system 116 can facilitate bidirectional data exchange between the routing system 105 and external network entities coupled through the network 130. The interface system 116 can operate as a communication layer that transfers prescription data packets, plan records, and adjudication responses between the routing system 105, the originator system 150, and one or more network processing systems 140. In some implementations, the interface system 116 can establish communication sessions simultaneously with multiple entities to maintain continuous message flow across network links. For example, the interface system 116 can transmit packets through socket-based connections employing defined communication protocols to preserve routing order among concurrent exchanges.

In some implementations, the interface system 116 can receive prescription insurance plan data transmitted by external network entities for processing by the controller 110. The received data can include attribute sets representing bank identification numbers, processor control numbers, group identifiers, and member identifiers associated with different insurance plans. The interface system 116 can parse inbound messages, validate field format conformity according to mapping rules, and forward accepted data to the controller 110 for storage in associated datasets. For example, an employer system can transmit an enrollment file through the interface system 116, and the interface system 116 can parse the file content to isolate identifier fields required for ingestion into the cardholder dataset or plan dataset of the controller database 120.

The interface system 116 can further facilitate bidirectional exchange of prescription claim packets between the routing system 105 and the originator system 150. In operation, the interface system 116 can receive inbound claims from the originator system 150 containing routing attributes associated with newly generated identifiers and forward them to the editor 170 for translation prior to external processing. After adjudication by one or more network processing systems 140, the interface system 116 can retrieve the corresponding responses and return them to the originator system 150. For example, a claim packet transmitted from a pharmacy management terminal can pass through the interface system 116, undergo identifier substitution in the pre-editor 172, and be returned with appended processing identifiers after response enhancement in the post-editor 174.

The interface system 116 can operate according to transport and communication standards compatible with the network 130. In some implementations, the interface system 116 can use secure channel protocols such as Transport Layer Security (TLS) version 1.2 or higher for message transmission between network endpoints. The interface system 116 can manage HTTPS connections initiated by the routing system 105 and maintain session persistence during packet relay operations. For example, a completed adjudication response from a network processing system 140 can be delivered to the originator system 150 through an HTTPS session managed by the interface system 116, wherein the response includes appended metadata identifying the processing system that adjudicated the corresponding claim.

The controller database 120 can be a persistent data store that retains plan mappings, user data, and operational rules used by the controller 110. In some implementations, the controller database 120 can include one or more structured query language servers or distributed cloud repositories that can exchange data through application programming interfaces. For example, the controller database 120 can store network plan information for multiple users such as original and new routing attributes including an original bank identification number, processor control number, group identifier, and member identifier, and their corresponding new forms generated during enrollment. In some implementations, the controller database 120 can maintain encryption at rest using an Advanced Encryption Standard 256-bit algorithm and can record data access timestamps for permitted operations under compliance protocols. For example, the controller database 120 can persist records containing original prescription card identifiers received from employer systems and can flag those records with temporal markers identifying when specific datasets are loaded or updated.

The controller database 120 can include a cardholder dataset 122 that can store structured records representing network users or cardholders to whom prescription cards are issued. At least one (e.g., each) record stored in the cardholder dataset 122 can include data fields for cardholder identifiers, enrollment timestamps, and communication parameters used for subsequent processing across the routing system 105. In some implementations, the controller 110 can generate association pointers referencing the plan dataset 129, user dataset 126, and controller rules dataset 124 to maintain consistent data retrieval during claim translation and adjudication. For example, when the allocation system 114 generates a new set of routing identifiers for an individual user, the allocation system 114 can cause the controller database 120 to update the cardholder dataset 122 by inserting a relational key that links the new identifiers to an existing plan entry in the plan dataset 129. The relational key can be a unique cardholder identifier or a composite key incorporating a member identifier and plan timestamp fields.

In some implementations, the controller 110 can access the cardholder dataset 122 to facilitate cross-component data resolution between the controller 110 and the editor 170 during real-time claim substitution. For example, when the pre-editor 172 receives a claim packet containing new routing identifiers, the allocation system 114 can execute a lookup query on the cardholder dataset 122 to identify the corresponding original routing attributes required to generate the translated packet. The identified attributes can include the original bank identification number, processor control number, group identifier, and member identifier. Once retrieved, the pre-editor 172 can substitute the original attributes in place of the new ones before the interface system 116 transmits the updated packet to the network processing system 140. This cooperative operation among the allocation system 114, the controller database 120, and the editor 170 can facilitate high-speed routing without repeated queries to the external data sources 160.

In some implementations, the controller 110 can use the cardholder dataset 122 in conjunction with the user dataset 126 to retrieve metadata for user-facing communications provided through the engagement interface 220 or other channels. For example, the interface system 116 can access user contact references such as device tokens or secure channel identifiers stored in the user dataset 126 and correlate them with cardholder records in the cardholder dataset 122 to transmit claim outcome notifications derived from processed responses. After the post-editor 174 appends metadata identifying the adjudicating network processing system 140, the interface system 116 can combine the retrieved contact details with the adjudication data to generate a cost notification or alternative drug message. The coordinated operation among the controller 110, the editor 170, and the controller database 120 can maintain synchronized data flows linking cardholder identity, plan mapping records, and real-time claim communication streams.

The controller database 120 can include a controller rules dataset 124 that can store rule sets defining data transformations executed by the controller 110 within the routing system 105. The controller 110, through the rules modeler 112, can determine conditional logic from the controller rules dataset 124 that governs how routing identifiers, metadata, and adjudication parameters are modified or substituted during claim processing. In some implementations, the rules modeler 112 can access relational fields referencing mapping data stored in the cardholder dataset 122 and the plan dataset 129 to determine an appropriate substitution schema based on plan type or network origin. For example, the rules modeler 112 can identify a rule entry specifying a substitution sequence for replacing a new bank identification number with its corresponding original value when a claim is received from an originator system 150 using a particular communication protocol.

In some implementations, the rules modeler 112 can coordinate with the controller rules dataset 124 to dynamically select rule priorities for inbound or outbound transactions. The rules modeler 112 can retrieve stored conditions from the controller rules dataset 124 and instantiate executable instructions defining rule application order and precedence for active claim sessions. For example, the rules modeler 112 can identify instructions indicating that claims adjudicated by certain network processing systems 140 require metadata augmentation before being forwarded through the post-editor 174. The controller 110 can instruct the pre-editor 172 or the post-editor 174 to apply transformation operations that follow the rule hierarchy retrieved from the controller rules dataset 124. This execution path allows the controller 110 to govern multiple mapping rules concurrently across routing operations executed by the routing system 105.

The controller 110 can further access the controller rules dataset 124 when the allocation system 114 generates new prescription card identifiers or when the interface system 116 validates inbound data transmissions. The allocation system 114 can retrieve rule entries that define ranges or conditions for allowable identifier generation to maintain consistency between created routing attributes and stored mapping logic. For example, a rule specifying a permissible range of member identifiers for a plan dataset 129 record can guide the allocation system 114 to generate identifiers within a reserved numeric range. Similarly, the interface system 116 can apply validation conditions defined in the controller rules dataset 124 to verify that inbound enrollment or claim data fields satisfy criteria required for translation. In some implementations, the rules modeler 112 can apply stored transformation instructions that specify metadata fields appended to adjudication responses prior to network transmission, allowing synchronized, rule-driven coordination among the controller 110, the editor 170, and external network entities.

The controller database 120 can include a user dataset 126 that can retain reference information for users whose plan data and outbound notifications are processed by the system 100. The user dataset 126 can store structured records that define user identifiers, communication addresses, and association keys that reference other datasets in the controller database 120. In some implementations, the user dataset 126 can maintain relationships linking at least one (e.g., each) user entry to corresponding cardholder records in the cardholder dataset 122, plan records in the plan dataset 129, and activity records in the activity dataset 128. For example, at least one (e.g., each) user record can include a unique user identifier and a member identifier field that match relational keys stored across those datasets to facilitate coordinated retrieval and correlation of plan data, enrollment information, and notification parameters during controller 110 operations.

In some implementations, the allocation system 114 can access the user dataset 126 to retrieve contact or membership data when generating new prescription identifiers or updating associated plan relationships. For example, when the allocation system 114 generates a new group identifier or member identifier for a prescription card, it can reference the user dataset 126 to confirm that the corresponding user record exists and is aligned with a valid cardholder record in the cardholder dataset 122. The interface system 116 can further access the same user dataset 126 to obtain addresses for outbound communication, such as electronic contact identifiers used for real-time notification delivery through the engagement interface 220. The coordination among the allocation system 114, the interface system 116, and the user dataset 126 can maintain consistency between assigned routing identifiers, plan mappings, and user communication endpoints throughout processing by the routing system 105.

The activity dataset 128 can operate in conjunction with the user dataset 126 to preserve contextual relationships between adjudicated claim records and the corresponding user identifiers maintained in the controller database 120. In some implementations, the controller 110 can use keys stored in the user dataset 126 to correlate an adjudicated claim received via the post-editor 174 with the user from whom the original claim was transmitted through the originator system 150. For example, when a claim is processed by the network processing systems 140 and returned to the routing system 105, the controller 110 can reference the user dataset 126 to align the processed claim data with stored communication preferences and plan relationships before the interface system 116 transmits a notification to the appropriate device. Through these associations, the user dataset 126 can provide the referential framework required for the controller 110 to coordinate plan, identity, and notification data across different components of the system 100.

The activity dataset 128 can be a data repository within the controller database 120 that retains transactional event records associated with real-time prescription claim exchanges performed by the routing system 105. The activity dataset 128 can maintain structured fields identifying elements such as claim identifiers, processing timestamps, network processing system 140 identifiers, and cost result parameters generated during adjudication cycles. The activity dataset 128 can operate in conjunction with other datasets within the controller database 120, including the cardholder dataset 122 and the user dataset 126, which can provide relational keys linking at least one (e.g., each) recorded event to an associated user and plan entry. The activity dataset 128 can thereby represent a data layer through which the controller 110 can access contextual relationships among claims, users, and corresponding plan data during claim translation and response generation.

In some implementations, the controller 110 can access the activity dataset 128 to correlate pre-editor 172 translation operations and post-editor 174 response modifications associated with the same transaction instance. For example, data fields stored in the activity dataset 128 can include a unique transaction identifier generated when the interface system 116 receives a claim packet from the originator system 150. The controller 110 can use this identifier to associate initial mapping references retrieved from the cardholder dataset 122 with final adjudication output parameters originating from a network processing system 140. The interaction between the controller 110 and the activity dataset 128 can facilitate continuous data referencing across the pre-editor 172 and post-editor 174 processing sequences to maintain correct contextual mapping during live claim routing operations.

In some implementations, the interface system 116 can access the activity dataset 128 to retrieve information required for generating output transmissions through other communication interfaces. For example, values stored in the activity dataset 128 can include completion timestamps or response classification codes that indicate when or how a network processing system 140 returned pricing data. The interface system 116 can access those records to trigger preparation of user-facing notifications through the engagement interface 220 or to populate aggregated datasets transferred through the reporting interface 222. The activity dataset 128 can therefore function as a persistent reference store from which the controller 110 and the interface system 116 can retrieve transaction-correlated data required for downstream communication or analytics processes executed within the routing system 105.

The plan dataset 129 can be a data repository within the controller database 120 that retains aggregated network plan information used by the controller 110 during prescription claim translation and adjudication. The plan dataset 129 can store structured records representing plan parameters such as network benefit schedules, formulary tier structures, or pricing relationships among one or more network processing systems 140. In some implementations, the plan dataset 129 can maintain relational links to the cardholder dataset 122 and the controller rules dataset 124 so that the controller 110 can access the appropriate plan attributes when generating substitution mappings or evaluating cost outputs. For example, a relational key stored in the plan dataset 129 can reference a specific group identifier field in the cardholder dataset 122 to correlate a user's coverage attributes with the reimbursement schedule received from external data sources 160.

In some implementations, the allocation system 114 can access the plan dataset 129 to retrieve network plan details during the assignment of new routing attributes or prescription card identifiers. The allocation system 114 can reference applicable benefit levels and discount data stored within the plan dataset 129 to generate consistent mappings between original and new routing identifiers before those identifiers are stored in the cardholder dataset 122. For example, when a new member identifier is created for a user record, the allocation system 114 can locate a matching plan entry within the plan dataset 129 and record the association as part of the relational data maintained by the controller database 120. This relationship allows the controller 110 to interpret plan-specific conditions during later processing steps without modifying or altering the static records present in the plan dataset 129.

The plan dataset 129 can further provide reference information for the interface system 116 and the editor 170 when network plan data is required during claim processing. In some implementations, the interface system 116 can retrieve formulary and pricing information from the plan dataset 129 when responding to data queries from external data sources 160 or network processing systems 140. The editor 170 can use that information through the controller 110 to validate adjudication results appended in a post-editor 174 response before return to an originator system 150. For example, plan dataset 129 entries describing discount program eligibility or co-pay assistance parameters can inform how the controller 110 identifies cost alternatives included in user-facing notifications through the engagement interface 220. The plan dataset 129 therefore provides persistent plan information that is accessed by other active system components to facilitate real-time claim routing and pricing transparency.

The routing system 105 can include an editor 170. The editor 170 can be a data-processing component that performs active modification of prescription claim packets during inbound and outbound data exchanges occurring between the originator system 150 and one or more network processing systems 140. The editor 170 can incorporate multiple sub-processing stages that collectively execute a real-time translation operation referred to as a BIN-over-BIN translation. In some implementations, the editor 170 can identify a second set of routing attributes in an inbound claim packet that correspond to a newly issued prescription card and can replace those attributes with a first set of original routing identifiers retrieved from a mapping table maintained in the controller database 120. For example, the editor 170 can locate a new bank identification number, processor control number, group identifier, and member identifier in a received message and can substitute at least one (e.g., each) with corresponding original fields before relaying the updated data package toward the network processing system 140 through the network 130.

In some implementations, the editor 170 can perform the BIN-over-BIN translation using an indexed lookup operation that references data stored in the card ID dataset 182. The card ID dataset 182 can maintain paired mapping entries associating at least one (e.g., each) new identifier with an original identifier under a one-to-one relationship. For example, a new prescription card record that includes a new BIN and group identifier can maintain a pointer to an original BIN and original group identifier within the same dataset. The editor 170 can access this information during claim ingestion to form a translation key used for immediate substitution before transmission. This mapping process can occur within sub-millisecond latency, thereby maintaining real-time continuity between the originator system 150 and adjudication endpoints across parallel PBM channels. The technical improvement of this arrangement lies in the editor 170's ability to perform the routing translation inline with message flow without requiring batch synchronization or external preprocessing operations.

The editor 170 can employ transformation logic stored in the router rules dataset 184 to determine how at least one (e.g., each) routing attribute substitution occurs across heterogeneous transaction formats. In some implementations, the router rules dataset 184 can specify conditional mapping sequences driven by field priority or protocol version indicators embedded in an inbound claim. For example, the router rules dataset 184 can include transformation scripts specifying that a claim segment identified as NCPDP D.0 can require BIN replacement prior to group code replacement, while another transaction version can require the reverse order. The editor 170 can parse such instructions and execute transformations dynamically as part of a programmable rule engine. This rule-driven workflow can improve protocol interoperability and reduce transmission failures caused by format misalignment or timestamp mismatches encountered in conventional static routing systems.

In some implementations, the editor 170 can perform outbound augmentation operations on claim responses received from one or more network processing systems 140. Upon completion of adjudication, the editor 170 can detect the processing entity that evaluated the claim based on identifiers returned by the PBM and can append supplementary metadata identifying the responsible network processor before returning the response to the originator system 150. For example, the editor 170 can insert a processing-system identifier code and associated reimbursement indicator into specific response segment fields defined by the router rules dataset 184. This post-processing operation can allow the originator system 150 to trace adjudication results without relying on secondary tag matching or external reconciliation scripts. Such packet-level augmentation can provide a technical improvement in response granularity and end-to-end data consistency within distributed transaction environments.

The BIN-over-BIN approach implemented by the editor 170 can provide measurable technical advantages over static routing infrastructures. For example, it can allow updates to plan identifiers without altering downstream processing logic, thereby isolating pharmacy systems from physical data-model revisions. In another, by maintaining the substitution logic within programmable rules accessible by both the pre-and post-editor subsystems, the routing system 105 can execute simultaneous translations across high-throughput claim channels while preserving deterministic timing properties. In yet another example, the inline execution of the translation within the editor 170 can minimize network hops and decrease total packet handling time, resulting in lower latency across adjudication cycles. These improvements collectively contribute to scalable real-time translation performance that enhances computational efficiency, data consistency, and routing precision across interconnected prescription benefit networks.

The editor 170 can include a pre-editor 172. The pre-editor 172 can perform an inbound translation operation that replaces routing identifiers in real time before transmission to a network processing system 140. The pre-editor 172 can determine that an inbound claim packet received from the originator system 150 includes a second set of routing attributes such as a new bank identification number, a new processor control number, a new group identifier, and a new member identifier. In some implementations, the pre-editor 172 can query the controller database 120 to retrieve a first set of routing attributes linked to those identifiers through relational keys stored in a mapping table. For example, the pre-editor 172 can retrieve an original bank identification number, an original processor control number, an original group identifier, and an original member identifier associated with the same user and plan record referenced in the new identifiers. The pre-editor 172 can generate an updated claim packet in which the original identifiers replace the new identifiers to produce revised routing information aligned with the correct adjudication endpoint.

In some implementations, the pre-editor 172 can access translation logic stored in the router rules dataset 184 to determine how and when at least one (e.g., each) routing attribute replacement occurs. The pre-editor 172 can execute ordered substitution sequences that correspond to transaction protocols used by different pharmacy benefit managers. For example, the pre-editor 172 can execute a first substitution sequence for network messages conforming to an NCPDP D.0 transaction specification and a different sequence for a real-time BIN-over-BIN format. The pre-editor 172 can apply translation logic that verifies the positional alignment of substituted fields, such as maintaining the length and checksum of at least one (e.g., each) identifier element to match the expected field structure of the receiving network processing system 140. Through these operations, the pre-editor 172 can ensure that packet structure and data integrity remain consistent with network transmission standards following identifier replacement.

The pre-editor 172 can output the updated claim packet to the network 130 for adjudication after completing all substitutions indicated by the router rules dataset 184. In some implementations, the pre-editor 172 can coordinate with the interface system 116 to transmit the translated packet to one or more network processing systems 140 that correspond to the retrieved original identifiers. For example, the pre-editor 172 can relay the updated packet to a primary processor node when the original bank identification number maps to a specific processor control number assigned to that node. The pre-editor 172 can thereby perform the replacement in real time without additional preprocessing or manual routing configuration. This operational behavior permits seamless translation of inbound claims and preserves compatibility between dynamically generated prescription card identifiers and existing insurance plan routing requirements.

The editor 170 can include a post-editor 174. The post-editor 174 can process claim responses returned from one or more network processing systems 140 after adjudication and before retransmission to the originator system 150. The post-editor 174 can identify a claim response containing reimbursement data, adjudication codes, or plan pricing results and update response segment fields with added information that identifies the specific processing entity. In some implementations, the post-editor 174 can append metadata to the response packet such as a processing-system identifier code or a reimbursement source indicator. For example, the post-editor 174 can write a code into a response message segment field that specifies which network processing system performed adjudication and an accompanying message value describing adjudication status or result type. The post-editor 174 can thus modify outbound data structures to correlate the processed response with the originating processing node.

In some implementations, the post-editor 174 can apply update instructions stored in the router rules dataset 184 to modify response network segment fields according to defined substitution rules. For example, the post-editor 174 can determine offset positions for metadata insertion, overwrite placeholder fields, or allocate extension fields for identifiers that exceed standard segment length. The post-editor 174 can implement such modifications using transformation functions that preserve message formatting required by the NCPDP processing layer while embedding the newly appended metadata. The post-editor 174 can ensure that at least one (e.g., each) updated segment remains aligned with its corresponding inbound transaction reference, such as by preserving the transaction identifier and associated timestamp used during initial claim submission. At least one (e.g., each) applied update can maintain compatibility with subsequent processing steps at the originator system 150 that parse adjudicated results.

The post-editor 174 can forward the modified claim response through the interface system 116 to the originator system 150 after completing all updates. In some implementations, the post-editor 174 can generate a final response structure that combines the network processing system identifier, the adjudication result, and the updated message segment values. For example, the post-editor 174 can insert a response network segment field carrying a reimbursement identifier derived from the processing system and merge that field with other message data before transmission. The post-editor 174 can thereby perform outbound augmentation and field updating in real time, permitting the originator system 150 to receive a response that contains complete adjudication metadata aligned with the translation operations executed by the pre-editor 172. This response-level augmentation maintains continuity between inbound and outbound claim processing within the routing system 105.

The editor 170 can include a router database 180 that can maintain claim translation logic, lookup tables, and routing data accessed by both the pre-editor 172 and the post-editor 174 during live claim processing. In some implementations, the router database 180 can operate as a local or distributed memory system that can be synchronized from controller database 120 to allow real-time access to translation data during message handling. For example, the editor 170 can use the router database 180 to retrieve mapping tables that link new and original routing identifiers and to reference associated transformation rules used during substitution. In some implementations, the router database 180 can facilitate high-speed operations by caching key-value pairs in in-memory structures that can be periodically refreshed from controller database 120 to maintain data consistency with stored plan mappings. For example, a nightly synchronization update can repopulate routing tables and refresh index pointers that the pre-editor 172 and the post-editor 174 use for concurrent lookup and write transactions during claim translation and response augmentation cycles.

The pre-editor 172 and the post-editor 174 can access data stored in the router database 180 as part of their inbound and outbound processing paths. The pre-editor 172 can perform a lookup operation using routing identifiers extracted from an inbound claim to retrieve the corresponding original identifiers stored in the router database 180, and can perform substitution operations according to transformation rules. For example, the pre-editor 172 can request BIN and PCN values from a cached dataset and generate a translated claim packet that the editor 170 transmits to the target network processing system. The post-editor 174 can store translation results and related adjudication parameters into tables maintained within the router database 180 once claim responses are processed. For example, the post-editor 174 can insert outcome metadata or record transaction identifiers to associate at least one (e.g., each) adjudication response with its original claim entry. Through these operations, the router database 180 can serve as the active data repository supporting both substitution and response generation functions by the editor 170.

The router database 180 can include a card ID dataset 182 that can retain numeric and alphanumeric identifiers representing newly issued prescription cards. In some implementations, the card ID dataset 182 can hold records used by the editor 170 to perform real-time cross-referencing during claim translation. For example, when the pre-editor 172 receives a claim including new routing attributes, it can query the card ID dataset 182 through the router database 180 to obtain the corresponding original identifiers used for adjudication. The pre-editor 172 can then generate a translated message that replaces all new identifiers such as BIN, PCN, group, and member identifiers with their original equivalents. In some implementations, the pre-editor 172 can use index keys maintained in in-memory hash tables for sub-millisecond retrieval performance when identifying the original field values required for replacement within inbound messages.

The post-editor 174 can write data back to the card ID dataset 182 to record identifier association results following successful translations or updates received from the controller 110. For example, if the controller database 120 provides an updated mapping for a new prescription card assignment, the post-editor 174 can insert new association entries or modify assigned key pairs within the card ID dataset 182 to preserve synchronization with the main plan record. In some implementations, the editor 170 can employ scheduled or on-demand refresh cycles between the router database 180 and controller database 120 to propagate recent card issuance or revocation data. Such operations can allow the pre-editor 172 to maintain alignment between the lookup mappings used during real-time translation and the authoritative cardholder records, thereby reducing query latency and preventing mismatched routing identifiers within active transmission streams.

The router database 180 can include a router rules dataset 184 that can define executable instructions and transformation procedures applied by the pre-editor 172 and the post-editor 174 during packet processing. In some implementations, the router rules dataset 184 can store conditional mappings and algorithmic sequences describing how and when field substitutions are executed relative to transaction format standards. For example, the pre-editor 172 can access transformation rules from the router rules dataset 184 that specify an ordered sequence for replacing BIN and PCN identifiers based on the inbound transaction header type. The router rules dataset 184 can be implemented as a persistent schema containing stored procedures or transformation scripts that the editor 170 can execute in real time. For example, a SQL-based rule entry can instruct the pre-editor 172 to apply a BIN substitution operation before altering a group identifier field in message formats conforming to a specific standard.

The post-editor 174 can access the router rules dataset 184 to determine field offset locations used for embedding processing-system identifiers into outbound claim responses. For example, after identifying the adjudicating network processing system from response data, the post-editor 174 can retrieve a transformation rule from the router rules dataset 184 specifying where and how to append metadata to the response segment before forwarding it to the originator system 150. In some implementations, the post-editor 174 can follow dynamic rule branching based on the version indicator within the incoming response, thereby ensuring proper alignment of metadata fields during augmentation. The editor 170 can use the router rules dataset 184 as the authoritative reference for rule-driven translation and message restructuring operations that apply across multiple PBM and message protocol variations.

The router database 180 can include a logging dataset 186 that can store records describing inbound and outbound claim packet activity processed by the editor 170. In some implementations, the pre-editor 172 can store entries in the logging dataset 186 following at least one (e.g., each) translation event, capturing details such as transaction identifiers, timestamps, and the mapping keys used for substitution. For example, at least one (e.g., each) claim packet received by the pre-editor 172 can trigger an insertion operation that registers the original and new identifier pairs that were involved in the translation process. The post-editor 174 can insert entries containing response-related metrics such as processing-system identifiers or adjudication result codes upon receiving completed claim responses. In some implementations, the logging dataset 186 can maintain a coherent record of claim transformation and routing outcomes that reflect the operations executed by the editor 170 during live network processing activities.

The post-editor 174 can further reference the logging dataset 186 to retrieve correlation information about previously processed transactions during return communications to the originator system 150. For example, when generating a composite output record or conducting batch synchronization with external reporting services, the post-editor 174 can use stored transaction identifiers from the logging dataset 186 to associate at least one (e.g., each) adjudicated response with its respective inbound claim event. In some implementations, the pre-editor 172 and the post-editor 174 can perform these read and write operations through concurrent accessor threads with isolation controls to maintain data coherency during high-throughput claim routing. The router database 180 can thereby act as a persistent operational repository that facilitates continuous translation, rule-based modification, and transactional reference functionality within the processing scope of the editor 170.

The system 100 can include an originator system 150. The originator system 150 can operate as a primary data transmission endpoint used by a pharmacy or dispensing source to generate and send prescription claim packets across the network 130. In some implementations, the originator system 150 can include computing components such as a pharmacy management terminal, a network interface controller, and a message generation processor that collectively format prescription claim data according to standardized electronic specifications. For example, the originator system 150 can generate a message formatted using a National Council for Prescription Drug Programs (NCPDP) D.0 transaction structure and assign transaction identifiers, routing codes, and timestamp values before transmission. At least one (e.g., each) transmission event initiated by the originator system 150 can package the claim data with routing attributes representing new plan identifiers used for real-time adjudication downstream in the routing system 105.

In some implementations, the originator system 150 can associate user or member information with a second set of network routing attributes that include a newly issued bank identification number, processor control number, group identifier, and member identifier. The originator system 150 can sequence these attributes within a formatted data segment as part of at least one (e.g., each) electronic claim message. For example, the originator system 150 can embed a new BIN value and corresponding PCN in the claim header field, insert the group identifier within the group segment, and map the member identifier within a member segment as instructed by predefined message schema rules. These newly generated identifiers can correspond to a reissued prescription card assigned to a particular user, allowing the routing system 105 to reference a mapping between the new identifiers and original plan identifiers stored in the controller database 120.

The originator system 150 can transmit at least one (e.g., each) formatted claim packet to the routing system 105 over the network 130 using secure and standardized communication protocols. In some implementations, the originator system 150 can initiate an authenticated session employing a Transport Layer Security (TLS) version 1.2 or higher protocol to facilitate encrypted communication during packet transfer. For example, the originator system 150 can open an outbound connection toward a routing endpoint defined by a network address and exchange session keys before sending a claim packet containing the new routing identifiers. The transmitted packet can be received by the interface system 116 within the routing system 105, which can process the claim packet to identify, translate, and forward the data for adjudication. Through these operations, the originator system 150 can function as the network entry point for claim submission, providing the transactional data required for real-time translation and processing by downstream components.

The system 100 can include one or more network processing systems 140 that perform adjudication operations for translated prescription claim packets routed from the routing system 105. At least one (e.g., each) network processing system 140 can evaluate the updated routing identifiers included in a prescription claim to determine eligibility, coverage, and applicable reimbursement according to stored plan data. In some implementations, the network processing systems 140 can correspond to distinct pharmacy benefit managers or adjudication engines linked through the network 130 to process claims associated with multiple healthcare plans. For example, one network processing system 140 can process claims associated with an employer-sponsored plan using a dedicated transaction engine, while another network processing system 140 can adjudicate individual plan claims using different benefit calculation parameters. At least one (e.g., each) network processing system 140 can return a response packet containing adjudication outcomes that identify allowed amounts, member co-pay values, and final reimbursement information for the prescribed medication.

In some implementations, at least one (e.g., each) network processing system 140 can perform its adjudication process using rule-based transactional logic that references benefit configuration datasets. The rule-based transaction engine can parse received claim data, apply eligibility rules, and evaluate member benefit structures based on plan type and routing information. For example, the engine can determine copay percentages, deductible thresholds, or formulary tier relationships for a given prescription claim by evaluating lookup tables stored within the network processing system 140. At least one (e.g., each) rule evaluation can generate output fields that include reimbursement totals for pharmacies and out-of-pocket costs for network users. The rule-based architecture of at least one (e.g., each) network processing system 140 can therefore allow deterministic processing behavior and consistent adjudication across multiple claim sessions originating from the routing system 105.

At least one (e.g., each) network processing system 140 can apply pricing algorithms and reimbursement instruction sets derived from proprietary plan configurations to compute benefit results. In some implementations, the network processing system 140 can determine pricing based on drug identifiers, quantity dispensed, and applicable discount program associations retrieved from its internal databases. For example, the network processing system 140 can identify the reimbursable rate of a prescribed drug by referencing a stored pricing model that defines cost relationships across participating manufacturers or pharmacy contracts. The network processing system 140 can then apply the identified formula to generate adjudication data, which can be transmitted back to the routing system 105 for response enhancement and delivery to the originator system 150. The coordinated operation between the routing system 105 and at least one (e.g., each) network processing system 140 can facilitate accurate and timely computation of benefit results across heterogeneous plan networks.

The system 100 can include one or more data sources 160. The data sources 160 can transmit external data records related to insurance plans, prescription formularies, and discount program schedules toward the routing system 105 for use in adjudication processes. In some implementations, the data sources 160 can provide structured datasets generated by employer systems, pharmacy benefit manager repositories, or manufacturer pricing databases. For example, the data sources 160 can send formatted data packets containing plan cost tables, formulary mappings, or manufacturer discount eligibility information that populate or refresh corresponding records stored in the plan dataset 129. In some implementations, the data sources 160 can send those data packets to the routing system 105 through secure file delivery or application programming interfaces for ingestion into the controller database 120 or the router database 180. For example, periodic file transfers can provide updated formulary cost indices or changes in manufacturer rebate availability, and those records can be parsed by the controller 110 during synchronization cycles to maintain accurate plan mappings across active network users.

The system 100 can include a network 130. The network 130 can be a communication infrastructure that electrically or logically couples the originator system 150, the routing system 105, the network processing systems 140, and the data sources 160. In some implementations, the network 130 can include a set of public and private connectivity channels such as internet-based links, virtual private network tunnels, or dedicated healthcare interconnects. For example, the network 130 can transmit claim packets, cost responses, or plan data updates across the routing system 105 using multiplexed communication sessions executing in real time. In some implementations, the network 130 can carry bidirectional message traffic conveying routing identifiers, pricing information, and user notification data among the connected systems. For example, a translated claim packet generated by the routing system 105 can traverse the network 130 to a network processing system 140 for adjudication and receive a response packet that returns through the same communication channel to the originator system 150. The network 130 can operate according to encrypted transmission standards, such as Hypertext Transfer Protocol Secure (HTTPS) or Secure File Transfer Protocol (SFTP), to maintain confidentiality and integrity of all routing and plan data during transmission.

The controller 110 can maintain in the controller database 120 a first set of routing attributes corresponding to original network plan identifiers assigned to at least one (e.g., each) user. In some implementations, the first set of routing attributes can include an original bank identification number (BIN), an original processor control number (PCN), an original group identifier, and an original member identifier stored in relation to individual plan records in the plan dataset 129. For example, the allocation system 114 can populate the cardholder dataset 122 with data structures that associate at least one (e.g., each) stored identifier set with an appropriate plan record located in the plan dataset 129. The allocation system 114 can execute indexing operations based on key fields such as member identifiers, enrollment timestamps, or card issue numbers that uniquely characterize at least one (e.g., each) user entry. The pre-editor 172 can access the stored attributes derived from the cardholder dataset 122 when processing inbound claim packets originating from the originator system 150 and can use the identified entries to reconstruct the original routing parameters for subsequent message generation.

In some implementations, the controller 110 can apply the mapping records maintained by the allocation system 114 to confirm that at least one (e.g., each) original identifier aligns with an associated network plan entry stored in the plan dataset 129. For example, the controller 110 can execute a selective query that filters records by BIN or PCN values to reference an authoritative plan configuration used by the adjudicating network processing system 140. The pre-editor 172 can retrieve the parameter set from the controller database 120 and replace the new identifiers present in the incoming claim with the corresponding original identifiers before transmission. The updated routing information can incorporate the restored BIN, PCN, group, and member identifiers within appropriate field positions as required by the network processing system 140 to process the adjudication request.

The controller 110 can generate and store in the controller database 120 a second set of routing attributes corresponding to new identifiers assigned to a prescription card generated for at least one (e.g., each) user. In some implementations, the allocation system 114 can populate the cardholder dataset 122 with new BIN, PCN, group, and member identifiers that are mapped directly to their original counterparts in the plan dataset 129. For example, when an employer portal submits a batch enrollment file through the interface system 116, the allocation system 114 can generate the new routing identifiers and create one-to-one linkage records with existing plan entries. The controller 110 can record these pairings in the controller rules dataset 124 to maintain reversible cross-references during future translation operations. The pre-editor 172 can subsequently reference such mapping instructions to determine the proper substitution sequence for at least one (e.g., each) identifier element encountered within a received claim transaction.

In some implementations, the mapping table established in the controller rules dataset 124 can define transformation hierarchies that specify the precedence order among BIN, PCN, group, and member elements during substitution. For example, the controller 110 can associate priority weights with individual attribute fields so that the pre-editor 172 executes substitution operations in a fixed rule sequence appropriate to the formatting constraints of at least one (e.g., each) plan. The allocation system 114 can refresh or extend stored pairings to accommodate newly issued prescription cards by inserting mapping records aligned with corresponding plan dataset 129 entries. The pre-editor 172 can read the mapping index to perform the replacement of new routing identifiers with original identifiers during real-time packet translation for incoming claims.

The editor 170 can execute a BIN-over-BIN translation process that replaces the second set of routing attributes with the first set to produce updated routing information suitable for downstream processing. In some implementations, the pre-editor 172 can perform an indexed lookup in the card ID dataset 182 to identify mapping records linking at least one (e.g., each) new identifier with the corresponding original identifier used by the network processing system 140. For example, when a claim enters the editor 170, the pre-editor 172 can perform key-based retrieval operations to replace at least one (e.g., each) new BIN, PCN, group code, or member ID field with values retrieved from the router database 180. The post-editor 174 can use translation protocols defined in the router rules dataset 184 to maintain consistent message structure for outbound routing. The editor 170 can direct the translated claim through the interface system 116 to the appropriate processing endpoint based on the updated routing information and reference mappings contained within the dataset records.

In some implementations, the pre-editor 172 can process multiple mapping requests in parallel threads to expedite substitution across large-volume transmissions received concurrently from the originator system 150. For example, when a pharmacy sends numerous claims carrying distinct new identifiers, the pre-editor 172 can execute concurrent database access operations to reference all relevant card ID dataset 182 entries without introducing latency. The retrieved original identifiers can replace at least one (e.g., each) new identifier set within their respective packet field segments in accordance with substitution rules stored in the router rules dataset 184. The editor 170 can confirm format alignment before transmitting the translated packet toward the network processing system 140 through the interface system 116 based on the compiled mappings derived from the router database 180.

The interface system 116 can coordinate routing of the updated claim packet to the correct network processing system 140 identified by the corresponding original routing attributes. In some implementations, the interface system 116 can transmit the translated claim packet across the network 130 after receiving confirmation signals from the pre-editor 172 that substitution processing is complete. For example, when a claim addressed to a generic endpoint requires redirection, the interface system 116 can access connection parameters in the plan dataset 129 that specify the destination host for the applicable network plan. The controller 110 can supply correlation data between the restored original BIN and a processing endpoint reference maintained for that BIN in the plan dataset 129. The interface system 116 can forward the revised packet using a routing procedure that selects the appropriate service node without requiring operator configuration updates.

In some implementations, the correlation performed by the controller 110 can include address mapping or lookup operations that link BIN values with connection metadata specific to the network processing system 140. For example, the interface system 116 can identify a corresponding communication socket or endpoint reference derived from a matching BIN record previously stored in the plan dataset 129. The routing process can maintain the initial transaction session initiated by the originator system 150, preventing any reinitialization or lost session identifiers. The coordination among the interface system 116, the editor 170, and the controller 110 can facilitate consistent message relay that preserves alignment between network plan identifiers and adjudication flow paths across the network 130.

The controller 110 can receive a claim response transmitted from the network processing system 140 linked to the updated routing information and can generate a notification referencing the corresponding user. In some implementations, the post-editor 174 can extract cost fields from the returned packet and apply transformation logic defined in the router rules dataset 184 to modify internal response segment data before resubmission to the originator system 150. For example, the post-editor 174 can append metadata describing the network processing system 140 that performed adjudication and can embed a reimbursement identifier or other adjudication indicator into designated message segment positions. The engagement interface 220 within the interface system 116 can generate a message incorporating prescription cost and identified lower-cost alternatives (substitutes) based on the adjudication outcome. The activity dataset 128 can maintain record references linking the processed claim transaction with associated user identifiers used for upcoming notification transmissions.

In some implementations, the modification performed by the post-editor 174 can adjust both content encoding and field positioning to align with defined rules in the router rules dataset 184. For example, the transformation can involve repopulating numerical identifier fields or updating response segment codes to encode metadata identifying which network processing system 140 generated the adjudication response. The controller 110 can use the extracted pricing data to prepare an outbound notification message that includes prescription costs and pricing comparison metrics computed from response parameters. The linkage references stored in the activity dataset 128 can be read during notification generation to correlate the message to the specific user record referenced in the cardholder dataset 122.

The engagement interface 220 can transmit the generated notifications to user devices in near real time using multiple communication formats such as text messages, email, or mobile application messages. In some implementations, the activity dataset 128 can supply targeting identifiers required for communication routing to user-specific endpoints. For example, the controller 110 can query the user dataset 126 to retrieve device tokens, message addresses, or unique identifiers corresponding to recipients associated with adjudicated claims. The controller 110 can assemble outbound transmissions including patient pay amounts, insurance coverage comparisons, and discount rates derived from plan parameters. The interface system 116 can transmit formatted message payloads through the network 130 toward external gateways designated for the selected notification format.

In some implementations, the engagement interface 220 can manage message structure composition using formatting templates aligned with plan-specific configurations recorded within the controller database 120. For example, the engagement interface 220 can determine title fields and numerical precision for price data displayed on handheld device applications relaying prescription results. The coordination among the post-editor 174, the controller 110, and the engagement interface 220 can maintain synchronous message preparation that uses adjudication data as input without reinitiating new claim submissions. The interface system 116 can operate concurrently across multiple transmission types to ensure immediate user notification correlated with adjudication outcomes processed on the network 130.

The post-editor 174 can revise claim response packets by incorporating information that explicitly identifies the network processing system 140 responsible for adjudication. In some implementations, the router rules dataset 184 can include transformation entries defining static offset positions for network segment portions to store identifier metadata and reimbursement codes. For example, the post-editor 174 can use such transformation entries to insert a reimbursement identifier into the response network segment field and to append a message descriptor referencing the adjudicating network processing system 140. The modified response packet can preserve alignment with existing National Council for Prescription Drug Programs (NCPDP) data interchange specifications during outbound delivery. The interface system 116 can send the modified response toward the originator system 150 while maintaining reference to the processing metadata embedded by the post-editor 174.

In some implementations, at least one (e.g., each) transformation instruction contained in the router rules dataset 184 can define parameter validation states or field capacity limits associated with response network segments. For example, the post-editor 174 can parse attributes such as permitted field length, allowable character encoding, and alignment sequence to embed metadata correctly identifying the processing system 140. The system 150 can receive the completed packet containing reimbursement information and descriptive response data without alteration to transaction identifiers used during adjudication. The coordination between the post-editor 174, router rules dataset 184, and interface system 116 can facilitate consistent translation of returned response packets for complete visibility into adjudication results across taxable or non-taxable medication claims.

The cardholder dataset 122 and the activity dataset 128 can be accessed by the controller 110 during message augmentation to maintain correlation between processed claims and associated user records. In some implementations, the controller 110 can perform reference lookups to associate a transaction identifier with the relevant cardholder data retrieved from the cardholder dataset 122. For example, when reimbursement outcomes are retrieved from a network processing system 140, the post-editor 174 can use mapping keys in the logging dataset 186 to align output packet updates with originating claim markers stored in the controller database 120. The resulting message can maintain data field consistency across all cost or adjudication-related parameters linked to a single transaction instance. The originator system 150 can receive reconciled responses incorporating field extensions that present adjudicator-specific codes tied to active prescription cost metrics.

In some implementations, the activity dataset 128 can preserve transactional associations by indexing user identifiers to adjudication timestamps and corresponding claim inputs. For example, after identifying the transaction entry, the controller 110 can confirm correct correlation before post-editor 174 fields are updated in the output response packet. The packet returned to the originator system 150 can convey accurate adjudication mapping that references the same network processing system 140 identified earlier within the controller database 120. The interaction among the cardholder dataset 122, the activity dataset 128, and the post-editor 174 can maintain continuous association between adjudicated results and the electronic claim mappings used for routing within the routing system 105.

The controller 110 can calculate pricing components derived from response segment data provided by the network processing system 140 and generate user notifications that reference available cost alternatives. In some implementations, the controller 110 can query the plan dataset 129 to retrieve cost formulas, rebate coefficients, and reimbursement factors obtained from the data sources 160. For example, upon receiving claim response data, the controller 110 can apply stored pricing functions to determine patient pay amounts or to calculate comparative cost outputs across alternative plan types. The adjusted results can be used to specify recommended substitute products or plans within a message payload produced by the engagement interface 220. The payload can include numeric price values, applicable coverage ranges, and benefit classifications aligned with corresponding formulary identifiers stored in the plan dataset 129.

In some implementations, the computed data prepared by the controller 110 can be transferred to the engagement interface 220 for message assembly without additional processing by the network processing system 140. For example, the engagement interface 220 can include data structures labeling discount-program eligibility or manufacturer rebate participation obtained through previous plan synchronization. The formatted message payload can incorporate these variables to denote available savings tied to the medication tier recorded in the plan dataset 129. The engagement interface 220 can generate a display-ready record containing calculated amounts and descriptive text for patient-facing applications or email templates associated with post-adjudication notifications.

The interface system 116 can transmit message payloads generated by the controller 110 through user-specific delivery paths defined in the user dataset 126. In some implementations, the interface system 116 can route notifications using communication modes such as text, email, or mobile interface delivery depending on endpoint data stored for at least one (e.g., each) user. For example, corresponding entries in the user dataset 126 can specify one or more identifiers linking to third-party gateways utilized for outbound communication by the system 116. At least one (e.g., each) notification can include cost values derived from claim response data, program participation indicators, or comparative medication pricing data sourced from the plan dataset 129. The interface system 116 can relay compiled content through network 130 transmission channels designated for at least one (e.g., each) communication type.

In some implementations, the engagement interface 220 can execute formatting operations to convert numeric pricing outputs and assistance program identifiers into structured message elements suited for the recipient device format. For example, a text message output can include concise pricing and co-pay details while email communication can contain expanded data segments describing available savings programs or formulary positioning. The engagement interface 220 can execute sequencing and field-rendering instructions retrieved from the controller database 120 to preserve presentation order across varying message layouts. The message generation workflow can correlate at least one (e.g., each) notification to a transaction identifier linking adjudicated cost data to the corresponding patient and plan record stored in the plan dataset 129.

The controller 110 can access the plan dataset 129 to obtain plan data and discount attributes that determine the content of cost-based notifications delivered to users. In some implementations, the plan dataset 129 can store structured network plan data such as coverage tier matrices, reimbursement multipliers, and benefit schedules received from data sources 160. For example, the controller 110 can issue query operations that extract formulary identifiers and rebate coefficients relevant to a prescription claim, generating applied pricing fields for the notification prepared through the engagement interface 220. The retrieved plan parameters can include cost-sharing values, deductible thresholds, or preferred pharmacy designations that affect the comparative prices reported to the user device.

In some implementations, the allocation system 114 can maintain synchronization of plan data within the plan dataset 129 by performing updates at predetermined intervals based on received pricing inputs. For example, the allocation system 114 can align stored discount plan tables with manufacturer rebate files or insurer rate adjustments sourced from the data sources 160. During claim processing, the controller 110 can cross-reference the synchronized plan records with mapping data maintained in the cardholder dataset 122 to identify which benefit plan applies to at least one (e.g., each) incoming claim. The engagement interface 220 can then compile cost and alternative drug data based on the correlated plan entry, transmitting an outbound notification through the network 130 that reflects current price schedules and available lower-cost alternatives (options). The transmitted information can use value ranges recorded in the plan dataset 129 to accurately represent active discount programs identified for the user.

The controller 110 can maintain, in the controller database 120, a mapping between a first set of routing attributes representing original identifiers and a second set of routing attributes representing new identifiers for at least one (e.g., each) user. In some implementations, the mapping can be retained in relational fields of the user dataset 126 referencing records from the cardholder dataset 122 and the plan dataset 129. For example, the controller 110 can generate a unique key pair that links a new bank identification number and a new processor control number to a corresponding original pair for a specific plan. The allocation system 114 can create the keys when enrollment files are imported from employer systems or during user updates received through the interface system 116. These associations can be maintained to allow immediate retrieval of original routing identifiers during real-time claim translation.

In some implementations, the pre-editor 172 can access the mapping created by the allocation system 114 through query operations to the router database 180 to perform synchronized translation of routing identifiers during claim interception. For example, the pre-editor 172 can apply a lookup sequence using the mapped key set to identify which original identifiers should replace corresponding new identifiers in an inbound transaction. At least one (e.g., each) relational mapping can include identifiers for the plan entry and the member record used during claim adjudication by the network processing system 140. The controller database 120 can maintain bidirectional mapping consistency between user enrollment data and real-time translation logic executed by the pre-editor 172.

The pre-editor 172 can intercept inbound claim packets transmitted from the originator system 150 and perform translation operations to align the packet with correct network routing information. In some implementations, the pre-editor 172 can extract the new bank identification number, processor control number, or group identifier fields contained within the transaction header and use them as input parameters to a lookup function executed on the router database 180. For example, the pre-editor 172 can resolve an original identifier set retrieved from the card ID dataset 182 and update the packet header in real time with the corresponding values. The updated claim packet can maintain all non-routing fields from the original message while replacing only routing-specific attributes, facilitating compatibility with adjudication systems downstream in the network 130.

In some implementations, the interface system 116 can forward at least one (e.g., each) translated claim packet produced by the pre-editor 172 for adjudication by one or more network processing systems 140. The interface system 116 can determine the transmission endpoint by evaluating address references associated with the original routing attributes stored in the controller database 120. For example, when the translated packet includes an original processor control number associated with a particular plan administrator, the interface system 116 can initiate outbound routing toward that processing node without reinitiating the transaction stream. The substitution and immediate forwarding operations can maintain continuity of claim exchange between newly issued prescription identifiers and original plan routing parameters across the network 130.

The interface system 116 can transmit updated claim packets generated by the pre-editor 172 in real time to the network processing system 140 identified by the controller 110. In some implementations, the interface system 116 can determine a routing path based on mapping data stored in association with the original network attributes. For example, the interface system 116 can reference a connection table maintained in the controller rules dataset 124 to translate an original bank identification number into a corresponding processing node address. The interface system 116 can apply transport handling logic that assigns delivery parameters such as session identifiers or packet transfer priorities to the outbound claim packet. At least one (e.g., each) data exchange can occur through communication channels established over the network 130 using high-throughput network protocols. The controller database 120 can store network plan records used to identify the permissible routing endpoints associated with the claim translation performed by the pre-editor 172.

In some implementations, the interface system 116 can monitor connection state information to maintain stream continuity during simultaneous transmissions between multiple network processing systems 140. The interface system 116 can determine parallel channel utilization policies and can select an available channel based on transaction queue size and routing priority. For example, a processing node linked to a specific original processor control number can be assigned a dedicated socket connection, whereas packets sharing a common group identifier can be multiplexed through a shared session. The mapping data accessed by the controller 110 can define allocation limits or routing precedence among multiple paths listed in the controller rules dataset 124. The interface system 116 can repeatedly exchange message acknowledgments with at least one (e.g., each) processing system to maintain sequence alignment during adjudication message flow across the network 130.

The allocation system 114 can receive network plan attributes from an external employer system or user device and generate prescription card identifiers corresponding to those attributes. In some implementations, the allocation system 114 can parse inbound enrollment data transmitted through the interface system 116 and extract identifiers for at least one (e.g., each) user or plan. For example, the allocation system 114 can isolate original bank identification numbers, processor control numbers, group identifiers, or member identifiers contained in a batch upload file. The allocation system 114 can apply generation logic that produces new routing identifiers aligned with the original dataset fields and record those identifiers within the plan dataset 129. At least one (e.g., each) association between original and new routing identifiers can be represented as a row in a relational mapping table stored within the controller database 120 and accessible to the controller 110 during subsequent translation operations.

In some implementations, the allocation system 114 can validate the integrity of at least one (e.g., each) identifier pair before storing the mapping record. The validation can involve cross-referencing demographic identifiers or membership details provided in the inbound file against existing records in the cardholder dataset 122. For example, if an inbound record includes a member identifier not currently represented in the dataset, the allocation system 114 can append a new plan entry and generate a distinct routing identifier set. The allocation system 114 can transmit the newly generated prescription card record through the interface system 116 to the originator system 150 using the network 130. At least one (e.g., each) transmitted record can comprise new routing attributes correlated with the original identifiers that were extracted from the enrollment dataset.

The controller 110 can finalize prescription card generation by synchronizing data maintained across the cardholder dataset 122, the user dataset 126, and the plan dataset 129. In some implementations, the allocation system 114 can insert newly generated routing identifiers into the cardholder dataset 122 and record cross-references linking at least one (e.g., each) new identifier with its originating plan record. For example, when an employer enrollment file triggers the creation of new routing identifiers, the allocation system 114 can generate mapping keys that reference the original identifier record. The controller 110 can invoke the rules modeler 112 to determine applicable conditions governing the relational updates. The rules modeler 112 can apply execution rules stored in the controller rules dataset 124 to verify that all identifiers correspond to the correct user record in the user dataset 126 prior to synchronization. The result of the synchronization can maintain state consistency among datasets within the controller database 120.

In some implementations, the controller 110 can perform iterative checks across dataset keys to ensure mapping coherence between routing identifiers generated by the allocation system 114 and translation sequences executed by the pre-editor 172. For example, an updated plan record stored in the plan dataset 129 can contain composite keys referencing both the original and new plan identifiers, which the rules modeler 112 can cross-validate before publication to the router database 180. The controller 110 can confirm that the association integrity permits correct retrieval of mapping data during a claim translation operation. The synchronization among these datasets allows the controller 110 to maintain data relationships necessary for real-time identifier substitution performed during prescription claim adjudication.

Generally, the routing system 105 can implement operations corresponding to storage, association, translation, and routing of prescription claim data. The controller 110, through the allocation system 114, can store insurance plan information for multiple users in the cardholder dataset 122 of controller database 120, where at least one (e.g., each) record includes a first set of routing attributes such as an original bank identification number, processor control number, group identifier, and member identifier. These stored values represent the foundational network plan information used during claim adjudication by one or more pharmacy benefit managers (PBMs).

The routing system 105 can further associate, in the cardholder dataset 122, a second set of routing attributes with the first set for at least one (e.g., each) stored user record. This second set can correspond to newly generated identifiers assigned during prescription card creation and configured for use by a pharmacy originator system 150 when submitting prescription drug claims. The allocation system 114 can record the mapping between the new and original identifiers, establishing a one-to-one relationship within the controller database 120. When the pharmacy transmits a claim through network 130, the pre-editor 172, operating within the editor 170, can intercept the inbound claim containing the new routing identifiers and locate the associated original identifiers in the mapping table stored in the cardholder dataset 122.

Upon receiving the prescription drug claim from the pharmacy originator system 150, the pre-editor 172 can replace the second set of routing attributes with the first set to generate updated routing information that directs the claim to the correct PBM among the network processing systems 140. The substitution process can utilize transformation logic stored in the router rules dataset 184 to ensure that field alignment and transaction integrity match processing requirements defined by at least one (e.g., each) PBM. The interface system 116 can then transmit the translated claim through network 130 toward the corresponding PBM for adjudication. This sequence of storing, associating, receiving, replacing, and sending allows the routing system 105 to maintain continuous, rule-driven network interoperability and ensure accurate claim adjudication routing.

Generally, the routing system 105 can operate as an integrated translation and transmission framework that electronically links the controller 110, interface system 116, editor 170, and router database 180 with the originator system 150, the network processing systems 140, and the data sources 160 through the network 130. The controller 110 can manage storage of network plan information for multiple users in the controller database 120, including, for at least one (e.g., each) user, a first set of routing attributes arranged in a standardized routing format conforming to predetermined benefit transaction specifications. The stored standardized records can include structured data fields representing an original bank identification number, processor control number, group identifier, and member identifier related to at least one (e.g., each) user's network plan configuration. The controller database 120 and its corresponding user dataset can maintain such data as normalized routing entries used for consistent adjudication processing.

The interface system 116 can provide remote access through the network 130 that enables the originator system 150 to submit electronic packages in real time using routing information expressed in a non-standardized format. The non-standardized routing format can vary according to the originator system's local hardware and software platform, reflecting data element positioning or field structure that differ from the stored standardized format. At least one (e.g., each) package received in this non-standardized format can contain a second set of routing attributes associated with at least one user record managed by the controller 110. The interface system 116 can validate the received data and forward it to the editor 170 for conversion into the standardized routing format required by the network processing systems 140.

The editor 170, operating as part of the routing system 105, can convert the second set of routing attributes in the non-standardized format into the first set of routing attributes in the standardized routing format. The pre-editor 172 can identify within at least one (e.g., each) package incoming from the originator system 150 one or more non-standard routing elements and replace them with corresponding standardized routing identifiers retrieved from the controller database 120. The replacement process can generate updated routing information that aligns with the routing specifications of the appropriate network processing system 140. The updated information can then be stored in the controller database 120 user dataset as standardized entries to maintain continuity in adjudication processing and audit traceability.

The interface system 116 can then transmit, over the network 130 and in real time, at least one (e.g., each) package containing the updated routing information in the standardized routing format to a corresponding network processing system 140 for processing. The post-editor 174 can confirm compliance of the outbound data with rule sets defined in the router rules dataset 184 and can update logging data within the logging dataset 186 to reflect transaction identifiers and processing timestamps. This cooperative operation among the controller 110, editor 170, interface system 116, and router database 180 enables the routing system 105 to maintain accurate bidirectional translation between non-standardized submission formats and standardized routing formats, ensuring that at least one (e.g., each) prescription claim is stored, converted, and transmitted in real time within a uniform data framework.

The controller 110 can include an artificial intelligence model and/or machine learning model that can apply one or more trained models to analyze routing attributes and plan relationships during claim translation operations performed by the routing system 105. In some implementations, the artificial intelligence model and/or machine learning model can employ one or more machine learning models, such as supervised learning models, neural network models, or deep neural network models, to generate predictive outputs that optimize routing accuracy based on learned transaction patterns. For example, the artificial intelligence model and/or machine learning model can receive routing identifiers, plan metadata, or claim payload features as input signals and can output mapping predictions indicating which network processing systems 140 correspond to a given identifier set. In some implementations, the artificial intelligence model and/or machine learning model can incorporate rule-based heuristics or parameterized functions with its trained model layers to correct outlier cases or to refine predictions according to plan-specific constraints.

In some implementations, the artificial intelligence model and/or machine learning model of controller 110 can execute during inbound claim translation, outbound response augmentation, or training cycles that rely on archived claim data. The model can perform forward propagation of input vectors through multiple layers composed of weighted nodes and activation functions to produce probabilistic classification outputs. For example, the artificial intelligence model and/or machine learning model can process claim features such as claim amount, processor control number, or network identifier embeddings and can generate confidence scores correlating to PBM routing decisions. The controller 110 can use the generated scores to refine or select alternate routing paths that minimize adjudication latency across network 130. In some implementations, the artificial intelligence model and/or machine learning model can reduce dimensionality of feature vectors using embedded attention layers, enabling faster inference while maintaining alignment of routing field positions with stored plan attributes.

In some implementations, the artificial intelligence model and/or machine learning model can maintain model training parameters in non-transitory storage for iterative optimization through techniques such as gradient descent and backpropagation applied across batched data subsets derived from historical claim mappings. For example, the artificial intelligence model and/or machine learning model can determine loss values representing differences between predicted network destinations and validated adjudication outcomes and can repeatedly update weight parameters to minimize the loss function. The artificial intelligence model and/or machine learning model can apply dropout regularization to prevent overfitting to specific BIN-PCN combinations and can execute training on distributed processors such as GPUs or TPUs integrated with the routing system 105 to increase computational throughput during large-scale optimization sessions. In some implementations, the artificial intelligence model and/or machine learning model can perform inference without retraining by loading pre-calibrated weight matrices into memory at runtime to evaluate new claim packets transmitted by originator system 150.

In some implementations, the artificial intelligence model and/or machine learning model can evaluate trained models based on defined performance metrics such as precision, recall, and F1 score derived from validation datasets stored in the controller database 120. For example, the artificial intelligence model and/or machine learning model can generate a confusion matrix identifying classification correctness between selected routing endpoints and expected destinations as recorded during previous adjudication sessions. The artificial intelligence model and/or machine learning model can compare measured accuracy values against predetermined threshold criteria to determine whether an updated model achieves acceptable inferencing reliability before deployment. In some implementations, the artificial intelligence model and/or machine learning model can perform cross-validation and Monte Carlo sampling procedures to test model robustness against noise in historical transaction distributions. Evaluation results can determine whether model parameters require adaptive retraining to maintain accuracy in operational claim translation environments.

The artificial intelligence model and/or machine learning model can include one or more neural network models arranged into an input layer, one or more intermediate layers, and an output layer, at least one (e.g., each) containing nodes spanning feature dimensions of claim routing parameters. In some implementations, the input layer can receive numeric or categorical embeddings representing routing identifiers or claim field attributes, and the output layer can produce classification vectors specifying routing targets or substitution profiles. For example, intermediate layers can compute weighted combinations of embeddings to model nonlinear dependencies between network identifiers and plan parameters retrieved from the plan dataset 129. The resulting output can indicate routing assignment probabilities that the controller 110 can apply to dynamically select an optimal network processing system 140 during packet adjudication.

The system 100 can execute model training and tuning procedures for the artificial intelligence model and/or machine learning model during data ingestion or operational claim exchanges. In some implementations, backpropagation updates performed by the controller 110 can modify weight and bias values responsive to loss gradients evaluated on batches of training examples that include historical claim outcomes and corresponding routing configurations. For example, at least one (e.g., each) iteration of the training cycle can adjust neural layer coefficients when the model predicts an incorrect mapping between a new identifier set and an original routing identifier set. In some implementations, the artificial intelligence model and/or machine learning model can train on aggregated data describing multiple plan types or network segments and can apply transfer learning to generalize learned routing behaviors across varied pharmacy benefit manager infrastructures.

In some implementations, the controller 110 can apply a pre-training stage for the artificial intelligence model and/or machine learning model using unstructured or pseudo-labeled datasets to develop generic feature embeddings that represent routing relationships among identifier pairs without plan-specific supervision. For example, the artificial intelligence model and/or machine learning model can perform self-supervised learning through predictive masking objectives on partial transaction sequences or autoencoder-based reconstruction of missing identifiers. The pre-trained weight parameters can initialize downstream models adapted for real-time translation of pharmacy claim packets. In some implementations, pre-training can include distributed processing using data parallelism or model parallelism to segment high-dimensional input matrices across multiple compute nodes, followed by parameter aggregation at synchronization intervals.

The controller 110 can implement a fine-tuning phase during which the artificial intelligence model and/or machine learning model updates pre-trained representations to specialize in plan-specific mapping and translation of identifiers. In some implementations, the model can perform supervised learning on labeled routing datasets containing verified associations between new and original identifiers. For example, the artificial intelligence model and/or machine learning model can apply a gradient-based optimization procedure using labeled samples extracted from validated adjudication records and can terminate fine-tuning when validation accuracy converges within a specified range. In some implementations, the controller 110 can employ low-rank adaptation techniques or adapter layers that adjust selected matrix subsets in the neural architecture to reduce computational costs while retaining learned generalization capacity across multiple plan types.

In some implementations, the controller 110 can employ a retrieval-augmented generation model in conjunction with the artificial intelligence model and/or machine learning model to reference external datasets stored in the plan dataset 129 or data sources 160. The retrieval-augmented generation model can include a retrieval system that can identify relevant historical mappings and a generation system that can synthesize new substitution instructions derived from retrieved results. For example, the retrieval component can perform vector search using approximate nearest neighbor comparison to identify prior transactions aligned with current claim features, and the generative component can output substitution sequences that the pre-editor 172 can apply during routing translation. In some implementations, retrieval parameters can be adjusted dynamically based on the complexity or ambiguity of inbound claim attributes to maintain balanced computational efficiency and prediction quality.

In some implementations, the controller 110 can include a sparse expert-based model architecture that incorporates a mixture-of-experts configuration within the artificial intelligence model and/or machine learning model. The mixture-of-experts configuration can include multiple expert subnetworks specialized for different types of routing transformations, such as BIN substitution, PCN translation, or group code inference, among others. A gating component can determine which subset of experts to activate for a given input vector representing an inbound claim. For example, when the artificial intelligence model and/or machine learning model processes a high-volume claim category associated with a complex plan mapping, the gating mechanism can activate experts optimized for multi-tier formulary substitution. In some implementations, the sparse expert-based architecture can employ multi-head attention to maintain alignment between long-range identifier dependencies and can apply tensor parallelism to distribute inference tasks across processing units within the routing system 105 for real-time claim translation.

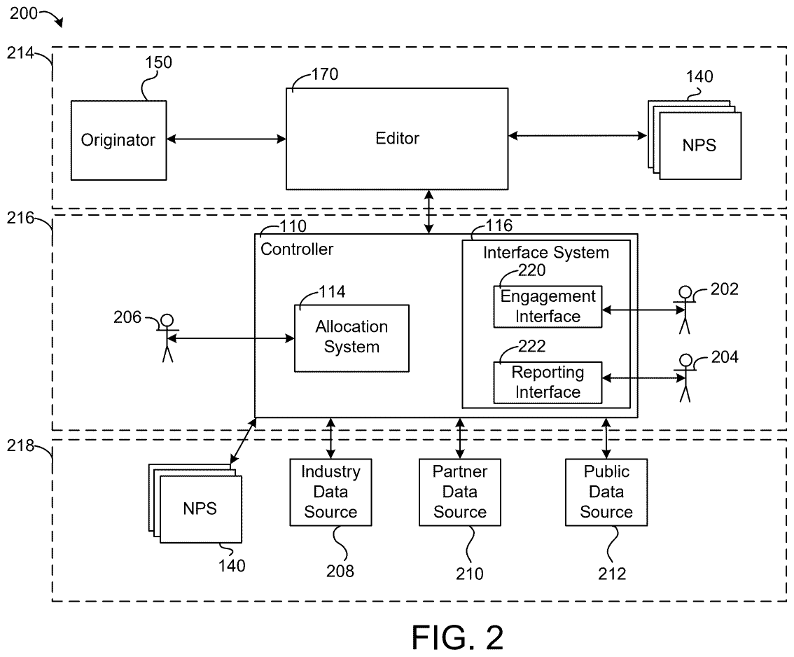

Referring now to FIG. 2, illustrated is a block diagram of a system 200 depicting an example prescription drug claim routing system with multiple operational domains according to one or more implementations. The system 200 can include an originator zone 214, a controller zone 216, and a data and processing zone 218. The originator zone 214 can include an originator system 150 and an editor 170. The controller zone 216 can include a controller 110, which can include an allocation system 114 and an interface system 116. The interface system 116 can include an engagement interface 220 and a reporting interface 222. The data and processing zone 218 can include one or more network processing systems 140, and one or more external data sources such as an industry data source 208, a partner data source 210, and a public data source 212. The controller zone 216 can communicate with one or more users 202, 204, and 206 through respective input or data transmission channels.

The originator zone 214 can include one or more computing entities that generate and transmit prescription claim data toward network-connected processing nodes. The originator zone 214 can operate as an initial data entry layer through which a pharmacy originator system 150 communicates claim packets to an editor 170 for subsequent translation and routing. In some implementations, the originator zone 214 can include communication interfaces, data formatting engines, and transaction initiators that prepare claim data according to standardized message formats. For example, the originator zone 214 can transmit claim packets coded with new routing identifiers such as bank identification numbers or processor control numbers aligned with prescription card records generated by a controller 110. In some implementations, the originator zone 214 can maintain secure network sessions with the controller zone 216 to facilitate real-time bidirectional exchange of claim data, adjudication responses, or transaction metadata exchanged between an originator system 150 and an editor 170. The originator zone 214 can include an originator system 150. The originator zone 214 can include an editor 170.

The system 200 can include a controller zone 216 that operates as a central processing environment for coordinating data transactions, routing instructions, and network plan management among the connected zones. In some implementations, the controller zone 216 can include hardware and software components that determine mapping relationships between original routing identifiers and new routing identifiers stored within system datasets. For example, the controller zone 216 can access plan and cardholder information through controller 110, allocation system 114, and interface system 116 to generate routing instructions that define how claim packets are transmitted to network processing systems 140. In some implementations, the controller zone 216 can execute control processes that regulate inbound and outbound data flow between the originator zone 214 and the data and processing zone 218 through secure communication protocols. For example, the controller zone 216 can receive claim packets from an editor 170, evaluate substitution conditions derived from stored transformation rules, and issue packet redirection commands to align routing information with corresponding processing endpoints across network 130.

The controller zone 216 can include a controller 110 that operates as a primary coordination entity for data routing, mapping, and communication processing throughout system 200. The controller 110 can include an allocation system 114 that determines relational associations between user-specific prescription identifiers and network plan records stored in one or more datasets accessible to the controller zone 216. The controller 110 can further include an interface system 116 that transmits and receives network messages associated with user enrollment, claim adjudication, and notification delivery. In some implementations, the interface system 116 can include an engagement interface 220 that exchanges user-originated or system-originated data streams in real time through network 130. For example, the engagement interface 220 can send outbound responses and receive acknowledgment messages associated with adjudication transactions or informational display events initiated by external user devices coupled to system 200.

The engagement interface 220 can function as a transaction-terminal endpoint that provides multiple notification channels for individual users 202 and/or 204 following the completion of claim processing operations. In some implementations, the engagement interface 220 can deliver prescription cost notifications and alternate pricing messages through short-message service, electronic mail, and/or mobile application transmissions. For example, after a network processing system 140 returns adjudicated cost data, the engagement interface 220 can generate a data message containing the patient pay amount, copay estimation, or availability of a lower-cost medication substitution (e.g., lower-cost alternatives). In some implementations, the engagement interface 220 can exchange data packets over encrypted transport sessions using Transport Layer Security (TLS) version 1.2 or higher, and can append authentication tokens to outbound headers. For example, the engagement interface 220 can execute secure application programming interface (API) calls toward third-party communication gateways that meet Health Insurance Portability and Accountability Act (HIPAA) and National Council for Prescription Drug Programs (NCPDP) compliance requirements to maintain protected transmission paths for all reported pricing data.

The interface system 116 can include a reporting interface 222 that provides a data acquisition endpoint for administrative or analytical operations performed within controller zone 216. The reporting interface 222 can access linked datasets such as the activity dataset 128 or plan dataset 129 and generate aggregate data structures representing adjudication outcomes across multiple network processing systems 140. In some implementations, the reporting interface 222 can synthesize adjudication counts, reimbursement averages, or response-time statistics by issuing query requests to internal data tables. For example, the reporting interface 222 can produce dataset outputs containing average prescription cost trends across pharmacy benefit managers, discount rate utilizations, or reimbursement source distributions. The reporting interface 222 can facilitate data presentation for visualization tools without altering the stored adjudication records.

In some implementations, the reporting interface 222 can execute scheduled query operations that extract claim timestamps and derived metrics from the controller database 120 for compliance and audit aggregation. For example, the reporting interface 222 can execute a weekly function that computes claim counts per user group, average discount rate differentials, and total reimbursed amounts per plan identifier. The obtained data can be converted into standardized formats suitable for downstream analytics processes. The coordinated operation between the engagement interface 220 and the reporting interface 222 can create a bidirectional communication layer within interface system 116 that allows both end-user notifications and system-level reporting functions to operate concurrently without process dependency.

System 200 can include one or more users 202, 204, and 206 that at least one (e.g., each) correspond to unique enrollment records stored within controller database 120. At least one (e.g., each) user 202, 204, and 206 can represent a network participant such as an employee, plan enrollee, or subscriber associated with at least one prescription card generated by allocation system 114. In some implementations, the users 202, 204, and 206 can provide enrollment data, such as personal identifiers or insurance references, through encrypted input channels operated by interface system 116. For example, a user can submit prescription card information through an online portal, and the controller 110 can associate the entered values with a stored mapping record linking new and original identifiers for subsequent claim translation.

The users 202, 204, and 206 can receive near real-time cost and benefit data through outbound notifications transmitted via the engagement interface 220. In some implementations, at least one (e.g., each) user can receive one or more notifications comprising cost comparisons, formulary equivalents, or available discount program offers. For example, a received notification can include a calculated co-pay value, a percentage difference between brand and generic pricing, and list references for nearby participating pharmacies offering lower reimbursement rates. The combination of user input through the interface system 116 and notification delivery through the engagement interface 220 can maintain an integrated data flow between plan enrollment operations and post-adjudication price transparency communication across controller zone 216.

The industry data source 208 can operate as an external computing system that transmits formulary data and price information to the controller 110 and the allocation system 114 for use in adjudication processes. The industry data source 208 can generate outbound data packages that include structured content describing prescription drug codes, retail pricing values, and benefit coverage tiers associated with specified therapeutic categories. In some implementations, the industry data source 208 can provide cost-related datasets at predetermined intervals such as hourly or daily refresh cycles. For example, the industry data source 208 can deliver formatted data feeds indicating wholesale acquisition costs or negotiated dispensing rates that are parsed by the controller 110 to update the plan dataset 129. The transmitted datasets can include field-level entries that correlate National Drug Code values with plan-specific copay information to facilitate precise benefit calculations during claim processing.

In some implementations, the industry data source 208 can communicate with the routing system 105 through encrypted application programming interface endpoints that implement message formats in JavaScript Object Notation (JSON) or Extensible Markup Language (XML). For example, the controller 110 can issue a periodic query through a network call to the industry data source 208 requesting updated cost structures associated with a particular prescription class. The returned data can be parsed by the allocation system 114 and written to internal data tables within the plan dataset 129 for immediate integration into claim notification workflows. The integration timing and schema parameters can be defined in rule files maintained by the controller rules dataset 124, facilitating dynamic incorporation of cost values corresponding to evolving formulary conditions.

The partner data source 210 can include one or more cooperative computing platforms programmed to provide direct data transfers between organizational networks and the prescription cost transparency platform. The partner data source 210 can transmit network plan data, enrollment metadata, or cardholder information in electronic message files interpreted by the controller 110 during mapping and association procedures. In some implementations, the partner data source 210 can be operated by an employer network that periodically sends bulk enrollment data identifying subscriber eligibility attributes. For example, a nightly batch executed by the partner data source 210 can transmit a dataset containing employee identifiers, benefit selections, and associated plan routing attributes to populate or refresh the cardholder dataset 122. At least one (e.g., each) transmitted record can be processed by the allocation system 114 to generate one-to-one linkage entries between new and original plan identifiers.

In some implementations, the partner data source 210 can operate with a scheduled synchronization sequence using secure file transfer methods such as Secure File Transfer Protocol (SFTP) or tokenized application programming interfaces. For example, the partner data source 210 can open a timed communication session through the network 130 to transmit compressed data packages in CSV or XML formats to the interface system 116. Upon receipt, the controller 110 can extract the plan identifiers and update mappings stored within the controller database 120 for subsequent translation of claim packages. The synchronization timing of the partner data source 210 can align with employer plan cycles or coverage updates to maintain consistent mapping accuracy and eliminate discrepancies between new prescription card assignments and stored original routing attributes.

The public data source 212 can act as an external database that aggregates publicly accessible pharmaceutical pricing data, manufacturer program information, or general cost benchmarks used for comparative analysis. The public data source 212 can operate on a continuous or periodic update schedule to supply open datasets identifying pharmacy discount opportunities or manufacturer-provided rebate initiatives associated with prescribed medications. In some implementations, the public data source 212 can respond to read-only request calls initiated by the interface system 116 to provide cost information reflecting region-specific retail pricing conditions. For example, when a notification event is generated by the reporting interface 222, the controller 110 can access pricing data from the public data source 212 to populate cost comparison outputs for one or more retail pharmacies in geographic proximity to a user.

In some implementations, the public data source 212 can transmit data streams formatted in JSON or XML through secure Hypertext Transfer Protocol (HTTP) connections established by the interface system 116. For example, the interface system 116 can perform an automated daily request to the public data source 212 to retrieve updated pricing and rebate tables representing retail pharmacies and discount plan programs. The allocation system 114 can analyze selected entries to calculate potential lower-cost substitution results (e.g., lower-cost alternatives), which can be merged into the plan dataset 129 for use by the reporting interface 222 when producing notifications. The synchronization of data from the public data source 212 can thereby facilitate dynamic presentation of competitive drug purchase options during real-time consumer notification events.

The processing zone 218 can include one or more network processing systems 140 that execute claim adjudication and external data aggregation operations through coordinated communication exchanges with an originator 150. At least one (e.g., each) network processing system 140 can be connected to the controller zone 216 through a bidirectional pathway provided by the network 130. The received claim packages can include a set of original bank identification numbers, processor control numbers, group identifiers, and member identifiers that have been substituted by the controller 110 during translation. In some implementations, the network processing systems 140 can process these translated claim packages in accordance with adjudication parameters maintained by benefit management engines or claims rule databases. For example, a network processing system 140 can determine a reimbursement amount, plan coverage status, or rejection code based on benefit plan data associated with the identifiers included in the translated package.

The originator 150 can transmit outbound packages through the network 130 using communication interfaces that implement segment formatting protocols such as National Council for Prescription Drug Programs (NCPDP) D.0 or SCRIPT standards. The originator 150 can generate an outbound package containing the second set of routing attributes and include additional message headers corresponding to a transaction type or prescription service code. In some implementations, the originator 150 can transmit the package to the claim editor 170 operating within the controller zone 216, which can apply translation logic to replace the new identifiers with the original identifiers prior to routing to the appropriate network processing system 140. For example, a retail pharmacy server operating as the originator 150 can send a claim for a prescribed drug containing the new group and processor identifiers, and the pre-editor 172 can substitute those fields with the original values stored in the card ID dataset 182.

When the network processing systems 140 complete adjudication, a claim response can be generated and transmitted back through the network 130 to the controller zone 216 using an asynchronous message return protocol. At least one (e.g., each) response can contain adjudicated fields such as prescription cost, pay amounts, plan code identifiers, and message segments describing claim status. In some implementations, the post-editor 174 can receive the response at the routing system 105 and append metadata identifying the network processing system 140 that adjudicated the claim. For example, a claim response can be modified by the post-editor 174 to include a network reimbursement identifier and a timestamp reflecting the adjudication event. The controller 110 can forward the modified response to the originator 150 in a format compatible with its receiving software interface, allowing the pharmacy system to parse the adjudicated result accurately.

In some implementations, the processing zone 218 can apply multi-network routing gateways that facilitate claim exchanges and response distribution between parallel adjudication networks. At least one (e.g., each) gateway can operate according to routing rules managed by the router rules dataset 184 and can select an outbound communication path based on the first set of routing attributes. For example, a gateway within the processing zone 218 can receive a translated package from the controller zone 216, match the original identifiers to a destination processing node within one of several PBM networks, and send the package using encrypted application programming interface calls. Upon receiving the corresponding adjudicated response, the gateway can deliver the package payload back across the network 130 to the interface system 116, which can transmit the response to the originator 150 for presentation to a dispensing system or user terminal operating at the point of sale.

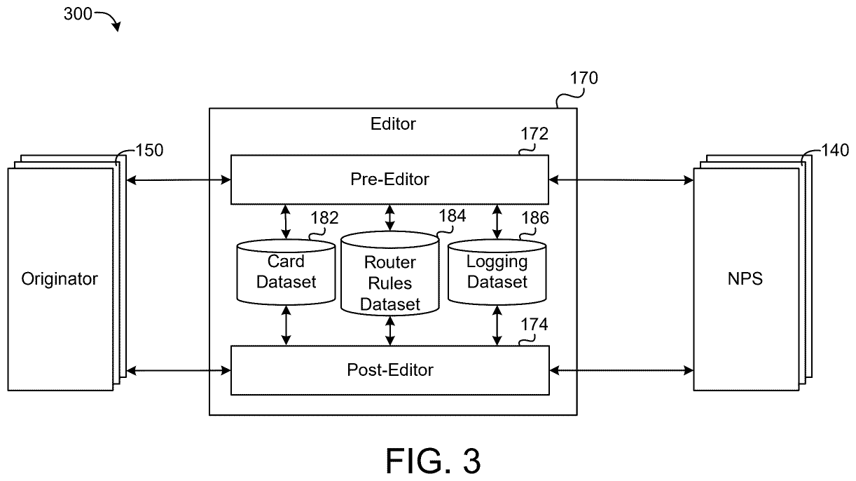

Referring now to FIG. 3, illustrated is a block diagram of an editor system 300 interfacing between an originator 150 and one or more network processing systems 140. The editor system 300 can include an editor 170 having a pre-editor 172, a post-editor 174, and associated datasets including a card ID dataset 182, a router rules dataset 184, and a logging dataset 186.

The editor 170 can operate as an intermediary system that processes claim data exchanged between an originator 150 and one or more network processing systems 140. The editor 170 can apply pre-editing and post-editing operations that modify claim message structures and field contents to maintain alignment between network origin identifiers and benefit management identifiers prior to and following adjudication. In some implementations, the editor 170 can perform pre-editing operations that identify specific message segments containing secondary routing attributes such as new bank identification numbers, processor control numbers, group identifiers, and/or member identifiers, among others. For example, the editor 170 can use the card ID dataset 182 to perform memory queries that locate the corresponding original attribute set linked to the same user record and rewrite the claim message with those original values before the message is delivered to the network processing systems 140 for adjudication. The pre-editing process can therefore generate a translated claim record that aligns with the processing system's expected routing configuration and facilitates accurate claim adjudication.

The pre-editor 172 can execute real-time translation routines that apply field-level substitution instructions maintained in the router rules dataset 184. The pre-editor 172 can parse incoming claim packets received through a network interface and determine whether one or more identifiers correspond to a secondary identifier set associated with the user. In some implementations, the pre-editor 172 can perform sequential comparisons of message headers and data elements against mapping entries retrieved from the card ID dataset 182. For example, the pre-editor 172 can inspect a claim request formatted under the National Council for Prescription Drug Programs (NCPDP) standard and replace at least one (e.g., each) new identifier field with its corresponding original identifier field based on one-to-one mapping definitions. Once the replacement is completed, the updated claim message can be generated as a temporary package containing the translated routing information that is transmitted to the appropriate network processing system 140 for further evaluation.

The post-editor 174 can perform an inverse operation that executes after a network processing system 140 adjudicates a claim and transmits a response back through the network. The post-editor 174 can receive the adjudicated response, determine the responding processing system based on identifying metadata or response structure, and modify the message to append or adjust informational elements before forwarding the response to the originator 150. In some implementations, the post-editor 174 can refer to translation data stored in the router rules dataset 184 to locate which source mapping entry corresponds to the response and insert data such as a reimbursement identifier or a network processing system identifier. For example, the post-editor 174 can generate a segmented message payload that includes updated field values describing the adjudicating network and a timestamp or message sequence element used for correlation with the original claim. The processed response can then be transmitted back to the originator 150 in a format consistent with its receiving standards.

The system 300 can store, in a network user dataset accessible by one or more processing circuits, network plan information in a standardized routing format for a plurality of users. In some implementations, the network plan information for at least one user of the plurality of users can include a first set of routing attributes expressed in the standardized routing format corresponding to a computer network processing system 140. For example, the first set of routing attributes can include an original bank identification number, a processor control number, a group identifier, and a member identifier that collectively identify a standardized network record interpretable by the computer network processing system 140 during adjudication. The network user dataset can maintain the standardized routing format across all stored records so that at least one (e.g., each) distinct user entry preserves compatibility with multiple network processing systems 140 operating in different computing environments. The standardized routing format can be defined by data structures within the card ID dataset 182 and can include field constraints, identifier schemas, and header positioning required by the editor 170 when translating claim information between the originator 150 and the network processing systems 140.

The system 300 can provide, over a computer network, remote access to the originator 150 so that the originator 150 can submit, in real time, packages containing routing information expressed in a non-standardized format dependent on a hardware and/or software platform of the originator 150. In some implementations, the routing information included in at least one (e.g., each) package can represent a second set of routing attributes that differs in schema, field order, or data-type encoding from the standardized routing format stored in the network user dataset. For example, the originator 150 can generate an electronic claim message using application software that transmits identifiers formatted according to a proprietary implementation or localized protocol syntax rather than the standardized structure required by the network processing systems 140. The editor 170 can receive these non-standardized packages through its communication interface, allowing at least one (e.g., each) inbound message to be parsed by the pre-editor 172 and associated with a corresponding record stored in the card ID dataset 182 based on the second set of routing attributes provided.

The one or more processing circuits within the editor 170 can convert the second set of routing attributes in the non-standardized format into the first set of routing attributes in the standardized routing format by replacing associated elements for at least one (e.g., each) received package. In some implementations, the pre-editor 172 can perform real-time substitution operations that identify incoming claim fields representing new identifiers and replace those values with mapped original identifiers retrieved from the card ID dataset 182. For example, when a claim request including a new bank identification number and processor control number is received from the originator 150, the pre-editor 172 can access corresponding mapping entries, apply the standardized routing format, and generate updated routing information corresponding to the appropriate computer network processing system 140. The updated routing information can be stored in the network user dataset in the standardized routing format to maintain alignment with previously standardized records. The editor 170 can then send, over the computer network and in real time, the modified package containing the updated routing information to the intended computer network processing system 140 for adjudication and further processing.

In some implementations, the combination of storing routing attribute information in network-accessible datasets, associating multiple sets of identifiers corresponding to distinct card formats, and programmatically replacing the second set with the first set in real time prior to adjudication produces a specific improvement in computer functionality. In some implementations, the routing system 105 operates to receive heterogeneous claim data expressed in formats dependent on the originator 150 hardware or software platform and consolidate those data structures into standardized routing attributes interpretable by network processing systems 140. For example, the claim editor 170 can translate user-submitted claim identifiers that differ across pharmacy systems into the original standardized identifiers before those claims rat least one (e.g., each) downstream processor environments, thereby allowing real-time, cross-network compatibility. The coordinated operations of intercepting, converting, and transmitting the translated claim packages across secured network interfaces extend beyond data manipulation in the abstract and provide a technical solution that facilitates immediate interoperability between heterogeneous claim networks and real-time visibility of prescription costs. Accordingly, the processes is a specific and practical implementation that improves the operation of network-based routing and adjudication systems themselves.

In some implementations, the editor system 300 can implement a computer network routing system that improves how distributed originator systems and multiple network processing systems exchange routing information. The one or more processing circuits can store, in a network user dataset such as the cardholder dataset 122 and/or the card ID dataset 182, network plan information for a plurality of users, where the network plan information for at least one user comprises a first set of routing attributes that defines a standardized routing format used by a corresponding network processing system 140. The same processing circuits can associate, in the network user dataset, a second set of routing attributes with the first set of routing attributes for the at least one user, where the second set of routing attributes is configured for use by the originator 150 when submitting packages and can vary based on the originator's hardware platform, software platform, or message schema. In this arrangement, the editor system 300 maintains, for at least one (e.g., each) network processing system 140, a standardized routing representation used by that network processing system 140 while permitting the originator 150 to submit routing information in a format native to the originator.

When the originator 150 transmits a package containing the second set of routing attributes associated with the at least one user, the pre-editor 172 can receive the package over the network and parse routing fields that carry the second set of routing attributes. The pre-editor 172 can then perform a conversion operation in which the second set of routing attributes, which can be non-standardized with respect to the routing configuration of the applicable network processing system 140, is replaced with the first set of routing attributes stored in the network user dataset. This replacement generates updated routing information that conforms to the standardized routing format used by the selected network processing system 140, regardless of the particular format in which the routing attributes were input by the originator 150. The conversion can be executed automatically by the one or more processing circuits whenever a package containing the second set of routing attributes is received and matched to a stored association, without requiring manual intervention or static reconfiguration of originator-side routing tables.

After the updated routing information has been generated, the interface system 116 can transmit, in real time, the package comprising the updated routing information over the network to the appropriate network processing system 140 for processing. Because at least one (e.g., each) package is converted into the standardized routing format used by the target network processing system 140 before transmission, multiple originator systems 150 using different routing attribute formats can share transaction data with a plurality of network processing systems 140 that at least one (e.g., each) operate according to their own standardized routing formats. The editor system 300 therefore allows remote originator systems to submit packages in their own routing formats while ensuring that at least one (e.g., each) network processing system 140 receives packages in a consistent, standardized routing format suitable for adjudication and other processing operations.

The combination of storing associated routing attribute sets, automatically converting originator-specific routing attributes into standardized routing attributes for the corresponding network processing systems 140, and transmitting updated packages in real time to those network processing systems 140 can provide a specific improvement over prior routing systems that relied on static mappings or required at least one (e.g., each) originator to conform to a single routing format. By integrating the routing attribute translation into the operation of the editor system 300, the disclosed techniques improve the functioning of the computer network routing system itself, allowing distributed systems to exchange routing information in real time in standardized formats defined by the network processing systems 140, regardless of the format in which the routing attributes were originally input by the originator 150.

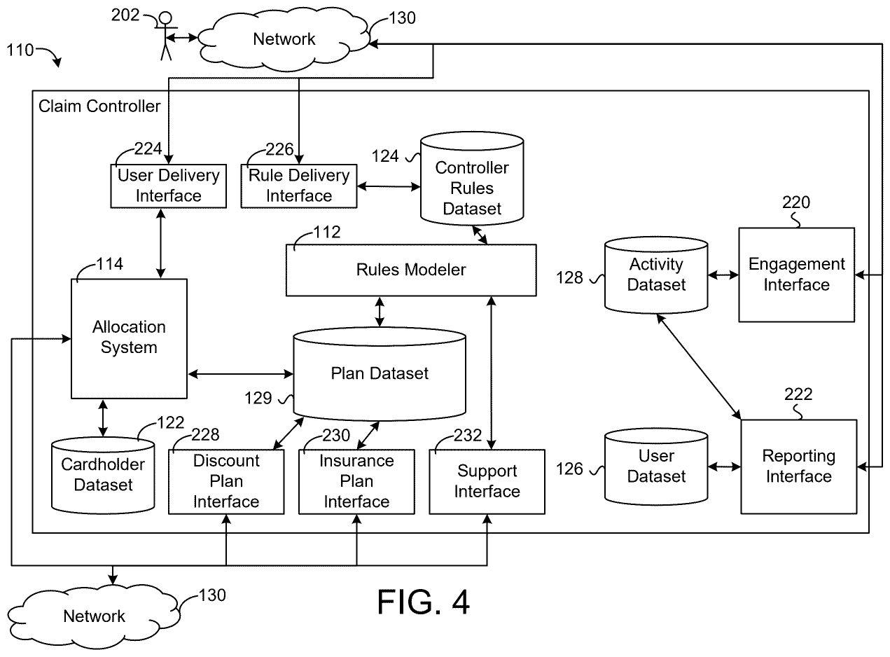

Referring now to FIG. 4, illustrated is a block diagram of a controller 110 that interfaces with associated datasets and network interfaces to execute claim routing and data management operations. The controller 110 can include a rules modeler 112, an allocation system 114, a controller database 120, and multiple network-connected interfaces such as a user delivery interface 224, a rule delivery interface 226, a discount plan interface 228, an insurance plan interface 230, a support interface 232, an engagement interface 220, and a reporting interface 222. The controller database 120 can include datasets such as a cardholder dataset 122, a controller rules dataset 124, a user dataset 126, an activity dataset 128, and a plan dataset 129. The controller 110 can also communicate with one or more networks 130 and users 202.

The user delivery interface 224 can be a communication interface that facilitates the exchange of data packets and messages between the controller 110 and one or more users 202 over the network 130. The user delivery interface 224 can use message formatting protocols compatible with the communication layer of the controller 110 to transmit structured data objects representing plan-related information or transaction results. In some implementations, the user delivery interface 224 can retrieve output data generated by the allocation system 114 or datasets 126-129 and assemble serialized message payloads for user delivery. For example, the user delivery interface 224 can acquire adjudicated prescription cost records stored in the plan dataset 129 and transmit cost transparency updates as formatted message objects containing fields such as claim identifiers, pricing components, and alternative medication options. At least one (e.g., each) message object transmitted by the user delivery interface 224 can include header and body segments referencing user identifiers and communication channel identifiers to maintain correspondence between database entries and external user endpoints, such as web interfaces or mobile devices, among others.

In some implementations, the user delivery interface 224 can manage multiple concurrent outbound communication sessions that at least one (e.g., each) represent a distinct transmission channel such as electronic mail, text message, or mobile application push message, among others. For example, the user delivery interface 224 can determine an appropriate communication mode based on a user preference indicator stored within the user dataset 126 and select a corresponding message queue for delivery. The queued messages can be serialized according to secure application programming interface schema definitions and transmitted through an authenticated connection maintained between the controller 110 and an external communication service provider. The user delivery interface 224 can monitor transmission states and, upon confirmation of successful message dispatch, can release associated buffer space for subsequent outgoing messages. At least one (e.g., each) transmission process can employ message headers containing digital certificates and encrypted payloads to maintain communication integrity when delivering real-time notifications to user endpoints.

The controller 110 can include a rule delivery interface 226 that transmits internal instruction sets, rule updates, or configuration parameters between the controller rules dataset 124 and connected operational subsystems. The rule delivery interface 226 can perform an intra-controller communication function that propagates real-time routing instructions to the allocation system 114 whenever the rules modeler 112 generates new mapping definitions. In some implementations, the rule delivery interface 226 can encapsulate at least one (e.g., each) rule update into a structured message packet that includes version identifiers, rule type indicators, and destination subsystem references. For example, when a modified mapping rule updates the relationship between a specific processor control number and a corresponding processing endpoint, the rule delivery interface 226 can transfer that update through a local bus communication sequence that refreshes the operational rules cache of the allocation system 114 during runtime processing.

In some implementations, the rule delivery interface 226 can implement an inter-process communication (IPC) protocol that facilitates data consistency across controller 110 subsystems executing in parallel process spaces. For example, the rule delivery interface 226 can encode at least one (e.g., each) update message in a JavaScript Object Notation (JSON) format or Structured Query Language (SQL) update call and broadcast it across subscribed controller components. At least one (e.g., each) receiving subsystem can parse the update message to align internal routing behavior with the latest rules data definitions stored in the controller rules dataset 124. The rule delivery interface 226 can maintain synchronization timing to ensure that message broadcasts correspond to the most recent transaction cycle, thereby preventing application delays during claim routing operations. In some implementations, the rule delivery interface 226 can use atomic update sequences to preserve transactional integrity across concurrent rule changes processed by multiple controller subsystems.

The controller 110 can include a discount plan interface 228 that operates as a data exchange conduit between the plan dataset 129 and external discount data repositories. The discount plan interface 228 can execute scheduled data retrieval procedures that query remote discount databases for pricing or rebate information relevant to prescription drug identifiers. In some implementations, the discount plan interface 228 can issue rule-based queries composed in an application programming interface schema defined by third-party discount or manufacturer data systems. For example, the discount plan interface 228 can execute a request containing a specific National Drug Code and receive in response structured data fields defining eligible rebate values, available coupon offers, or copay assistance programs. The retrieved data can be used by the controller 110 to enrich plan dataset 129 entries corresponding to the drug identifiers associated with current user prescriptions.

In some implementations, the discount plan interface 228 can periodically transmit synchronization requests using task scheduling routines that operate on predetermined intervals such as hourly or daily. The transmitted requests can include authentication tokens, timestamps, or dataset index markers that specify the range of records requiring update. For example, the discount plan interface 228 can send a scheduled refresh command to a manufacturer rebate server once every twenty-four hours to obtain the latest pricing adjustments across multiple prescription categories. The received updates can overwrite or append plan dataset 129 records with new entries so that subsequent cost calculation notifications generated by the reporting interface 222 incorporate the most current pricing information. In some implementations, the discount plan interface 228 can maintain concurrent query sessions to multiple external endpoints to achieve continuous data synchronization between the controller 110 and distinct discount or rebate sources.

The controller 110 can include an insurance plan interface 230 that performs data ingestion and exchange operations between the controller 110 and external insurer networks. The insurance plan interface 230 can receive inbound network data packages containing subscriber enrollment, coverage level, or plan modification information and parse that data for storage in the controller database 120. In some implementations, the insurance plan interface 230 can perform bidirectional communication that allows outbound delivery of processed plan records to employer portals or benefit administration systems. For example, an insurer network can provide a batch upload file containing a set of new group identifiers and processor control numbers that the insurance plan interface 230 transfers into the plan dataset 129 for incorporation into routing associations during claim translation sequences. The insurance plan interface 230 can issue acknowledgment messages confirming successful receipt and parsing of transferred records prior to initiating linkage updates between cardholder dataset 122 and plan dataset 129 entries.

In some implementations, the insurance plan interface 230 can facilitate differential synchronization routines that detect modified fields within received plan data and apply incremental updates to the controller database 120. For example, when only a subset of benefit tiers has changed in an insurer's coverage matrix, the insurance plan interface 230 can identify affected record keys and perform targeted replacements without reloading the entire data table. The insurance plan interface 230 can transport plan files via Secure File Transfer Protocol (SFTP) communications or message transfer protocol adapters to maintain compatible data formatting with external insurer infrastructures. The delivered data objects can include policy identifiers, expiration data, and coverage descriptors populating benefit tables used by network processing systems 140 during claim adjudication. The insurance plan interface 230 can maintain consistent data schema alignment so that imported plan files remain functionally compatible with mapping and substitution operations executed by the controller 110.

The controller 110 can include a support interface 232 that facilitates internal service communication between administrator consoles and operational datasets managed by the controller 110. The support interface 232 can process structured request messages to retrieve performance-related data metrics from datasets 126, 128, or 129, among others. In some implementations, the support interface 232 can aggregate operational data collected from claim routing sequences and present the aggregated results through a software console to authorized maintenance operators. For example, when a diagnostic request identifies routing throughput metrics, the support interface 232 can extract timing information from the activity dataset 128 and return a structured summary message over a secure data channel. At least one (e.g., each) response message generated by the support interface 232 can contain discrete metric values formatted for visualization in a monitoring dashboard or administrative reporting utility within the controller 110 environment.

In some implementations, the support interface 232 can establish bidirectional connections to external monitoring tools that issue programmatic queries through representational state transfer (REST) application programming interfaces. For example, a remote administrative workstation can transmit a query requesting current routing latency measurements, and the support interface 232 can respond with numerical parameters representing average and maximum processing times for recent transactions. The support interface 232 can process concurrent telemetry requests within isolation contexts that prevent interference with active routing operations managed by the allocation system 114. In some implementations, the support interface 232 can generate formatted outputs compatible with third-party compliance visualization systems, allowing routine performance inspection through authenticated viewing sessions. At least one (e.g., each) interaction through the support interface 232 can maintain the operational integrity of the controller 110 while facilitating discrete insight into data flow characteristics among its datasets and interfaces.

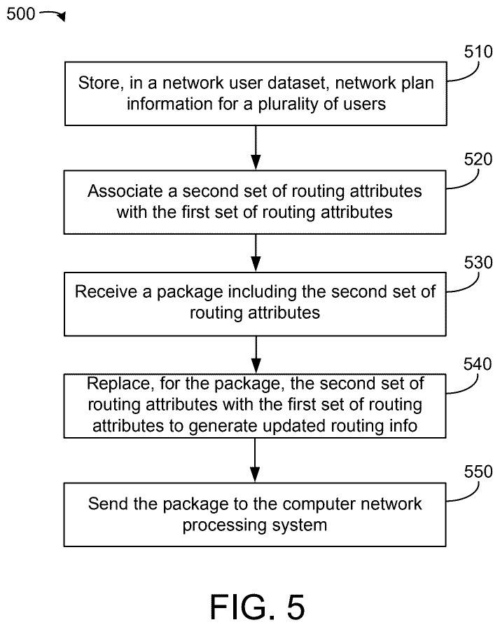

Referring now to FIG. 5, depicted is a flow chart illustrating a method 500 of updating routing information for packages based on user network plan attributes. The method 500 can be executed by any computing device of the routing system 105 that includes one or more processing circuits. The method 500 can include storing network plan information (block 510) in a network user dataset, associating routing attributes (block 520) within the stored data, receiving a package including routing attributes (block 530), replacing the routing attributes to generate updated routing information (block 540), and sending the package to a network processing system (block 550).

In some implementations, block 510 can cause the routing system 105 to persistently store network plan information for multiple users within a network user dataset. The stored network plan information can include a first set of routing attributes such as an original bank identification number, an original processor control number, an original group identifier, and an original member identifier associated with a user's insurance plan. In some implementations, the controller 110 can receive this information from an employer system or from a user device and write at least one (e.g., each) record into encrypted database tables within the controller database 120. For example, the controller 110 can assign a unique internal identifier to at least one (e.g., each) plan record and associate it with the original identifiers during initialization of a subscriber enrollment sequence prior to any claim submission, thereby defining a stored reference set of attributes to be used for subsequent routing operations.

In some implementations, block 520 can cause the controller 110 to generate a linkage between a second set of routing attributes and the first set of routing attributes associated with at least one (e.g., each) stored user record. The association can define a one-to-one mapping used for translating new prescription card identifiers into their corresponding original insurance plan identifiers. In some implementations, the allocation system 114 can generate the new identifiers programmatically, and the controller 110 can insert relational mapping entries into the controller database 120 using a mapping logic routine that establishes explicit pairings between new and original attribute values. For example, the mapping logic can generate a record that pairs a new bank identification number and processor control number with their original equivalents stored for the same user so that future replacement operations performed by the pre-editor 172 occur deterministically based on data integrity maintained between the two attribute sets.

In some implementations, block 530 can occur when the routing system 105 receives an inbound package including the second set of routing attributes from the originator 150 through the network 130. The package can represent an electronic prescription claim transmitted using National Council for Prescription Drug Programs message formatting standards and can include new identifiers associated with a recently issued prescription card. In some implementations, the interface system 116 can receive the package over a Transport Layer Security encrypted connection and transfer the message payload to the pre-editor 172 for inspection prior to translation. For example, the pre-editor 172 can determine the identity of the submitting originator 150 based on message header values and validate that the package corresponds to a user record for which both the first and second attribute sets have been previously associated in the controller database 120.

In some implementations, block 540 can cause the pre-editor 172 to substitute at least one (e.g., each) second routing attribute in the received package with the corresponding first routing attribute stored in the network user dataset. The pre-editor 172 can apply translation logic retrieved from the router rules dataset 184 to perform field-level substitution across the data segments of the prescription claim. In some implementations, the pre-editor 172 can execute in-memory lookup routines that query the card ID dataset 182 to match at least one (e.g., each) new bank identification number, processor control number, group identifier, or member identifier with its original value. For example, the pre-editor 172 can analyze a claim record and rewrite every affected routing field to generate updated routing information aligned with the configuration expected by the network processing system 140 prior to adjudication.

In some implementations, block 550 can cause the translated package containing the updated routing information to be transmitted from the routing system 105 to one or more network processing systems 140 over the network 130. The interface system 116 can transmit the updated claim package immediately after the replacement operation without intermediate storage. In some implementations, the interface system 116 can invoke network transmission routines that serialize the modified claim message into a Hypertext Transfer Protocol Secure (HTTPS) message structure suitable for submission to the external adjudication endpoint. For example, the controller 110 can identify the processing destination based on the first set of routing attributes, generate a properly addressed message header referencing that destination, and send the complete package through the network 130 to the designated pharmacy benefit manager for adjudication, thereby completing the real-time routing cycle described in the method 500.

The first set of routing attributes can include at least one of an original bank identification number (BIN), an original processor control number (PCN), an original group identifier, and an original member identifier that are stored within network plan information of at least one user. In some implementations, the second set of routing attributes can include at least one of a new BIN, a new PCN, a new group identifier, and a new member identifier that are generated for a prescription card associated with the same user. For example, a claim editor comprising a pre-editor can receive a package containing the second set of routing attributes from a computer network originator system and replace at least one (e.g., each) new identifier with its corresponding original identifier to generate updated routing information associated with a computer network processing system. The updated routing information can be produced through a BIN-over-BIN translation that substitutes every new BIN, PCN, group identifier, and member identifier field with the respective original BIN, PCN, group identifier, and member identifier derived from the network plan data of the user.

In some implementations, the operations can include receiving, from the computer network processing system, a claim response that corresponds to the package comprising the updated routing information and generating, without submitting any additional package, a notification for the user. For example, the notification can include a final cost of a prescribed drug under the user's network plan and pricing information associated with one or more lower-cost alternatives derived from adjudication results. The claim response can be modified by inserting information identifying the computer network processing system, such as by updating at least one response network segment field with a reimbursement identifier and updating at least one response message segment field with a message indicating a processing system that adjudicated the package. The operations can further include transmitting the modified claim response to the computer network originator system for display or further processing.

The computer network routing system can generate, for the user associated with the processed package, a notification containing pricing information derived from the claim response received from the computer network processing system. In some implementations, the pricing information can include a patient pay amount, pricing for one or more alternative prescription plans associated with other network processing systems, or pricing for discount plans that apply to the prescribed drug. For example, the notification can incorporate information describing at least one copay assistance program or an identified lower-cost alternative such as an on-formulary version of the drug, a generic equivalent, or availability of the same medication at a different originator system offering a more favorable price. The notification can be transmitted to a user device through one or more communication channels such as a text message, an email message, or a mobile application message in real time following receipt of the adjudicated response.

In some implementations, the operations can further include obtaining, from one or more data sources, plan data such as discount plan data and/or network plan data and storing the plan data in a plan dataset to determine the pricing information and the lower-cost alternative reported to the user. The process of storing network plan information for multiple users can involve writing, in a network user dataset, a first set of routing attributes that represent at least one (e.g., each) user's original routing identifiers and establishing a mapping between the first set of routing attributes and the second set of routing attributes that comprise new routing identifiers. For example, the one or more processing circuits can intercept a package containing the second set of routing attributes from the computer network originator system before transmission to a processing system, apply the mapping stored in the network user dataset, and generate updated routing information corresponding to the intended processing destination based on the associated first set of routing attributes.

The routing system can transmit, in real time, the package that includes the updated routing information to a computer network processing system determined according to the associated first set of routing attributes stored for the user. In some implementations, data transmissions related to the package and the network plan information can be encrypted in transit, and the network user dataset maintaining the stored attributes can be encrypted at rest. The operations can also include receiving the first set of routing attributes from an employer system or a user device and generating a corresponding prescription card for at least one (e.g., each) user that contains the second set of routing attributes to be used by the computer network originator system when sending future claim packages.

Additionally, the present disclosure pertains to systems and methods for routing prescription drug claims between a pharmacy and a plurality of pharmacy benefit managers (PBMs). In some implementations, prescription drug claim routing can be performed on submitted drug claims from pharmacies. The prescription drug claim can include routing information that causes the pharmacy to send the prescription drug claim to the prescription drug claim routing system. The routing system can obtain a set of rules, select a PBM from a plurality of PBM to send the prescription drug claim, and modify the routing information to send the prescription drug claim to the selected PBM. Thus, the systems and methods described here describe a system to route prescription drug claims based on generated rule sets to increase the utilization of optimal pricing across PBMs and improve prescription drug claim adjudication architectures.

In general, PBMs handle prescription benefits for individuals who receive a prescription from a pharmacy. In particular, PBMs can be third-parties between individuals and/or plan sponsors (e.g., businesses, industries, schools, insurers, and governments) (collectively referred to herein as “customers”) and the pharmacies. For example, customers can pay premiums to PBMs, and in return, the PBMs can negotiate, on the customer's behalf, with the pharmacy to lower the out-of-pocket price a customer pays for a prescription drug. PBMs can also work with drug companies and insurance companies to establish drug formularies. Drug formularies can be a list of drugs (e.g., generic, brand-name, etc.) covered by the customer's health plan. In some implementations, the drug formularies can be tiered based on various categories (e.g., generic, lowest copays, non-preferred generics, brand-name medications, specialty drugs, etc.) and the tiers can be indicative of how much a customer pays out-of-pocket for a particular drug. For example, Tier 1 of a drug formulary can include Brand A drug, and Brand B drug, which are preferred based on a PBMs drug formulary and can be the lowest copay for the customer. In this example, Tier 2 of the drug formulary can include Brand C drug which is non-preferred based on the PBMs drug formulary and can cost more than a drug in Tier 1 of the drug formulary. However, at least one (e.g., each) PBM can establish a unique drug formulary such that drug placement on the unique drug formulary can be tiered differently. The unique drug formularies can be based on various factors, for example, but not limited to, safety, efficacy, cost of the drug, drug company rebates, drug company coupons, discount cards, effectiveness, if a generic version exists, etc.

In some systems, a pharmacy can submit a prescription drug claim to a PBM, and the prescription drug claim can be processed by the PBM. In some implementations, at least one (e.g., each) PBM can have multiple different plans associated with the PBM. During the processing of a prescription drug claim, the pharmacy can receive payment information including, but not limited to, what the customer must pay to receive the prescription and how much the pharmacy will receive in return for the sale. The payment information can be determined based on the prescription plan of the customer, the pharmacy's contract with the PBM, the formulary, rebates, discounts, and/or various other factors. In one example, the customer can be required to pay a copay (e.g., $10), which can be a smaller amount of money compared to the drugs typical listed price (e.g., $250). In another example, the customer can be required to pay a percentage of the drug list price (e.g., 40%), which is still likely to be a smaller amount of money compared to the drug's typical listed price. In yet another example, the customer can have not met the deductible of their prescription plan and can be required to pay the full drug list price. However, the payment information sent by PBM X to pharmacy A, can be different than what is sent to pharmacy B. This is the case because pharmacy A can have a different contract with PBM A then pharmacy B does.

Furthermore, there are a plurality of PBMs (e.g., PBM X, PBM Y, PBM Z) that can all have different contracts with different pharmacies. For example, Pharmacy A can have a contract with PBM X and PBM Y, and Pharmacy B can have a contract with PBM X and PBM Z. In this example, one customer (e.g., John Doe) can have prescription plan K with PBM X, whereas another customer (e.g., Jane Doe) can have prescription plan L with PBM Z. Thus, in this example, if Jane Doe goes to Pharmacy A and provides prescription plan L, she can have to pay out-of-pocket the full list price for her prescription because PBM Z does not have a contract with Pharmacy A. If John Doe goes to Pharmacy A and provides prescription plan K, Pharmacy A would submit prescription drug claim to PBM X, and receive payment information including how much John Doe would have to pay out-of-pocket. As shown, if Jane Doe utilized PBM X, instead of PBM Z (determined by her prescription plan L), she would have reduced her out-of-pocket expenses. Additionally, Jane Doe can have been eligible for prescription plan L but was unaware. Moreover, Jane Doe and John Doe can have been eligible for prescription plan J, which could have provided the lowest out-of-pocket expenses for their prescriptions, but both were unaware of that plan. Thus, the ability to route prescription drug claims, such that a drug claim system can select an eligible PBM from a plurality of eligible PBMs to process a prescription drug claim, provides customers with lower out-of-pocket expenses.

In order to address this problem, the present disclosure describes a system that allows pharmacies to submit prescription drug claims to a prescription drug claim routing system instead of a particular PBM to enhance payment plan flexibility for customers (e.g., reducing cost, allowing pharmacy flexibility, reducing complexity of purchasing drugs, etc.). The prescription drug claim routing system can utilize a customizable selection criteria to route submitted prescription drug claims to appropriate PBMs for processing before the payment information is provided back to the pharmacy. In some implementations, the customer can present a single prescription card to the pharmacy but can be associated with a plurality of prescription plans across a plurality of PBMs. Therefore, aspects of the present disclosure address problems in handling prescription drug claims by routing particular claims to particular PBMs utilizing a drug claim processing architecture with a customized PBM selection criteria based on the customer.

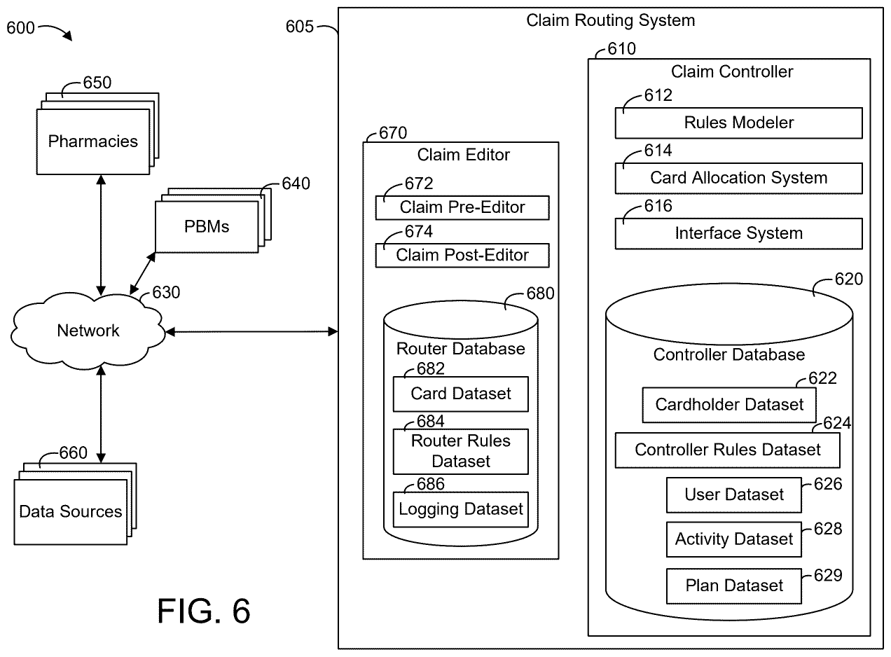

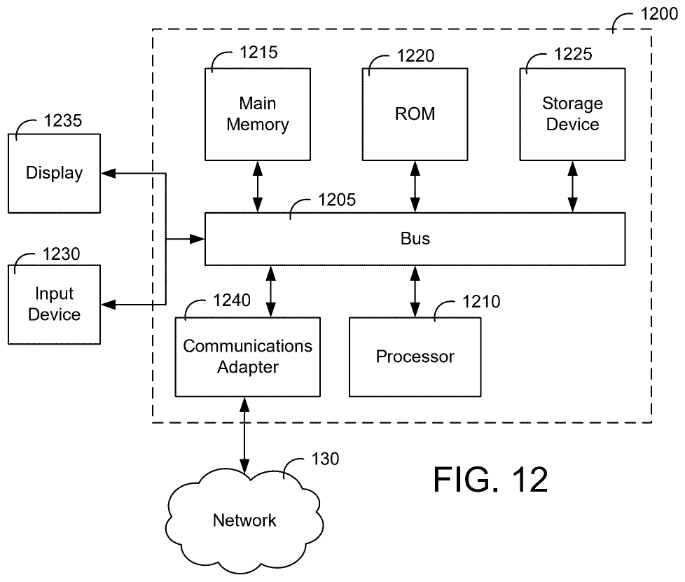

Referring now to FIG. 6, a block diagram of a claim routing system 605, and associated environment 600 is shown, according to an illustrative implementation. Network 630 can include a local area network (LAN), wide area network (WAN), a telephone network, such as the Public Switched Telephone Network (PSTN), a wireless link, an intranet, the Internet, or combinations thereof. At least one (e.g., each) system and/or database can communicate via network 630 with one or more systems in associated environment 600. The claim controller 610 can also include at least one data processing system or processing circuit, such as those described below in detail with reference to FIG. 12. The one or more processing circuits can include a microprocessor, an application specific integrated circuit (ASIC), a field-programmable gate array (FPGA), etc., or combinations thereof. A memory can include, but is not limited to, electronic, optical, magnetic, or any other storage or transmission device capable of providing processor with program instructions. Instructions can include code from any suitable computer programming language.

Claim controller 610 can include the controller database 620 which can be configured to store a variety of information relevant to processing drug claims. Information can be received by one or more PBMs 640, one or more pharmacies 650, data sources 660, and/or claim editor 670, for example. The claim controller 610 can be configured to query the controller database 620 for information and store information in the controller database 620. In various implementations, the controller database 620 includes various transitory and/or non-transitory storage mediums. The storage mediums can include but are not limited to magnetic storage, optical storage, flash storage, RAM, etc. The controller database 620 and/or the claim controller 610 can use various APIs to perform database functions (i.e., managing data stored in the geographic experiment database 620). The APIs can be but are not limited to SQL, NoSQL, NewSQL, ODBC, JDBC, etc.

Claim editor 670 can be configured to exchange data with pharmacy 650, PBMs 640, and claim controller 610. Claim editor 670 is shown to include a claim pre-editor 672 and a claim post-editor 674. The claim editor 670 can include at least one data processing system or processing circuit, such as those described below in detail with reference to FIG. 12.

Claim editor 670 can include the editor database 680 which can be configured to store a variety of information relevant to processing drug claims. Information can be received by the PBMs 640, the pharmacies 650, the data sources 660, and/or the claim controller 610, for example. The claim editor 670 can be configured to query the router database 680 for information and store information in the router database 680. In various implementations, the router database 680 includes various transitory and/or non-transitory storage mediums. The storage mediums can include but are not limited to magnetic storage, optical storage, flash storage, RAM, etc. The router database 680 and/or the claim editor 670 can use various APIs to perform database functions (i.e., managing data stored in the geographic experiment database 620). The APIs can be but are not limited to SQL, NoSQL, NewSQL, ODBC, JDBC, etc.

In some implementations, the claim controller 610 and the claim editor 670 can be implemented as separate systems or integrated within a single system (e.g., the claim controller 610 can be configured to incorporate some or all of the functions/capabilities of the claim editor 670). In various implementations, the controller database 620 and the router database 680 can be implemented as separate databases or integrated within a single database (e.g., the controller database 620 can be configured store some or all of the data of router database 680).

Referring to the claim routing system 105 generally. The claim editor 670 can receive drug claim submissions from pharmacy 650 associated with one or more requested drugs (e.g., prescription drug, doctor prescribed treatment). At least one (e.g., each) drug claim submission can have a plurality of fields. The claim editor 670 can identify the submitting pharmacy identifier (e.g., a National Board of Pharmacy (NABP) number, National Provider Identifier (NPI), etc.) and drug identifier (e.g., National Drug Code (NDC), Generic Product Identifier (GPI), etc.) from the plurality of fields in the drug claim submission. In various implementations, the drug claim submission can also be edited (e.g., in real-time) by the claim pre-editor 672 based on generated rules by the claim controller 610 and stored in router rules dataset 684. In some implementations, the generated rules can be utilized by the claim editor 670 to edit the drug claim submission such that the routing information for a target PBM can be added to the drug claim submission. The drug claim submission can further be logged in the logging dataset 686 by the claim editor 670. In some implementations, the claim editor 670 can send the edited drug claim submission to the one or more PBMs 640 based on the routing information. The one or more PBMs 640 can provide a response to claim editor 670 adjudicating (e.g., paying or denying) the edited drug claim submission after comparing the edited drug claim submission to the benefit or coverage requirements. Adjudication of the edited drug claim can include determining the level of reimbursement to the pharmacy 650 that made the drug claim submission. In some implementations, the adjudicated drug claim submission can be further edited by the claim post-editor 674 based on generated rules by the claim controller 610 and stored in router rules dataset 684. The adjudicated drug claim submission can further be logged in the logging dataset 686 by the claim editor 670. In various implementations, the claim editor 670 can send the adjudicated drug claim submission back to pharmacy 650 including the price for the one or more requested drugs.

Pharmacy 650 can be configured to exchange data including drug claim submissions with claim editor 670, over network 630. The pharmacy 650 can be any entity or system that submits a prescription claim electronically (e.g., storefront, mail order pharmacies). In general, there can be a plurality of pharmacies, at least one (e.g., each) having different contracts with different PBMs (e.g., PBM 640). For example, a customer can be prescribed a drug by their doctor and visit a pharmacy (e.g., in-person, virtually via the internet). In the following example, the customer can present their universal prescription card (UPC) to the pharmacy in which the pharmacist will initiate a drug claim submission with claim editor 670. In some implementations, a drug claim submission can include identifying information (or attributes) associated with a UPC. For example, the UPC can include, but is not limited to, an ID number (e.g., 408002), a group identifier (e.g., UNITY), a bank identification number (BIN) (e.g., 111111), and a processor control number (PCN) (e.g., AAA). In another example, the UPC can include, but is not limited to, a member name (e.g., Member A), a group identifier (e.g., 1234567), an Rx Group identifier (e.g., ABCDEF), a BIN (e.g., 222222), a medical network (e.g., Texas Plus 1), and copay information (e.g., emergency room co-pay: $75, retail Rx: $10/$25/$45, mail-order Rx: $25/$52/$112). The data can be data inputted for particular entities or users (e.g., customers, customer purchases) at one or more points in time at pharmacy 650.

PBMs 640 can be configured to exchange data including drug information with claim editor 670 over network 630. The PBM 640 can be any entity or system that adjudicates (or processes) prescription drug claims. In general, there can be a plurality of PBMs, at least one (e.g., each) having different contracts with different pharmacies (e.g., pharmacies 650). For example, one PBM of PBMs 640 can provide discount drug pricing to be used in comparing plans across multiple PBMs. In another example, another PBM of PBMs 640 can provide both discount drug pricing, as well as insured copay pricing for registered plans and members to be used in comparing plans across multiple PBMs. In yet another example, yet another PBM of PBMs 640 can provide copay benefit information for registered plans and members to be used in comparing plans across multiple PBMs. In various implementations, claim editor 670 can provide an edited drug claim submission to the one or more PBMs 640. Upon receiving an edited drug claim submission, the PBMs 640 can adjudicate the drug claim and provide a response including the adjudicated drug claim submission and the level of reimbursement to pharmacy 650 (e.g., via network 630 and claim routing system 105). In some implementations, at least one (e.g., each) PBM can have multiple different plans associated with the PBM.

Data sources 660 can include data collected by the claim controller 610 based on receiving discount plan data and insurance plan data from various customers (e.g., businesses, industries, schools, insurers, partners, public entities, PBMs, and governments) via network 630. The data can include identifying information associated with a discount plan and/or insurance plan. For example, the discount plan data could include a plurality of discounts plans collected from various customers. In another example, the insurance plan data could include a plurality of insurance plans collected from various customers. The discount plan data and/or insurance plan data can include data associated with a plurality of entities, a plurality of users, a specific entity, a specific user, etc. Data sources 660 can also be various data aggregating systems and/or entities that collect discount plan data and/or insurance plan data. This information can be stored as plan data in the plan dataset 629.

Claim controller 610 can include one or more systems (i.e., computer-readable instructions executable by a processor) and/or circuits (i.e., ASICs, Processor Memory combinations, logic circuits, etc.) configured to perform various functions of the claim controller 610. In some implementations, the systems can be or include a rules modeler 612, a card allocation system 614, and an interface system 616. It should be understood that various implementations can include more, fewer, or different systems than illustrated in FIG. 6, and all such modifications are contemplated within the scope of the present disclosure.

Referring to the claim controller 610 generally. The rules modeler 612 can be configured to collect plan information from discount plans and insurance plans (e.g., from data sources 660) and store the collected plan information into the plan dataset 629. The card allocation system 614 can be configured to generate cards for one or more individuals and/or customers and store card data in cardholder dataset 622. The interfaces system 616 can be configured to generate and provide one or more interfaces, such as but not limited to, discount plan interface, insurance plan interface, support interface, engagement interface, reporting interface, user delivery interface, and rule delivery interface. One or more interfaces of the interface system 616 can be configured to communicate (e.g., query, store) with one or more datasets of controller database 620 (e.g., user dataset 626, activity dataset 628, and plan dataset 629).

Claim editor 670 can include one or more systems (i.e., computer-readable instructions executable by a processor) and/or circuits (i.e., ASICs, Processor Memory combinations, logic circuits, etc.) configured to perform various functions of the claim editor 670. In some implementations, the systems can be or include a claim pre-editor 672 and a claim post-editor 674. It should be understood that various implementations can include more, fewer, or different systems than illustrated in FIG. 6, and all such modifications are contemplated within the scope of the present disclosure.

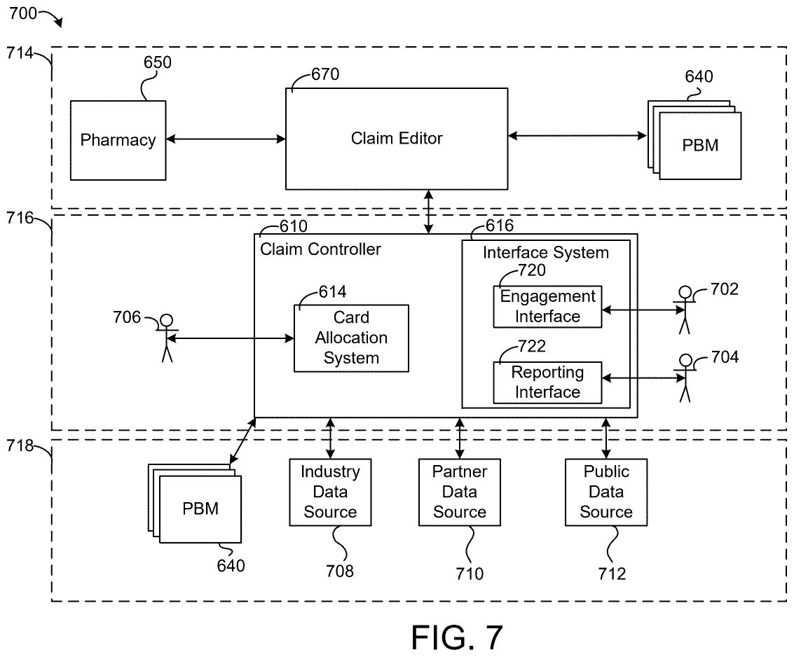

Claim pre-editor 672 can be configured to modify received drug claim submissions from pharmacy 650 based on one or more rules determined by rules modeler 612. The claim post-editor 674 can be configured to modify received adjudicated drug claim submissions from PBM 640 based on one or more rules determined by rules modeler 612. Additional details relating to the functions of the claim controller 610 and claim editor 670 are provided herein with respect to FIGS. 7-9.

Referring now to FIG. 7, a block diagram depicting an implementation of a claim processing architecture 700, according to an illustrative implementation. The claim processing architecture 700 is shown to include a claim processing layer 714, rules/interface (RI) layer 716, and data source layer 718, at least one (e.g., each) including various systems. As shown, data can flow bidirectionally (e.g., sent and received) from the various systems.

In some implementations, the claim processing layer 714 can process drug submission claims initiated at pharmacy 650. For example, pharmacy 650 can submit a prescription drug claim to claim editor 670 associated with the universal prescription card (UPC) of a customer. In this example, in real-time, the claim editor 670 can utilize a customizable selection criteria to route submitted prescription drug claims to one or more PBMs 640 for processing before the payment information is provided back to the pharmacy. In various implementations, the customizable selection criteria can be set by the user and/or the pharmacy. In some implementations, the customizable selection criteria can also be set by claim controller 610 based on data collected from various systems described herein (e.g., pharmacy 650, PBM 640, data sources 660). Customizable selection criteria can include, but is not limited to, lowest price for the customer, highest reimbursement for the pharmacy, highest rebate for the plan administrator, available copay and customer assistance plans based on a card user's profile, preferred or non-preferred PBM(s) based on pharmacy preferences PBMs with available generic effective rate (GER) (e.g., GER load balancing), insurance price versus cash price threshold, and/or default plan when no other customizable selection criteria are met. Additionally with reference to the above example, PBMs 640 can adjudicate the prescription drug claim and route the payment information back to the claim editor 670. As shown, the customer and pharmacy can provide customizable selection criteria but the claim editor 670 can determine which PBM of the PBMs 640 the prescription drug claim gets routed to. In some implementations, the customer and/or pharmacy can indicate a specific PBM (or a plurality of PBMs) the prescription drug claim should be routed to. In some implementations, all communicate between layers of the one or more processing circuits can be done over network 630 (resembling similar features and functionalities of network 630 in FIG. 6). Furthermore, in various implementations, at least one (e.g., each) PBM can have multiple different plans associated with the PBM and the selection process can in selecting not only a PBM, but also a plan provided by that PBM. For example, a PBM can provide prescription plan A and prescription plan B. In this example, the claim editor 670 can select prescription plan A, prescription plan B, or both of them based on the customizable selection criteria. Further in this example, if both prescriptions plans are selected, the claim editor 670 can route the prescription drug claim to the same PBM but with different routing information based on the prescription plan.

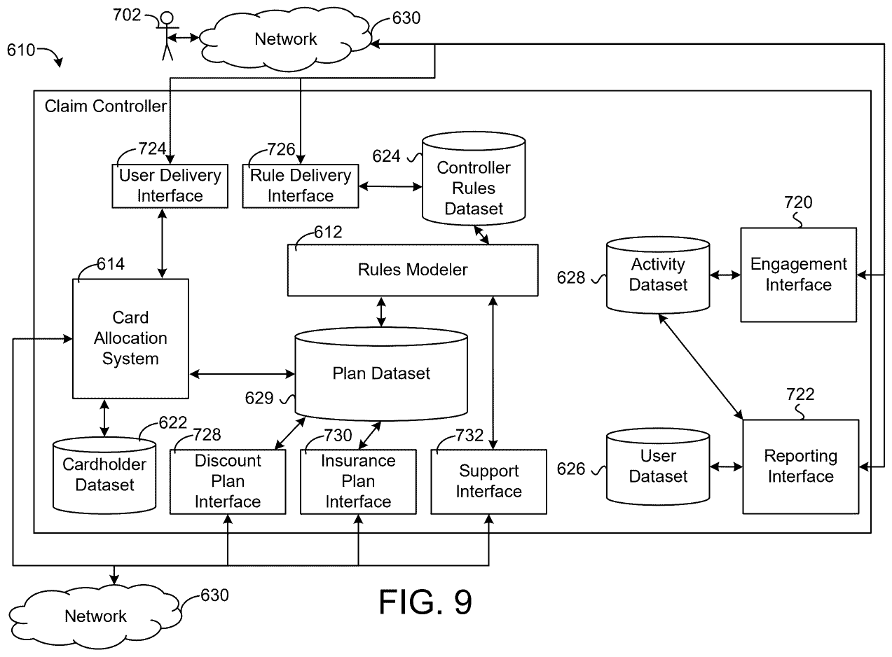

In some implementations, the RI layer 716 can receive requests from one or more processing circuits of claim editor 670. For example, claim controller 610 can send (e.g., continuously, autonomously, on request) claim processing rule structures (or dataset) to claim editor 670. In various implementations, the RI layer 716 can receive communication at the card allocation system from user 706. For example, card allocation system 614 can receive a new UPC request. In this example, the card allocation system 614 can generate a unique UPC for user 706 including various information such as, but not limited to, a bank identification number (BIN) (e.g., 222222), a processor control number (PCN) (e.g., AAA), a group identifier (ID) (e.g., UNITY), and a cardholder identifier (e.g., 123321jd). In some implementations, the RI layer 716 can also receive customer communication at the interface system 616, in particular, engagement interface 720 and reporting interface 722 from user 702 and user 704. In some implementations, user 702, user 704, and user 706 can be a computing device (e.g., user device, smart device, mobile device, computer, etc.). In some implementations, the engagement interface 720 can be configured to communicate (e.g., via network 630) with user 702 based on event triggers. An event trigger can be a configuration set by a user (e.g., 702, 704) such that at least one (e.g., each) user can receive customized notifications based on the event triggers. For example, a message (e.g., text, email, application notification) can be sent to user 702 (e.g., customer), over network 630, notifying user 702 a refill can be needed for a previous prescribed drug. In this example, user 702 can have set an event trigger such that a notification is provided when a refill can be needed. In another example, a message can be sent to user 702 (e.g., prescriber), over network 630, notifying user 702 of real-time any outstanding medications that have not been picked up by customers. In this example, user 702 can have set an event trigger such that a notification is provided every day indicating outstanding medications for pick-up. In various implementations, reporting interface 722 can be configured to provide (e.g., via network 630) with user 704 (e.g., can be the same user as user 702, or can be a different user) data visualization tools. The one or more processing circuits of the interface system 616 can generate reports depicting activity, performance, pricing, and rules information associated with claim controller 610. For example, user 704 (e.g., pharmacist) can run a report, utilizing a graphical user interface (GUI) provided by the reporting interface 722, to analyze a customer's prescription history. In another example, user 704 (e.g., insurance company) can run a report for all its members using the UPC to calculate one or more member's remaining deductibles. In various implementations, the interface system 616 can include additional interfaces as described in detail with reference to FIG. 9.

In some implementations, the data source layer 718 can resemble similar features and functionalities of data source 660 in FIG. 6. As shown, the data sources can include an industry data source 208, a partner data source 710, and public data source 712. In various implementations, PBM 640 can also be configured to provide data as a data source. For illustration reasons, PBM 640 is shown in both the claim processing layer 714 and data source layer 718. At least one (e.g., each) data source can provide various data. For example, industry data source 208 could be a data analytics company that can provide (e.g., when queried, autonomously, scheduled) pharmaceutical data (e.g., market trends, pricing trends, new drugs, recalls, side effects, efficacy of different drugs, etc.). In another example, partner data source 710 could an insurance company that provides (e.g., when queried, autonomously, scheduled) plans for the UPC's described herein, and can provide insurance data (e.g., drug prices, co-pays, reimbursement information, plan information, etc.). In yet another example, the public data source 712 can be provide (e.g., when queried, autonomously, scheduled) public available pharmaceutical data (e.g., market trends, pricing trends, new drugs, recalls, side effects, efficacy of different drugs, Better Business Bureau (BBB) ratings for PBMs or Pharmacies, litigation information, etc.). In yet another example, at least one (e.g., each) PBM 640 can provide (e.g., when queried, autonomously, scheduled) various prescription drug information (e.g., formularies, rebates, discounts, coupons, etc.)

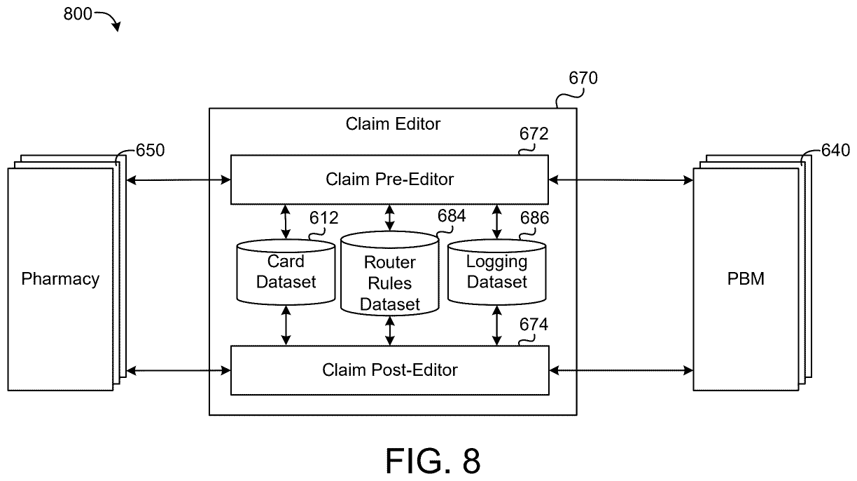

Referring now to FIG. 8, a block diagram depicting a claim editor 670 and associated environment 800, according to an illustrative implementation. As described in detail about with reference to FIG. 1, claim editor 670 can be configured to exchange data with pharmacy 650, PBM 640, and claim controller 610. Claim editor 670 is shown to include a claim pre-editor 672, a claim post-editor 674, a card dataset 682, a router rules dataset 684, and a logging dataset 686. Referring to the claim editor 670 and associated environment 800 generally. The PBMs 640, pharmacies 650, and claim editor 670 can all communicate over network 630 described in detail with reference to FIG. 6. The PBMs 640, pharmacies 650, and claim editor 670 can at least one (e.g., each) include at least one data processing system or processing circuit, such as those described below in detail with reference to FIG. 12. The pharmacies 650 can submit prescription drug claims to claim editor 670 and receive claim response with adjudicated PBM information from claim editor 670. The claim editor 670 can receive prescription drug claims from pharmacies 650 and route the prescription drug claim to one or more PBMs of PBMs 640 based on a set of rules. The claim editor 670 can also receive adjudicated prescription drug claims from the one or more PBMs and provide a claim response to pharmacies 650. In some implementations, the PBMs 640 can provide (e.g., in real-time, schedule, autonomously) pricing information (e.g., formulary, discounts, coupons, etc.) to claim editor 670. The PBMs 640 can receive routed prescription drug claims from claim editor 670 and send adjudicated prescription drug claims to claim editor 670. As shown in the architecture, the claim editor 670 can be architected as a system in between the pharmacies 650 and PBMs 640. The architecture provides improvements to prescription drug claim adjudication architectures by routing prescription drug claims based on applying a set of rules of one or more different prescription plans to one or more PBMs from a plurality of PBMs 640. This approach allows prescription drug claim adjudication architectures to provide significant improvements to the PBM submission process of prescription drug claims on a communication network and can provide significant cost savings by routing prescription drug claims to an optimal PBM based on a set of rules. The PBMs 640 and pharmacies 650 are described above in detail with reference to FIG. 1.

Referring to the claim editor 670 in more detail. The claim editor 670 can be configured to perform a claim routing process that can be triggered from incoming prescription drug claims from pharmacy 650. The prescription drug claim can include various information associated with a UPC and/or payment card. The various information can be grouped into various segments of data (collectively referred to herein as “attributes” and/or “fields”). For example, a prescription drug claim can include transaction segment fields that can include, but is not limited to, the BIN and PCN. In this example, the prescription drug claim can also include insurance segment fields that can include, but is not limited to, the Rx Group identifier, and ID number. In yet another example, the prescription drug claim can also include response insurance segment fields that can include, but is not limited to, Network Reimbursement Identifier (NRI). In yet another example, the prescription drug claim can also include response message segment fields that can include, but is not limited to, message field. The claim editor 670 can query against the router rules dataset 684 to determine the rule set (sometimes referred to herein as “a set of rules”). In various implementations, the rule set can define a set of available prescription plan options for processing the prescription drug claim, and where at least one (e.g., each) of the available prescription plan options can correspond to a PBM of the plurality of PBMs 640. In some implementations, the claim editor 670 can inquire with the PBM for pricing information (e.g., formulary, discounts, coupons, etc.). The rule set can be generated and updated by the claim controller 610. In various implementations, the prescription drug claim can also include, but is not limited to, customer segment fields (e.g., customer identifying information), prescriber segment field (e.g., prescriber identifying information), pharmacy segment fields (e.g., customer identifying information), pricing segment fields (e.g., pricing information such as, deductible, copay, etc.), or any combination.

In some implementations, the claim editor 670 can further be configured to select the PBM from the plurality of PBMs 640 to route the prescription drug claim to for adjudication. In some implementations, the claim pre-editor 672 can update the attributes of the prescription drug claim to reflect the plan and PBM that will be adjudicating (or processing) the prescription drug claim. For example, after determining the prescription drug claim should be routed to PBM A, the claim pre-editor 672 can modify the BIN (e.g., 612314), PCN (e.g., 612), Rx Group Identifier (e.g., RX07), and NRI (e.g., 524-FF) to reflect the criteria (e.g., plan scheme) for submitted the prescription drug claim to PBM A. In another example, after determining the prescription drug claim should be routed to PBM B, the claim pre-editor 672 can modify the BIN (e.g., 501203), PCN (e.g., 928), Rx Group Identifier (e.g., MPx87), and NRI (e.g., 23G-5N) to reflect the criteria for submitting the prescription drug claim to PBM B. In some implementations, the claim pre-editor can modify the received prescription drug claim multiple times and send it to multiple PBMs (e.g., PBM A and PBM B) for adjudication. In particular, in various scenarios, upon determining multiple available plan options, the claim pre-editor 672 can simultaneously send a modified prescription drug claim unique to at least one (e.g., each) of the PBMs for adjudication.

In various implementations, the claim post-editor 674 can receive adjudicated prescription drug claims, via network 630. In some implementations, if there are multiple adjudicated prescription drug claim responses the claim post-editor 674 can compare the responses and select the optimal plan. In selecting the optimal plan, the claim post-editor 674 can utilize the rule set stored in router rules dataset 684. In particular, the rule set can include customizable selection criteria set by a user or entity (e.g., customer, pharmacy, insurance provider, etc.). For example, the customizable selection criteria could be utilized by the claim post-editor 674 to select the optimal plan based on the lowest price for the customer (e.g., customized by the customer). In another example, the customizable selection criteria could be utilized by the claim post-editor 674 to select the optimal plan based on the highest reimbursement for the pharmacy (e.g., customized by the pharmacy). In yet another example, the customizable selection criteria could be utilized by the claim post-editor 674 to select the optimal plan based on the highest rebate for the plan administrator (e.g., customized by the insurance company). In yet another example, the customizable selection criteria could be utilized by the claim post-editor 674 to select the optimal plan based on the available copay and customer assistance plans (e.g., customized by the claim editor 670 based on querying the card dataset 682 and/or data sources 660). In some implementations, the claim post-editor 674 can modify the adjudicated prescription drug claim based on the PBM response and the adjudication information. In various implementations, the claim post-editor 674 can modify the NRI of the segments with PBM and/or adjudication information. For example, the claim post-editor 674 can modify the NRI of the response insurance segment (e.g., defined by the claim editor 670 and can identify the network, for the covered customer, used in calculating the reimbursement to the pharmacy), the message field of the response message segment (e.g., “Complete”), and the response pricing segment (e.g., patient pay amount, ingredient cost paid, dispending fee paid).

In various implementations, all prescription drug claims routed by the claim editor 670 (e.g., received from pharmacy 650, received from PBM 640) can be logged in logging dataset 686. In some implementations, the card dataset 682 can be queried by the claim pre-editor 672 and/or claim post-editor 674 to include card data for a plurality of UPCs and other prescription cards. In particular, the card dataset 682 can include card information and rule preferences for routing claims by claim editor 670. That is, the cardholder dataset 622 can store, but not limited to, user profile information (e.g., names, age, income, etc.), the user's UPC, insurance information (e.g., BIN, PCN, Group, ID), rule preference(s) (e.g., pick the lowest patient paid amount), plan preferences (e.g., only use X, Y, Z plans), and communication and data access opt-ins, and so on. However, the card dataset 682 can cache the card information and rule preferences associated with the cardholder dataset 622 but can be used in routing claims by claim editor 670. Further, by using the card dataset 682, this technical solution can provide improved claim processing speed of drug claims without receiving or transmitting protected or private information via the network. This not only protects claim editor 670 from exposing the protected claim and/or entity information, but also protects PBMs 640 and Pharmacies 650 from exposing their protected or private information, which is a significant improvement to the security of networking systems. Thus, the information in the card dataset 682 is not susceptible to data brat least one (e.g., each)es or man-in-the-middle attacks, which is an improvement over pharmacy submitting claims directly to PBMs. Additional details relating the issuance and allocation of cards are described in detail herein with reference to card allocation system 614.

Referring now to FIG. 9, a block diagram depicting a claim controller 610, according to an illustrative implementation. As described in detail about with reference to FIG. 1, claim controller 610 can be configured to exchange (e.g., customers, insurers, prescribers, pharmacists, caregivers) user data with one or more systems described herein, via network 630. Claim controller 610 is shown to include a rules modeler 612, a card allocation system 614, a cardholder dataset 622, a controller rules dataset 624, a user dataset 626, an activity dataset 628, a plan dataset 629, and a plurality of interfaces including, but not limited to, an engagement interface 720, a reporting interface 722, a user delivery interface 724, a rules delivery interface 726, a discount plan interface 728, an insurance plan interface 730, and a support interface 732. In some implementations, the plurality of interfaces can be implemented as separate systems or integrated within a single system (e.g., interface system 616 in FIG. 6 can be configured incorporate some or all of the functions/capabilities of some or all of the plurality of interfaces). In various implementations, the dataset (e.g., 622, 624, 626, 628, 629) can be implemented as separate databases or integrated within a single database (e.g., controller database 620 can be configured store some or all of the dataset). The claim controller 610 can include at least one data processing system or processing circuit, such as those described below in detail with reference to FIG. 12.

Rules modeler 612 can be configured to generate claim processing rule sets (collectively referred to herein as “rule sets” or “a set of rules”). The rules modeler 612 can communicate with plan dataset 629 (e.g., via querying) and support interface 732 to utilize various information to generate rule sets. In some implementations, a rule set can include a plurality of rules associated with specific fields of a prescription drug claim (described above). Profile information about a cardholder such as personal information collected by the card allocation system 614 and stored in the plan dataset 629 can also be used to create rule sets.

An employer, pharmacy, PBM, or any plan administrator could be someone who uses the support interface 732 to set up rules for their associated cards (e.g., UPCs). The processing rules are pushed (e.g., scheduled, real-time, autonomously) as rule sets to the rules modeler 612. For example, a plan administrator can log into the support interface 732 (e.g., with an account previously registered) where they can create a rule for their members to always receive the lowest price drug. In another example, a pharmacy can log into the support interface 732 and create a rule to only use certain plans and not others. In yet another example, a discount card program can log into the support interface 732 and creates rules to use more profitable plans based on the drug or pharmacy.

In various implementations, created rules can be customized selection criteria specific to UPCs, providers, pharmacies, customers, etc. For example, a price rule could be for BIN 610679, select the plan with the lowest price for the customer. In another example, a pharmacy rule could be that if pharmacy is equal to provider A, do not use plan X. In yet another example, a drug rule could be that if drug is equal to Crestor use plan Y. In yet another, a customer assistant rule could be that if customer age is greater than sixty-four years of age and the claim state is equal to Texas and customer income is less than $45,000 then us plan Z. In yet another example, a generic effective Rate balancer rule could be that if the drug is equal to generic, and Plan A GER is greater than the wholesale price use Plan B. In some implementations, the created rules can be utilized by rules modeler 612 to create individual rule sets unique to UPCs, providers, pharmacies, customers, etc.

Plan dataset 629 can store various data received from the card allocation system 614, discount plan interface 728, and insurance plan interface 730. In some implementations, the discount plan interface 728 can be configured to collect, generate, and receive discount plan data and drug pricing data. The discount plan data and drug pricing data can be stored in plan dataset 629. In various implementations, the insurance plan interface 730 can be configured to collect, generate, and receive insurance plan data and copay pricing data. The Insurance plan data and copay pricing data can also be stored in plan dataset 629.

In some implementations, the card allocation system 614 can be configured to provide a graphical user interface for a user (e.g., customers, insurers, prescribers, pharmacists, caregivers) to register and receive an issued UPC. For example, a customer device (e.g., mobile phone, desktop computer, laptop) can interact with a website (e.g., using an internet browser) that provides graphical user interface. The user can be requested to provide information such as personal information (e.g., names, addresses, phone numbers, age, income, ethnicity, insurance plan information, and so on), authentication information (e.g., username/password combinations, device authentication tokens, security question answers, unique customer identifiers, biometric data, geographic data, social media data, etc.), and financial information (e.g., token information, account numbers, account balances, etc.) relating to the user. In various implementations, the card allocation system 614 can generate a UPC for one or more users (e.g., individual and/or bulk) based on the provided information. The UPC can include various routing information to route prescription drug claims to the claim editor 670. For example, the routing information can include, but is not limited to, a BIN, PCN, Group ID, Cardholder ID, unique CVC. In some implementations, the generated UPC can be linked to an insurance plan associated with the user. The received information and generated UPCs can be stored in plan dataset 629 and/or cardholder dataset 622. In one example, an employer can upload employee's information to card allocation system 614 (e.g., via network 630) for generation of a UPC for at least one (e.g., each) employee. In another example, a prescriber can provide a request for a UPC to the card allocation system 614 (e.g., via network 630) after a customer's first office visit.

In various implementations, the cardholder dataset 622 can store, but not limited to, user profile information (e.g., names, age, income, etc.), the user's UPC, insurance information (e.g., BIN, PCN, Group, ID), rule preference(s) (e.g., pick the lowest patient paid amount), plan preferences (e.g., only use X, Y, Z plans), and communication and data access opt-ins, and so on. In some implementations, the plan dataset 629 can store, but not limited to, various PBM discount and insurance plan information, plan details (BIN, PCN, Group ID, Cardholder ID), and drug pricing for cash pay and insurance (e.g., if not getting to price by adjudicating the claim real-time).

Interface system 616 (shown in FIG. 6) can be configured to communicate via network 630, for example with claim editor 670, PBMs 640, pharmacies 650, and/or data sources 660. For illustration purposes, at least one (e.g., each) interface is shown to be implemented as separate systems. However, it should be understood that the interface system 616 could include at least one (e.g., each) interface in a single system. In general, at least one (e.g., each) interface (e.g., 720, 722, 724, 726, 728, 730, and 732) can be configured to provide an interface to a customer, user, or another system described herein, for a particular task and/or action. In some implementations, a user device (e.g., user 702, 704, 706 with reference to FIG. 7) can execute a web browser application, which is provided an interface on a viewport of the user device. The web browser application that provides the interface can operate by receiving input of a uniform resource locator (URL), such as a web address, from an input device (e.g., a pointing device, a keyboard, a touch screen, or another form of input device). In response, one or more processors of the user device executing the instructions from the web browser application can request data and/or provide data from another device connected to the network 630 referred to by the URL address (e.g., claim controller 610). The other device can then provide webpage data and/or other data to the user device, which causes the interface to be presented by the viewport of the user device 101. Accordingly, the browser window presents the interface to facilitate user interaction with the interfaces described herein.

Reporting interface 722 can be configured to allow users (e.g., customers, insurance companies, self-insured employer groups, prescribers, caregivers, etc.) to view data visualization tools (e.g., historic claim activity across multiple PBMs and plans). For example, a self-insured employer (e.g., user 702) can run a report to analyze how much they saved on prescription drug costs by having one or more employees utilize a UPC. In another example, a customer or insurance company could access the reporting interface 722 (e.g., via a user device such as processing circuit 900) to calculate remaining deductible amounts. In yet another example, a prescriber could access the reporting interface 722 to view a customer's claim history to monitor prescription adherence.

Engagement interface 720 can be configured to communicate (e.g., via network 630) with user 702 based on event triggers. Event triggers notification can include, but is not limited to, pricing information or changes, savings, refill notifications, adherence and abandonment information, deductible notifications, and prior authorization notices. For example, a message (e.g., text, email, application notification) can be sent to user 702 (e.g., customer), over network 630, notifying user 702 when they have successfully linked their insurance plan to their UPC. In some implementations, incentives, like gift cards, can be sent to user 702 to encourage certain behaviors (e.g., pick up a medication, see a doctor, etc.).

Also as shown, the reporting interface 722 and engagement interface 720 can query the user dataset 626 and activity dataset 628 for various data that is provided in the data visualization tools and/or notifications/event triggers. In some implementations, the user dataset 626 can store credentials (e.g., login information) for people authorized to access the reporting interface 722. In various implementations, the reporting interface 722 can be configured to access and/or query the activity dataset 628 for the claim routing logs/activity.

User delivery interface 724 and rule delivery interface 726 can be configured to providing data for make routing decisions to claim editor 670 (e.g., where transactions can happen in milliseconds). In particular, the user delivery interface 724 can push (or transmit) data such as, but not limited to, user's UPC, insurance information (e.g., BIN, PCN, Group, ID), rule preference(s) (e.g., pick the lowest patient paid amount), and plan preferences (e.g., only use X, Y, Z plans), whereas the rule delivery interface 726 can push (or transmit) data to the router rules dataset 684 such as, but not limited to, the plan (e.g., BIN, PCN, Group ID, and Cardholder ID) with the lowest price at a particular pharmacy for a particular drug. Furthermore, both the user delivery interface 724 and the rule deliver interface 726 can update the card dataset 682 and router rules dataset 684 when any changes were detected (e.g., entity provides updated information, customer modifies a preference, customer changes their job, PBM modifies formularies, etc.)

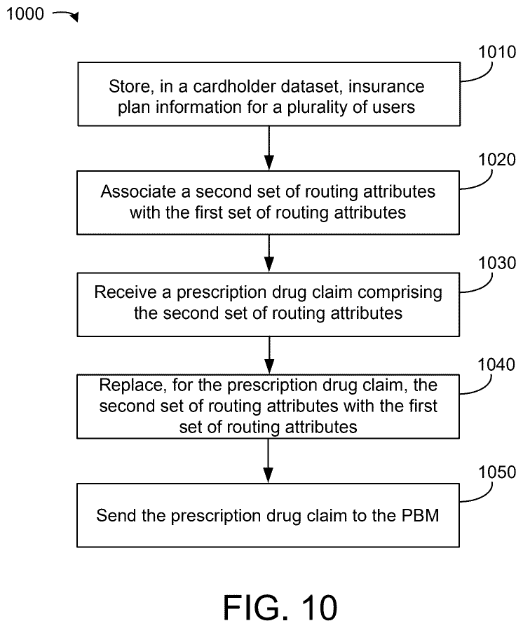

Referring now to FIG. 10, a flowchart for a method 1000 for routing prescription drug claims between a pharmacy and a plurality of pharmacy benefit managers (PBMs). Claim controller 610, claim editor 670, and associated environment 600 can be configured to perform method 1000.

In broad overview of method 1000, at block 1010, one or more processing circuits of a prescription drug claim routing system (e.g., claim editor 670 in FIG. 6, computer system 1200 in FIG. 12) can receive a prescription drug claim. At block 1020, the one or more processing circuits can obtain a set of rules for routing the prescription drug claim. At block 1030, the one or more processing circuits can select a PBM from the plurality of PBMs. At block 1040, the one or more processing circuits can modify the routing information of the prescription drug claim. At block 1050, the one or more processing circuits can send the prescription drug claim to the selected PBM. Additional, fewer, or different operations can be performed depending on the particular arrangement. In some arrangements, some or all operations of method 1000 can be performed by one or more processors executing on one or more computing devices, systems, or servers. In various arrangements, at least one (e.g., each) operation can be re-ordered, added, removed, or repeated.

Referring to method 1000 in more detail. At block 1010, the one or more processing circuits (e.g., claim editor 670 in FIG. 6) can receive a prescription drug claim from the pharmacy at a prescription drug claim routing system, the prescription drug claim including routing information that causes the pharmacy to send the prescription drug claim to the prescription drug claim routing system. The routing information can include attributes and fields associated with various entities and/or users (e.g., pharmacy identifying information, type of drug requested, insurance plan information, group identifiers, charge information, routing addresses, etc.) throughout the drug claim submission process.

At block 1020, the one or more processing circuits can obtain a set of rules for routing the prescription drug claim. In some implementations, the set of rules can be generated and provided by the claim controller 610. The set of rules can define a set of available prescription plan options for processing (or adjudicating) the prescription drug claim, at least one (e.g., each) of the available prescription plan options corresponding to a PBM of the plurality of PBMs.

At block 1030, the one or more processing circuits can select a PBM from the plurality of PBMs by applying the set of rules to the prescription drug claim. In various implementations, selecting the PBM from the plurality of PBMs includes evaluating the set of available prescription plan options according to the set of rules to select a prescription plan from the set of available prescription plan options. In some implementations, the selected PBM provides multiple different prescription plans. In such scenario, selecting the PBM includes selecting a prescription plan from the multiple different prescription plans provided by the selected PBM by applying the set of rules to the multiple different prescription plans. Furthermore, selecting the PBM from the plurality of PBMs can include using one or more attributes (or fields) of the prescription drug claim to identify an insurance plan applicable to the prescription drug claim and applying the set of rules to the insurance plan and one or more alternative prescription plans applicable to the prescription drug claim.

At block 1040, the one or more processing circuits can modify the routing information of the prescription drug claim to include updated routing information corresponding to the selected PBM. For example, the ID, GRP, BIN, and PCN can be modified based on the selected PBM.

At block 1050, the one or more processing circuits can send the prescription drug claim to the selected PBM for processing in accordance with the updated routing information. In various implementations, the claim editor 670 can send the prescription drug claim to a PBM of PBMs 640. In some implementations, the prescription drug claim can be sent to a plurality of PBMs for processing such that the claim editor 670 can analyze one or more PBMs response and apply the customized selection criteria. Additionally, the one or more processing circuits can receive a claim response from the selected PBM responsive to sending the prescription drug claim to the selected PBM for processing. The one or more processing circuits can modify the claim response from the selected PBM to include information identifying the selected PBM and send the modified claim response to the pharmacy.

In some implementations, the pharmacy 650 and the claim editor 670 can be implemented as separate systems or integrated within a single system (e.g., the pharmacy can be configured to incorporate some or all of the functions/capabilities of the claim editor 670). In the following implementation, the pharmacy 650 can generate a prescription drug claim, obtain a set of rules for routing the prescription drug claim, select a PBM from the plurality of PBMs by applying the set of rules to the prescription drug claim, modify the prescription drug claim to include routing information corresponding to the selected PBM, and send the prescription drug claim to the selected PBM for processing in accordance with the routing information.

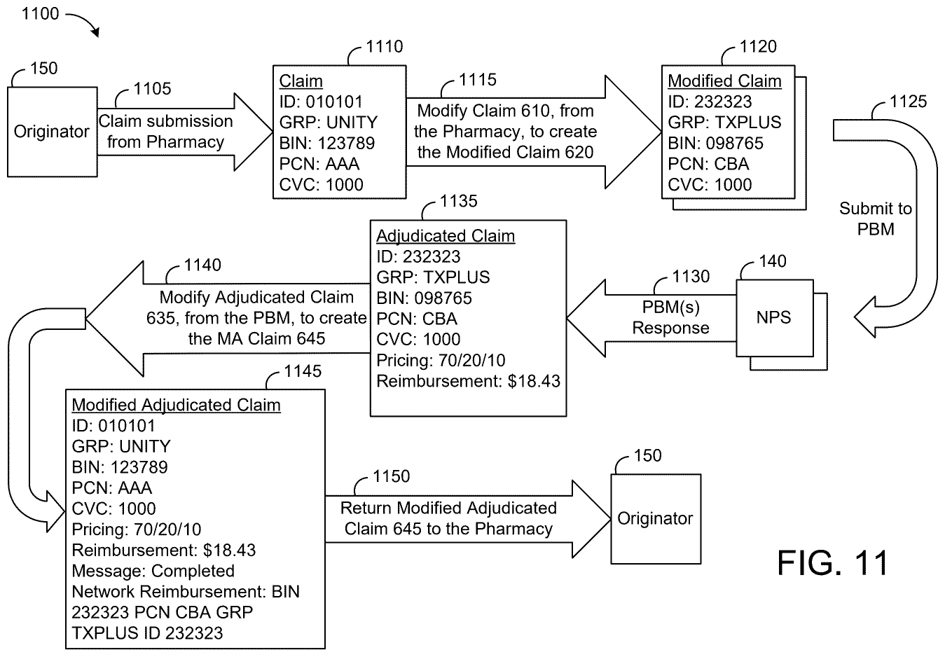

Referring now to FIG. 11, a block diagram depicting an example of a claim processing workflow 1100 in connection with the drug claim processing architecture of FIGS. 6-7 is shown, according to an illustrative implementation. In some implementations, a pharmacy (e.g., 1150) can submit 1105 a claim 1110 to claim routing system 105. The claim editor 1170 can receive the drug claim 1110 and modify (modification 1115) the claim 1110 based on the rule set and the selected one or more PBMs 140 to generate a modified claim 1120. In some implementations, the modification 1115 can include updating the routing information to send the modified claim 1120 to the select PBM(s) 140. As shown and in some implementations, the modified claim 1120 can be modified a plurality of times (e.g., unique to a PBM) when the claim editor 1170 determines the prescription drug claim should be sent to more than one PBM. The PBM(s) 140 can provide a response 1130 with an adjudicated claim 1135 to claim routing system 105. The claim editor 1170 can receive the adjudicated claim 1135 and modify (modification 1140) the adjudicated claim 1135 based on the rule set and the pharmacy 1150 to generate a modified adjudicated claim 1145. In some implementations, the modification 1140 can include updating the routing information to send the modified adjudicated claim 1145 to the pharmacy 1150. As shown, the modified adjudicated drug claim 1145 can be sent 1150 back to pharmacy 1150 with reimbursement information and/or a message (e.g., dispensing info, copay, out-of-pocket cost for customer, failure to process by PBM, etc.).

FIG. 12 illustrates a depiction of a computer system 1200 that can be used, for example, to implement a claim controller 610, controller database 120, PBMs 640, a pharmacies 650, a claim editor 670, a router database 680, and/or various other example systems described in the present disclosure. The computing system 1200 includes a bus 1205 or other communication component for communicating information and a processor 1210 coupled to the bus 1205 for processing information. The computing system 1200 also includes main memory 1215, such as a random-access memory (RAM) or other dynamic storage device, coupled to the bus 1205 for storing information, and instructions to be executed by the processor 1210. Main memory 1215 can also be used for storing position information, temporary variables, or other intermediate information during execution of instructions by the processor 1210. The computing system 1200 can further include a read only memory (ROM) 1220 or other static storage device coupled to the bus 1205 for storing static information and instructions for the processor 1210. A storage device 1225, such as a solid-state device, magnetic disk or optical disk, is coupled to the bus 1205 for persistently storing information and instructions.

Computing system 1200 can be coupled via the bus 1205 to a display 1235, such as a liquid crystal display, or active matrix display, for displaying information to a user. An input device 1230, such as a keyboard including alphanumeric and other keys, can be coupled to the bus 1205 for communicating information, and command selections to the processor 1210. In another arrangement, the input device 1230 has a touch screen display 1235. The input device 1230 can include any type of biometric sensor, a cursor control, such as a mouse, a trackball, or cursor direction keys, for communicating direction information and command selections to the processor 1210 and for controlling cursor movement on the display 1235.

In some arrangements, the computing system 1200 can include a communications adapter 1240, such as a networking adapter. Communications adapter 1240 can be coupled to bus 1205 and can be configured to facilitate communications with a computing or communications network 130 and/or other computing systems. In various illustrative arrangements, any type of networking configuration can be achieved using communications adapter 1240, such as wired (e.g., via Ethernet), wireless (e.g., via WiFi, Bluetooth, and so on), satellite (e.g., via GPS) pre-configured, ad-hoc, LAN, WAN, and so on.

According to various arrangements, the processes that effectuate illustrative arrangements that are described herein can be achieved by the computing system 1200 in response to the processor 1210 executing an arrangement of instructions contained in main memory 1215. Such instructions can be read into main memory 1215 from another computer-readable medium, such as the storage device 1225. Execution of the arrangement of instructions contained in main memory 1215 causes the computing system 1200 to perform the illustrative processes described herein. One or more processors in a multi-processing arrangement can also be employed to execute the instructions contained in main memory 1215. In alternative arrangements, hard-wired circuitry can be used in place of or in combination with software instructions to implement illustrative arrangements. Thus, arrangements are not limited to any specific combination of hardware circuitry and software.

That is, although an example processing system has been described in FIG. 12, arrangements of the subject matter and the functional operations described in this specification can be carried out using other types of digital electronic circuitry, or in computer software embodied on a tangible medium, firmware, or hardware, including the structures disclosed in this specification and their structural equivalents, or in combinations of one or more of them. Arrangements of the subject matter described in this specification can be implemented as one or more computer programs, e.g., one or more subsystems of computer program instructions, encoded on one or more computer storage medium for execution by, or to control the operation of, data processing apparatus. Alternatively, or in addition, the program instructions can be encoded on an artificially generated propagated signal, e.g., a machine generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices). Accordingly, the computer storage medium is both tangible and non-transitory.

The operations described in this specification can be implemented as operations performed by a data processing apparatus on data stored on one or more computer-readable storage devices or received from other sources.

The terms “data processing system” or “processor” encompass all kinds of apparatus, devices, and machines for processing data, including by way of example, a programmable processor, a computer, a system on a chip, or multiple ones, or combinations of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more subsystems, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example, semiconductor memory devices, e.g., EPROM, EEPROM, and flash memory devices, magnetic disks, e.g., internal hard disks or removable disks, magneto-optical disks, and CD-ROM and DVD-ROM disks. The processor and the memory can be supplemented by, or incorporated in, special purpose logic circuitry.

To provide for interaction with a user, arrangements of the subject matter described in this specification can be carried out using a computer having a display device, e.g., a quantum dot display (QLED), organic light-emitting diode (OLED), or liquid crystal display (LCD) monitor, for displaying information to the user and a keyboard and a pointing device, e.g., a mouse or a trackball, by which the user can provide input to the computer. Other kinds of devices can be used to provide for interaction with a user as well, for example, feedback provided to the user can be any form of sensory feedback, e.g., visual feedback, auditory feedback, or tactile feedback, and input from the user can be received in any form, including acoustic, speech, tactile input, or other biometric information. In addition, a computer can interact with a user by sending documents to and receiving documents from a device that is used by the user, for example, by sending web pages to a web browser on a user's customer device in response to requests received from the web browser.

Arrangements of the subject matter described in this specification can be carried out using a computing system that includes a back-end component, e.g., as a data server, or that includes a middleware component, e.g., an application server, or that includes a front-end component, e.g., a client (or customer) computer having a graphical user interface or a web browser through which a user can interact with an arrangement of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from at least one (e.g., each) other and typically interact through a communication network 630. The relationship of client and server arises by virtue of computer programs running on the respective computers and having a client-server relationship to at least one (e.g., each) other. In some arrangements, a server transmits data (e.g., an HTML page) to a client device (e.g., for purposes of displaying data to and receiving user input from a user interacting with the client device). Data generated at the client device (e.g., a result of the user interaction) can be received from the client device (or customer device) at the server.

While this specification contains many specific implementation details and/or arrangement details, these should not be construed as limitations on the scope of any inventions or of what can be claimed, but rather as descriptions of features specific to particular implementations and/or arrangements of the systems and methods described herein. Certain features that are described in this specification in the context of separate implementations and/or arrangements can also be implemented and/or arranged in combination in a single implementation and/or arrangement. Conversely, various features that are described in the context of a single implementation and/or arrangement can also be implemented and arranged in multiple implementations and/or arrangements separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Additionally, features described with respect to particular headings can be utilized with respect to and/or in combination with illustrative arrangement described under other headings, headings, where provided, are included solely for the purpose of readability and should not be construed as limiting any features provided with respect to such headings.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing can be advantageous. Moreover, the separation of various system components in the implementations and/or arrangements described above should not be understood as requiring such separation in all implementations and/or arrangements, and it should be understood that the described program components and systems can generally be integrated together in a single software product or packaged into multiple software products.

Having now described some illustrative implementations, implementations, illustrative arrangements, and arrangements it is apparent that the foregoing is illustrative and not limiting, having been presented by way of example. In particular, although many of the examples presented herein involve specific combinations of method acts or system elements, those acts, and those elements can be combined in other ways to accomplish the same objectives. Acts, elements, and features discussed only in connection with one implementation and/or arrangement are not intended to be excluded from a similar role in other implementations or arrangements.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations and/or arrangements consisting of the items listed thereafter exclusively. In one arrangement, the systems and methods described herein consist of one, at least one (e.g., each) combination of more than one, or all of the described elements, acts, or components.

Any references to implementations, arrangements, or elements or acts of the systems and methods herein referred to in the singular can also embrace implementations and/or arrangements including a plurality of these elements, and any references in plural to any implementation, arrangement, or element or act herein can also embrace implementations and/or arrangements including only a single element. References in the singular or plural form are not intended to limit the presently disclosed systems or methods, their components, acts, or elements to single or plural configurations. References to any act or element being based on any information, act or element can include implementations and/or arrangements where the act or element is based at least in part on any information, act, or element.

Any implementation disclosed herein can be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation can be included in at least one implementation. Such terms as used herein are not necessarily all referring to the same implementation. Any implementation can be combined with any other implementation, inclusively or exclusively, in any manner consistent with the aspects and implementations disclosed herein.

Any arrangement disclosed herein can be combined with any other arrangement, and references to “an arrangement,” “some arrangements,” “an alternate arrangement,” “various arrangements,” “one arrangement” or the like are not necessarily mutually exclusive and are intended to indicate that a particular feature, structure, or characteristic described in connection with the arrangement can be included in at least one arrangement. Such terms as used herein are not necessarily all referring to the same arrangement. Any arrangement can be combined with any other arrangement, inclusively or exclusively, in any manner consistent with the aspects and arrangements disclosed herein.

References to “or” can be construed as inclusive so that any terms described using “or” can indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein can be embodied in other specific forms without departing from the characteristics thereof. Although the examples provided herein relate to controlling the display of content of information resources, the systems and methods described herein can include applied to other environments. The foregoing implementations and/or arrangements are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. A computer network routing system for routing packages between a computer network originator system and a plurality of computer network processing systems, the computer network routing system comprising:
one or more processing circuits comprising one or more processors and memory storing instructions that, when executed by the one or more processors, cause the one or more processors to perform operations comprising:
storing, in a network user dataset, network plan information for a plurality of users, the network plan information for at least one user of the plurality of users comprising a first set of routing attributes;
associating, in the network user dataset, a second set of routing attributes with the first set of routing attributes for the at least one user, the second set of routing attributes configured for use by the computer network originator system when submitting the packages;
receiving, from the computer network originator system, a package comprising the second set of routing attributes associated with the at least one user;
replacing, for the package, the second set of routing attributes with the first set of routing attributes associated with the at least one user to generate updated routing information corresponding to a computer network processing system of the plurality of computer network processing systems; and
sending the package comprising the updated routing information to the computer network processing system for processing.

2. The computer network routing system of claim 1, wherein the first set of routing attributes comprises at least one of an original bank identification number (BIN), an original processor control number (PCN), an original group identifier, and an original member identifier associated with a network plan of the at least one user.

3. The computer network routing system of claim 2, wherein the second set of routing attributes comprises at least one of a new BIN, a new PCN, a new group identifier, and a new member identifier associated with a prescription card generated for the at least one user.

4. The computer network routing system of claim 3, wherein:
the package comprising the updated routing information generated by a BIN over BIN translation replacing the second set of routing attributes with the first set of routing attributes comprises replacing the new BIN, the new PCN, the new group identifier, and the new member identifier with the original BIN, the original PCN, the original group identifier, and the original member identifier of original network plan information corresponding to the at least one user, the updated routing information corresponding to the computer network processing system of the plurality of computer network processing system.

5. The computer network routing system of claim 4, wherein the operations further comprise:
responsive to receiving a claim response from the computer network processing system for the package comprising the updated routing information, generating, without submitting any additional package to any computer network processing system, a notification for the at least one user, the notification comprising a final cost of a prescribed drug under an network plan associated with the original bank identification number, the original processor control number, the original group identifier, and the original member identifier, and pricing information for at least one lower-cost alternative.

6. The computer network routing system of claim 1, wherein:
receiving the package from the computer network originator system comprises receiving the package at a claim editor comprising a claim pre-editor; and
replacing the second set of routing attributes with the first set of routing attributes is performed by the claim pre-editor prior to sending the package to the computer network processing system.

7. The computer network routing system of claim 1, wherein the operations further comprise:
receiving, from the computer network processing system, a claim response responsive to the package comprising the updated routing information;
modifying the claim response to comprise information identifying the computer network processing system; and
sending the modified claim response to the computer network originator system.

8. The computer network routing system of claim 7, wherein:
modifying the claim response to comprise information identifying the computer network processing system comprises updating at least one response network segment field of the claim response with a network reimbursement identifier associated with the computer network processing system; and
modifying the claim response to comprise information identifying the computer network processing system comprises updating at least one response message segment field of the claim response with a message indicating a computer network processing system that adjudicated the package.

9. The computer network routing system of claim 7, wherein the operations further comprise:
logging, in a logging dataset, at least one of the package received from the computer network originator system, the package comprising the updated routing information sent to the computer network processing system, and the claim response received from the computer network processing system;
generating, for the at least one user associated with the package, a notification comprising pricing information derived from the claim response received from the computer network processing system; and
sending the notification to a user device associated with the at least one user via at least one of a text message, an email message, and a mobile application message.

10. The computer network routing system of claim 9, wherein the pricing information comprises at least one of:
a patient pay amount for a prescribed drug under a network plan associated with the first set of routing attributes;
pricing for one or more alternative prescription plans associated with one or more computer network processing systems; and
pricing for one or more discount plans applicable to the prescribed drug;
wherein the notification further comprises information describing at least one copay assistance program applicable to the prescribed drug based on at least one attribute of the at least one user stored in the network user dataset.

11. The computer network routing system of claim 10, wherein the notification further comprises information describing a lower-cost alternative to the prescribed drug, the lower-cost alternative comprising at least one of:
an on-formulary alternative drug;
a generic version of the prescribed drug; and
a different computer network originator system at which a lower price is available.

12. The computer network routing system of claim 11, wherein the operations further comprise:
obtaining, from one or more data sources, plan data comprising at least one of discount plan data and network plan data; and
storing the plan data in a plan dataset for use in determining at least one of the pricing information and the lower-cost alternative provided to the at least one user.

13. The computer network routing system of claim 1, wherein:
storing the network plan information for the plurality of users comprises storing, in the network user dataset, the first set of routing attributes comprising original routing attributes; and
associating the second set of routing attributes with the first set of routing attributes for the at least one user comprises storing, in the network user dataset, a mapping between the second set of routing attributes comprising new routing attributes and the first set of routing attributes comprising the original routing attributes for the at least one user.

14. The computer network routing system of claim 13, wherein:
receiving, from the computer network originator system, the package comprising the second set of routing attributes associated with the at least one user comprises intercepting, at the one or more processing circuits, the package from the computer network originator system prior to sending the package to the computer network processing system; and
replacing, for the package, the second set of routing attributes with the first set of routing attributes associated with the at least one user comprises generating the updated routing information based on the mapping stored in the network user dataset.

15. The computer network routing system of claim 1, wherein:
sending the package comprising the updated routing information to the computer network processing system for processing comprises routing, in real time, the package comprising the updated routing information to the computer network processing system of the plurality of computer network processing systems based on the first set of routing attributes associated with the at least one user; and
data transmissions associated with the package and the network plan information are encrypted in transit and data stored in the network user dataset is encrypted at rest.

16. The computer network routing system of claim 1, wherein:
storing the network plan information comprises receiving the first set of routing attributes from at least one of an employer system and a user device; and
associating the second set of routing attributes with the first set of routing attributes comprises generating a prescription card for the at least one user comprising the second set of routing attributes.

17. A method, comprising:
storing, by one or more processing circuits in a network user dataset, network plan information for a plurality of users, the network plan information for at least one user of the plurality of users comprising a first set of routing attributes;
associating, by the one or more processing circuits in the network user dataset, a second set of routing attributes with the first set of routing attributes for the at least one user, the second set of routing attributes configured for use by a computer network originator system when submitting packages;
receiving, by the one or more processing circuits from the computer network originator system, a package comprising the second set of routing attributes associated with the at least one user;
replacing, by the one or more processing circuits for the package, the second set of routing attributes with the first set of routing attributes associated with the at least one user to generate updated routing information corresponding to a computer network processing system of a plurality of computer network processing systems; and
sending, by the one or more processing circuits, the package comprising the updated routing information to the computer network processing system for processing.

18. The method of claim 17, wherein the first set of routing attributes comprises at least one of an original bank identification number (BIN), an original processor control number (PCN), an original group identifier, and an original member identifier associated with an network plan of the at least one user, and wherein the second set of routing attributes comprises at least one of a new BIN, a new PCN, a new group identifier, and a new member identifier associated with a prescription card generated for the at least one user.

19. The method of claim 18, wherein:
the package comprising the updated routing information generated by a BIN over BIN translation replacing the second set of routing attributes with the first set of routing attributes comprises replacing the new BIN, the new PCN, the new group identifier, and the new member identifier with the original BIN, the original PCN, the original group identifier, and the original member identifier of original network plan information corresponding to the at least one user, the updated routing information corresponding to the computer network processing system of the plurality of computer network processing systems.

20. A computer network routing system for routing packages between a computer network originator system and a plurality of computer network processing systems, the computer network routing system comprising:
one or more processing circuits comprising one or more processors and memory storing instructions that, when executed by the one or more processors, cause the one or more processors to perform operations comprising:
storing, in a network user dataset, network plan information in a standardized routing format for a plurality of users, the network plan information for at least one user of the plurality of users comprising a first set of routing attributes in the standardized routing format;
providing, over a computer network, remote access to the computer network originator system so that the computer network originator system can submit the packages in real time using routing information in a non-standardized format dependent on a hardware and/or software platform of the computer network originator system, the routing information in the non-standardized format comprising a second set of routing attributes associated with the at least one user;
converting, by the one or more processing circuits, the second set of routing attributes in the non-standardized format into the first set of routing attributes in the standardized routing format by replacing, for each received package, the second set of routing attributes with the first set of routing attributes associated with the at least one user to generate updated routing information corresponding to a computer network processing system of the plurality of computer network processing systems;
storing, in the network user dataset, the updated routing information for the at least one user in the standardized routing format; and
sending, over the computer network and in real time, a package comprising the updated routing information in the standardized routing format to the computer network processing system for processing.
